(19) United States
(12) Patent Application Publication
(10) Pub. No.: US 2004/0138230 A1
(43) Pub. Date: Jul. 15, 2004

(54) Heterocyclic substituted piperazines for the treatment of schizophrenia

(76) Inventors: Tonja Lynn Andreana, Somerville, MA (US); Stephen Sung Yong Cho, Saline, MI (US); James M. Graham, Ann Arbor, MI (US); Tracy Fay Gregory, Parma, MI (US); Harry Ralph Howard JR., Bristol, CT (US); Brian Edward Kornberg, Ann Arbor, MI (US); Sham Shridhar Nikam, Ann Arbor, MI (US); Derek Andrew Pflum, Northville, MI (US)

Correspondence Address: WARNER-LAMBERT COMPANY, 2800 PLYMOUTH RD, ANN ARBOR, MI, 48105, US

(21) Appl. No.: 10/660,908

(22) Filed: Sep. 12, 2003

Related U.S. Application Data
(60) Provisional application No. 60/411,475, filed on Sep. 17, 2002.
(60) Provisional application No. 60/416,355, filed on Oct. 4, 2002.

(51) Int. Cl. A61K 31/496; C 07D 4 3/14

(52) U.S. Cl. 514/253.07; 544/363; 514/314; 546/157

(57) ABSTRACT

This invention relates to compounds of the formula 1

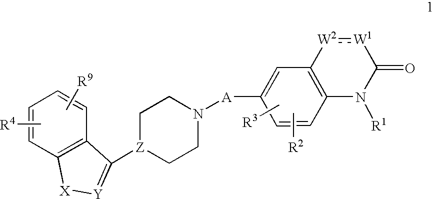

wherein X, Y, Z, A, R¹, R², R³, R⁴, R⁹, W¹ and W² are defined as in the specification, pharmaceutical compositions containing them and their use in the treatment of central nervous system and other disorders.

### BACKGROUND OF THE INVENTION

This invention relates to heterocyclic substituted piperazines, pharmaceutical compositions containing them and their use for the treatment of schizophrenia and other central nervous system (CNS).

The heterocyclic substituted piperazine derivatives of this invention exhibit activity as antagonists of dopamine D2 receptors and of serotonin 2A (5HT2A) receptors.

Other heterocyclic piperazine derivatives that are useful for the treatment of schizophrenia are referred to in U.S. Pat. No. 5,350,747, which issued on Sep. 27, 1994, and in U.S. Pat. No. 6,127,357, which issued on Oct. 3, 2000. These patents are incorporated herein by reference in their entireties.

Other piperazine and piperidine derivatives that have been stated to be useful as antipsychotic agents are those referred to in PCT patent publication WO 93/04684, which published on Mar. 18, 1993, and European patent application EP 402644A, which was published on Dec. 19, 1990. These patent applications are incorporated herein by reference in their entireties.

### SUMMARY OF THE INVENTION

The present invention relates to compounds of the formula 1

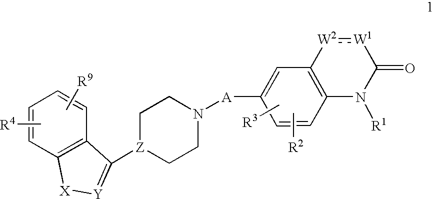

wherein X is sulfur, oxygen, SO, SO₂, CH₂ or NR¹⁰;

Y is nitrogen or CH;

Z is nitrogen or CH;

A is —(CH₂)ₘCH₂—, —(CH₂)ₘO—, —(CH₂)ₘNR¹¹—, or —(CH₂)ₘC(R¹²R¹³)—, wherein R¹² and R¹³ are independently selected from (C₁-C₄) alkyl optionally substituted with from one to three fluorine atoms, (C₁-C₄) alkoxy optionally substituted with from one to three fluorine atoms, hydroxy, and aminoalkyl;

or R¹² and R¹³, together with the carbon to which they are attached, form a carbonyl group;

m is an integer from one to four;

R⁴ and R⁹ are independently selected from hydrogen, (C₁-C₄) alkyl optionally substituted with from one to three fluorine atoms, (C₁-C₄) alkoxy optionally substituted with from one to three fluorine atoms, halogen, nitro, cyano, amino, (C₁-C₄) alkylamino and di-(C₁-C₄) alkylamino;

or, when X is NR¹⁰, one of R⁴ and R⁹ can form, together with the carbon to which it is attached, and together with R¹⁰ and the nitrogen to which it is attached, a heterocyclic ring containing from 4 to 7 ring members of which from 1 to 3 ring members are heteroatoms selected from nitrogen, oxygen and sulfur, and of which the remaining ring members are carbon, with the proviso that when R¹¹ forms a ring with one of R⁴ and R⁹, the other of R⁴ and R⁹ is absent;

R¹⁰ and R¹¹ are independently selected from hydrogen, (C₁-C₄) alkyl optionally substituted with from one to three fluorine atoms and (C₁-C₄) alkoxy optionally substituted with from one to three fluorine atoms.

R¹ is hydrogen, (C₁-C₄) alkyl optionally substituted with from one to three fluorine atoms, aryl, —C(O)R¹⁴ wherein R¹⁴ is aryl, (C₁-C₄) alkyl, aryl-(C₁-C₄) alkyl-, or heteroaryl-(C₁-C₄)alkyl-, wherein the alkyl moieties of the aryl-(C₁-C₄) alkyl- groups and the heteroaryl-(C₁-C₄) alkyl- groups can be optionally substituted with from one to three fluoro atoms, and wherein the aryl and heteroaryl moieties of these groups can optionally be substituted with one or more substituents, preferably with from zero to two substituents, independently selected from halo, nitro, amino, cyano, (C₁-C₆) alkyl optionally substituted with from one to three fluorine atoms and (C₁-C₆) alkoxy optionally substituted with from one to three fluorine atoms;

R² and R³ are independently selected from hydrogen, halo, (C₁-C₄) alkyl, (C₁-C₄) alkoxy, aryl, aryl-(C₁-C₄) alkyl-, heteroaryl and heteroaryl-(C₁-C₄) alkyl-, wherein the alkyl moieties of the (C₁-C₄) alkyl and (C₁-C₄) alkoxy groups can be optionally substituted with from one to three fluoro atoms and can also be independently optionally substituted with an amino or hydroxy substituent, and wherein alkyl moieties of the aryl-(C₁-C₄) alkyl- and heteroaryl-(C₁-C₄) alkyl groups can be optionally substituted with from one to three fluoro atoms, and wherein the aryl and heteroaryl moieties of these groups can optionally be substituted with one or more substituents, preferably from zero to two substituents, independently selected from halo, nitro, amino, cyano, (C₁-C₆) alkyl optionally substituted with from one to three fluorine atoms and (C₁-C₆) alkoxy optionally substituted with from one to three fluorine atoms;

or one of R² and R³ can form, together with the carbon to which it is attached, and together with the quinolinone ring carbon of W¹, a saturated or unsaturated heterocyclic ring containing from 4 to 7 ring members of which from 1 to 3 ring members can be heteroatoms selected from nitrogen, oxygen and sulfur, and of which the remaining ring members are carbon, with the proviso that when W¹ forms a ring with one of R² and R³, the other of R² and R³ is absent;

W¹ is CR⁵R⁶ and W² is CR⁷R⁸, and the broken line extending from W¹ to W² represents an optional double bond, with the proviso that when the there is a double bond between W¹ and W², R⁵ and R⁷ are absent;

R⁵, R⁶, R⁷, and R⁸ selected, independently, from hydrogen, halogen, nitro, cyano, amino, (C₁-C₄) alkylamino, di-(C₁-C₄) alkylamino, (C₁-C₄) alkyl optionally substituted with from one to three fluorine atoms, and (C₁-C₄) alkoxy optionally substituted with from one to three fluorine atoms;

or any two of R⁵, R⁶, R⁷, and R⁸ that are attached to carbon atoms, taken together with the carbon or carbons to which they are attached, form a (C₃-C₇) saturated or unsaturated carbocyclic ring or a (C₅-C₇) saturated or unsaturated heterocyclic ring wherein one or two of the ring members are selected from nitrogen, oxygen and sulfur, with the proviso that the quinolinone ring carbon of W¹ can not form a ring with two of R⁵, R⁶, R⁷, and R⁸ and also form a ring with R² or R³;

and the pharmaceutically acceptable salts of such compounds.

Another more specific embodiment of this invention relates to compounds of the formula 1, and their pharmaceutically acceptable salts, wherein A is —(CH₂)ₘO—.

Another more specific embodiment of this invention relates to compounds of the formula 1, and their pharmaceutically acceptable salts, wherein A is —(CH₂)ₘNR¹¹—.

Another more specific embodiment of this invention relates to compounds of the formula 1, and their pharmaceutically acceptable salts, wherein X is sulfur.

Another more specific embodiment of this invention relates to compounds of the formula 1, and their pharmaceutically acceptable salts, wherein X is SO or SO₂.

Another more specific embodiment of this invention relates to compounds of the formula 1, and their pharmaceutically acceptable salts, wherein X is CH₂ or NR.

Examples of preferred embodiments of this invention are the following compounds and their pharmaceutically acceptable salts:

6-[2-(4-benzo[d]isoxazol-3-yl-piperazin-1-yl)-ethyl]-4-methyl-3,4-dihydro-1H-quinolin-2-one;

6-[2-(4-benzo[d]isoxazol-3-yl-piperazin-1-yl)-ethyl]-4S-methyl-3,4-dihydro-1H-quinolin-2-one;

6-[2-(4-benzo[d]isoxazol-3-yl-piperazin-1-yl)-ethyl]-4R-methyl-3,4-dihydro-1H-quinolin-2-one;

6-[2-(4-benzo[d]isoxazol-3-yl-piperazin-1-yl)-ethyl]-1,4-dimethyl-3,4-dihydro-1H-quinolin-2-one;

6-[2-(4-benzo[d]isoxazol-3-yl-piperazin-1-yl)-ethyl]-4,4-dimethyl-3,4-dihydro-1H-quinolin-2-one;

6-[2-(4-benzo[d]isoxazol-3-yl-piperazin-1-yl)-ethyl]-1,4,4-trimethyl-3,4-dihydro-1H-quinolin-2-one;

6-[2-(4-benzo[d]isoxazol-3-yl-piperazin-1-yl)-ethyl]-3-methyl-3,4-dihydro-1H-quinolin-2-one;

6-[2-(4-benzo[d]isoxazol-3-yl-piperazin-1-yl)-ethyl]-3,3-dimethyl-3,4-dihydro-1H-quinolin-2-one;

6-[2-(4-benzo[d]isoxazol-3-yl-piperazin-1-yl)-ethyl]-3,4-dimethyl-1H-quinolin-2-one;

6-[2-(4-benzo[d]isoxazol-3-yl-piperazin-1-yl)-ethyl]-3,4-dimethyl-3,4-dihydro-1H-quinolin-2-one;

6-[2-(4-benzo[d]isoxazol-3-yl-piperazin-1-yl)-ethyl]-1,3,3,4,4-pentamethyl-3,4-dihydro-1H-quinolin-2-one;

6-[2-(4-benzo[d]isoxazol-3-yl-piperazin-1-yl)-ethyl]-3,3,4-trimethyl-3,4-dihydro-1H-quinolin-2-one;

6-{2-[4-(1H-indazol-3-yl)-piperazin-1-yl]-ethyl}-4-methyl-3,4-dihydro-1H-quinolin-2-one;

6-{2-[4-(1H-indazol-3-yl)-piperazin-1-yl]-ethyl}-4,4-dimethyl-3,4-dihydro-1H-quinolin-2-one;

6-{2-[4-(1H-indazol-3-yl)-piperazin-1-yl]-ethyl}-3-methyl-3,4-dihydro-1H-quinolin-2-one;

6-{2-[4-(1H-indazol-3-yl)-piperazin-1-yl]-ethyl}-3,3-dimethyl-3,4-dihydro-1H-quinolin-2-one;

6-{2-[4-(1H-indazol-3-yl)-piperazin-1-yl]-ethyl}-3,4-dimethyl-3,4-dihydro-1H-quinolin-2-one;

6-[2-(4-benzo[d]isothiazol-3-yl-piperazin-1-yl)-ethyl]-1,3,3,4,4-pentamethyl-3,4-dihydro-1H-quinolin-2-one;

6-[2-(4-benzo[d]isothiazol-3-yl-piperazin-1-yl)-ethyl]-3,3,4-trimethyl-3,4-dihydro-1H-quinolin-2-one;

6-[2-(4-Benzo[d]isothiazol-3-yl-piperazin-1-yl)-ethyl]-4,4,8-trimethyl-3,4-dihydro-1H-quinolin-2-one, mesylate salt;

6-[2-(4-Benzo[d]isothiazol-3-yl-piperazin-1-yl)-ethyl]-7-chloro-4,4,8-trimethyl-3,4-dihydro-1H-quinolin-2-one methanesulfonate;

6-[2-(4-Benzo[d]isothiazol-3-yl-piperazin-1-yl)-ethyl]-7-fluoro-4,4,8-trimethyl-3,4-dihydro-1H-quinolin-2-one hydrochloride;

6-[2-(4-benzo[d]isothiazol-3-yl-piperazin-1-yl)-ethyl]-4,4,8-trimethyl-3,4-dihydro-1H-quinolin-2-one;

6-{3-[4-(1H-indazol-3-yl)-piperazin-1-yl]-propyl}-4,4-dimethyl-3,4-dihydro-1H-quinolin-2-one;

7-chloro-6-[3-(4-1,2-benzisothiazol-3-yl-piperazin-1-yl)-propyl]-4,4-dimethyl-3,4-dihydro-1H-quinolin-2-one;

7-chloro-6-[3-(4-1,2-benzisoxazol-3-yl-piperazin-1-yl)-propyl]-4,4-dimethyl-3,4-dihydro-1H-quinolin-2-one;

6-[3-(4-1,2-benzisothiazol-3-yl-piperazin-1-yl)-propyl]-4-methyl-3,4-dihydro-1H-quinolin-2-one;

6-[3-(4-1,2-benzisoxazol-3-yl-piperazin-1-yl)-propyl]-4-methyl-3,4-dihydro-1H-quinolin-2 one;

6-{3-[4-(1H-indazol-3-yl)-piperazin-1-yl]-propyl}-4-methyl-3,4-dihydro-1H-quinolin-2-one;

6-[3-(4-1,2-benzisothiazol-3-yl-piperazin-1-yl)-propyl]-3,3-dimethyl-3,4-dihydro-1H-quinolin-2-one;

6-[3-(4-1,2-benzisoxazol-3-yl-piperazin-1-yl)-propyl]-3,3-dimethyl-3,4-dihydro-1H-quinolin-2 one;

6-{3-[4-(1H-indazol-3-yl)-piperazin-1-yl]-propyl}-3,3-di methyl-3,4-dihydro-1H-quinolin-2-one;

6-[3-(4-1,2-benzisothiazol-3-yl-piperazin-1-yl)-propyl]-3-methyl-3,4-dihydro-1H-quinolin-2-one;

6-[3-(4-1,2-benzisoxazol-3-yl-piperazin-1-yl)-propyl]-3-methyl-3,4-dihydro-1H-quinolin-2 one; and

6-{3-[4-(1H-indazol-3-yl)-piperazin-1-yl]-propyl}-3-methyl-3,4-dihydro-1H-quinolin-2-one.

The above listed compounds are hereinafter referred to, collectively, as “the Group A compounds”.

The term “alkyl”, as used herein, unless otherwise indicated, includes saturated monovalent hydrocarbon radicals having straight, branched or cyclic moieties or combinations thereof. Examples of “alkyl” groups include, but are not limited to, methyl, ethyl, propyl, isopropyl, butyl, iso- sec- and tert-butyl, pentyl, hexyl, heptyl, 3-ethylbutyl, cyclopropyl, cyclobutyl, cyclopentyl, cyclohexyl, cycloheptyl, norbornyl, and the like.

The term “aryl”, as used herein, unless otherwise indicated, includes an aromatic ring system with no ring heteroatoms (e.g., phenyl or naphthyl).

The term “alkoxy”, as used herein, unless otherwise indicated, means “alkyl-O—”, wherein “alkyl” is as defined above. Examples of “alkoxy” groups include, but are not limited to, methoxy, ethoxy, propoxy, butoxy and pentoxy.

The term “alkenyl”, as used herein, unless otherwise indicated, includes unsaturated hydrocarbon radicals having one or more double bonds connecting two carbon atoms, wherein said hydrocarbon radical may have straight, branched or cyclic moieties or combinations thereof. Examples of “alkenyl” groups include, but are not limited to, ethenyl, propenyl, butenyl, pentenyl.

The term “heteroaryl”, as used herein, unless otherwise indicated, includes monocyclic aromatic heterocycles containing five or six ring members, of which from 1 to 4 can be heteroatoms selected, independently, from N, S and O, and bicyclic aromatic heterocycles containing from eight to twelve ring members, of which from 1 to 4 can be heteroatoms selected, independently, from N, S and O.

The term “one or more substituents”, as used herein, refers to a number of substituents that equals from one to the maximum number of substituents possible based on the number of available bonding sites.

The terms “halo” and “halogen”, as used herein, unless otherwise indicated, include, fluoro, chloro, bromo and iodo.

The term “treating”, as used herein, refers to reversing, alleviating, inhibiting the progress of, or preventing the disorder or condition to which such term applies, or preventing one or more symptoms of such condition or disorder.

The term “treatment”, as used herein, refers to the act of treating, as “treating” is defined immediately above.

The compounds of formula 1 and the Group A compounds, and the pharmaceutically acceptable salts of these compounds are referred to herein, collectively, as the “novel compounds of this invention” and the “active compounds of this invention”.

This invention also relates to a pharmaceutical composition comprising a therapeutically effective amount of a compound of the formula 1, or a Group A compound, or a pharmaceutically acceptable salt thereof, and a pharmaceutically acceptable carrier.

Compounds of the formula 1 and the Group A compounds may contain chiral centers and therefore may exist in different enantiomeric and diastereomeric forms. This invention relates to all optical isomers and all stereoisomers of compounds of the formula 1 and the Group A compounds, both as racemic mixtures and as individual enantiomers and diastereoisomers of such compounds, and mixtures thereof, and to all pharmaceutical compositions and methods of treatment defined above that contain or employ them, respectively. Individual isomers can be obtained by known methods, such as optical resolution, fractional crystallization, optically selective reaction, or chromatographic separation in the preparation of the final product or its intermediate. Individual enantiomers of the compounds of formula 1 and the Group A compounds may have advantages, as compared with the racemic mixtures of these compounds, in the treatment of various disorders or conditions.

In so far as the compounds of formula 1 and the Group A compounds are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the base compounds of this invention are readily prepared by treating the base compound with a substantially equivalent amount of the chosen mineral or organic acid in an aqueous solvent or in a suitable organic solvent, such as methanol or ethanol. Upon careful evaporation of the solvent, the desired solid salt is readily obtained. The acids which are used to prepare the pharmaceutically acceptable acid addition salts of the aforementioned base compounds of this invention are those which form non-toxic acid addition salts, i.e., salts containing pharmaceutically acceptable anions, such as the hydrochloride, hydrobromide, hydroiodide, nitrate, sulfate or bisulfate, phosphate or acid phosphate, acetate, lactate, citrate or acid citrate, tartrate or bi-tartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

The present invention also includes isotopically labelled compounds, which are identical to those of formula 1 and the Group A compounds, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula 1, the Group A compounds and prodrugs thereof can generally be prepared by carrying out the procedures disclosed in the Schemes and/or in the Examples below, by substituting a readily available isotopically labelled reagent for a non-isotopically labelled reagent.

The compounds of formula 1 and the Group A compounds have useful pharmaceutical and medicinal properties.

This invention also relates to a method of treating a disorder or condition selected from the group consisting of single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scoft syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising administering to a mammal in need of such treatment an amount of a compound of the formula 1 or a Group A compound, or a pharmaceutically acceptable salt thereof, that is effective in treating such disorder or condition.

The compounds of formula 1 and the Group A compounds, and their pharmaceutically acceptable salts are also referred to herein, collectively, as the “novel compounds of this invention” and the “active compounds of this invention”.

This invention also relates to a pharmaceutical composition comprising a therapeutically effective amount of a compound of the formula 1 or a Group A compound, or a pharmaceutically acceptable salt thereof, and a pharmaceutically acceptable carrier.

This invention also relates to a pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-indliced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal in need of such treatment, including a human, comprising an amount of a compound of the formula 1 or a Group A compound, or a pharmaceutically acceptable salt thereof, that is effective in treating such disorder or condition, and a pharmaceutically acceptable carrier.

A more specific embodiment of this invention relates to the above method wherein the disorder or condition that is being treated is selected from major depression, single episode depression, recurrent depression, child abuse induced depression, postpartum depression, dysthymia, cyclothymia and bipolar disorder.

Another more specific embodiment of this invention relates to the above method wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

Another more specific embodiment of this invention relates to the above method wherein the disorder or condition that is being treated is selected from autism, pervasive development disorder, and attention deficit hyperactivity disorder.

Another more specific embodiment of this invention relates to the above method wherein the disorder or condition that is being treated is selected from generalized anxiety disorder, panic disorder, obsessive-compulsive disorder, post-traumatic stress disorder, and phobias, including social phobia, agoraphobia, and specific phobias.

Another more specific embodiment of this invention relates to the above method wherein the disorder or condition that is being treated is selected from movement disorders such as akinesias, dyskinesias, including familial paroxysmal dyskinesias, spasticities, Tourette's syndrome, Scott syndrome, PALSYS and akinetic-rigid syndrome; and extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour.

Another more specific embodiment of this invention relates to the above method wherein the disorder or condition that is being treated is selected from delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorder, vascular dementia, and other dementias, for example, due to HIV disease, head trauma, Parkinson's disease, Huntington's disease, Pick's disease, Creutzfeldt-Jakob disease, or due to multiple etiologies.

Another more specific embodiment of this invention relates to the above method wherein the compound of formula 1 is administered to a human for the treatment of any two or more comorbid disorders or conditions selected from those disorders and conditions referred to in any of the above methods.

For the treatment of depression, anxiety, schizophrenia or any of the other disorders and conditions referred to above in the descriptions of the methods and pharmaceutical compositions of this invention, the novel compounds of this invention can be used in conjunction with one or more other antidepressants or anti-anxiety agents. Examples of classes of antidepressants that can be used in combination with the active compounds of this invention include norepinephrine reuptake inhibitors, selective serotonin reuptake inhibitors (SSRIs), NK-1 receptor antagonists, monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butripyline, iprindole, lofepramine, nortriptyline, protriptyline, amoxapine, desipramine and maprotiline. Suitable selective serotonin reuptake inhibitors include fluoxetine, fluvoxamine, paroxetine and sertraline. Examples of monoamine oxidase inhibitors include isocarboxazid, phenelzine, and tranylcyclopramine. Suitable reversible inhibitors of monoamine oxidase include moclobemide. Suitable serotonin and noradrenaline reuptake inhibitors of use in the present invention include venlafaxine. Suitable CRF antagonists include those compounds described in International Patent Application Nos. WO 94/13643, WO 94/13644, WO 94/13661, WO 94/13676 and WO 94/13677. Suitable atypical anti-depressants include bupropion, lithium, nefazodone, trazodone and viloxazine. Suitable NK-1 receptor antagonists include those referred to in World Patent Publication WO 01/77100.

Suitable classes of anti-anxiety agents that can be used in combination with the active compounds of this invention include benzodiazepines and serotonin 1A (5-HT_{1A}) agonists or antagonists, especially 5-HT_{1A} partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam. Suitable 5-HT_{1A} receptor agonists or antagonists include buspirone, flesinoxan, gepirone and ipsapirone.

This invention also relates to a method of treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal in need of such treatment, including a human, comprising administering to said mammal:

(a) a compound of the formula 1 or a Group A compound, or a pharmaceutically acceptable salt thereof; and

(b) another pharmaceutically active compound that is an antidepressant or anti-anxiety agent, or a pharmaceutically acceptable salt thereof;

wherein the active compounds “a” and “b” are present in amounts that render the combination effective in treating such disorder or condition.

A more specific embodiment of this invention relates to the above method wherein the disorder or condition that is being treated is selected from major depression, single episode depression, recurrent depression, child abuse induced depression, postpartum depression, dysthymia, cyclothymia and bipolar disorder.

Another more specific embodiment of this invention relates to the above method wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

Another more specific embodiment of this invention relates to the above method wherein the disorder or condition that is being treated is selected from autism, pervasive development disorder, and attention deficit hyperactivity disorder.

Another more specific embodiment of this invention relates to the above method wherein the disorder or condition that is being treated is selected from generalized anxiety disorder, panic disorder, obsessive-compulsive disorder, post-traumatic stress disorder, and phobias, including social phobia, agoraphobia, and specific phobias.

Another more specific embodiment of this invention relates to the above method wherein the disorder or condition that is being treated is selected from movement disorders such as akinesias, dyskinesias, including familial paroxysmal dyskinesias, spasticities, Tourette's syndrome, Scott syndrome, PALSYS and akinetic-rigid syndrome; and extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour.

Another more specific embodiment of this invention relates to the above method wherein the disorder or condition that is being treated is selected from delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorder, vascular dementia, and other dementias, for example, due to HIV disease, head trauma, Parkinson's disease, Huntington's disease, Pick's disease, Creutzfeldt-Jakob disease, or due to multiple etiologies.

Another more specific embodiment of this invention relates to the above method wherein the compound of formula 1 and the additional antidepressant or anti-anxiety agent are administered to a human for the treatment of any two or more comorbid disorders or conditions selected from those disorders and conditions referred to in any of the above methods.

This invention also relates to a pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal in need of such treatment, including a human, comprising:

(a) a compound of the formula 1 or a Group A compound, or a pharmaceutically acceptable salt thereof;

(b) another pharmaceutically active compound that is an antidepressant or anti-anxiety agent, or a pharmaceutically acceptable salt thereof; and

(c) a pharmaceutically acceptable carrier;

wherein the active compounds “a” and “b” are present in amounts that render the composition effective in treating such disorder or condition.

### DETAILED DESCRIPTION OF THE INVENTION

The active compounds of this invention may be prepared as described in the following reaction schemes. Unless otherwise indicated, A, W¹, W², X, R and R¹ through R¹¹ in the reaction schemes and discussion that follow, are as defined above.

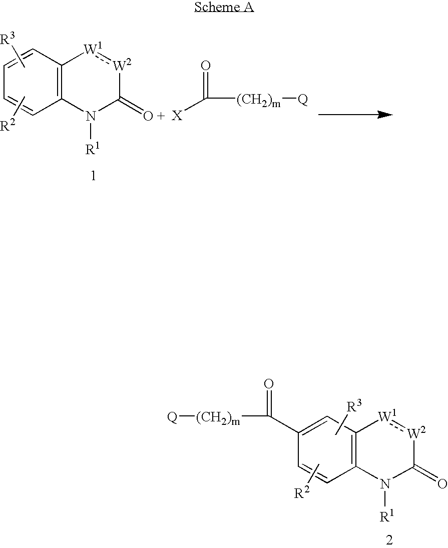

Scheme A illustrates a method for preparing compounds of the formula 2 by reacting a compound of the formula 1 with a compound of formula XCO(CH₂)ₘQ, wherein m is an integer from 1 to four, X is either a halogen or OH and Q is either a halogen, mesylate, or tosylate. When X is represented by a halogen, the reaction is typically carried out in the presence of a Lewis acid such as aluminum bromide (AlBr₃), aluminum chloride (AlCl₃), gallium trichloride (GaCl₃), ferric chloride (FeCl₃), zinc chloride (ZnCl₂), antimony pentachloride (SbCl₅), zirconium tetrachloride (ZrCl₄), tin tetrachloride (SnCl₄), boron trichloride (BCl₃), boron trifluoride (BF₃), or antimony trichloride (SbCl₃). The reaction can be carried out in nonpolar solvents such as chloroform, dichloromethane, or carbon disulfide, or in polar solvents such as nitrobenzene, or may be run neat in the presence of excess Lewis acid. The reaction is typically carried out at a temperature of 25° C. to about 120° C. for a period of about 1 hour to 6 hours. Where X is represented by OH, the reaction is typically carried out in the presence of a proton acid such as polyphosphoric acid or sulfuric acid.

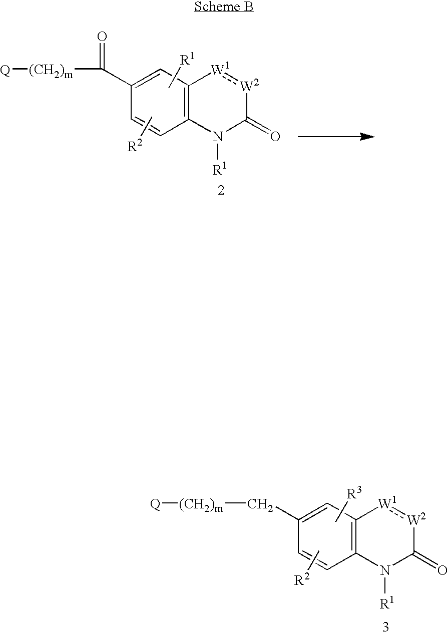

Scheme B illustrates a method for preparing compounds of the formula 3. In compounds of the formulas 2 and 3, Q and m are defined as they are defined above in the description of Scheme 1. The reaction illustrated in Scheme B can be carried out using triethylsilane in trifluoroacetic acid at a temperature from about room temperature to the reflux temperature of the solvent for a period of up to about 24 hours. Alternatively, the reaction may be carried out using borane-tert-butylamine in the presence of a Lewis acid such as aluminum chloride or by using borane-dimethylamine in the presence of a Lewis acid such as titanium tetrachloride in an inert solvent such as dichloromethane, chloroform, or nitrobenzene under temperatures described.

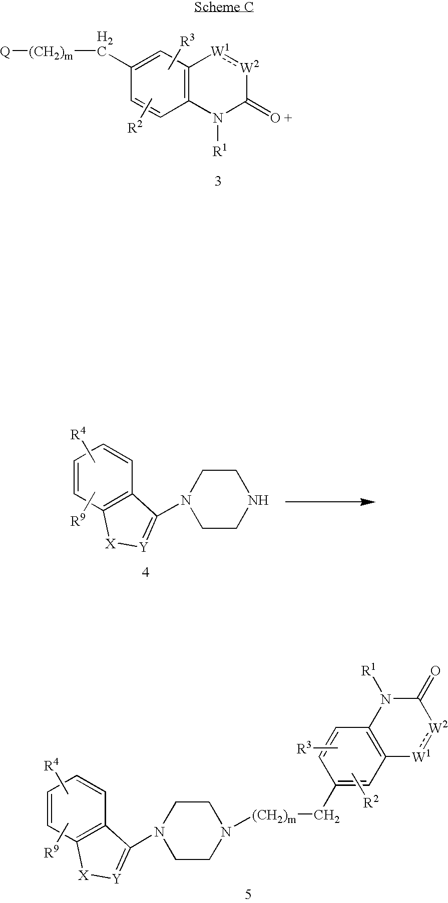

Scheme C illustrates a method for preparing compounds of the formula 5, which include the Group A compounds, by reacting a compound of the formula 3, as described in scheme B, with a compound of formula 4. The reaction is typically run in the presence of a base such as potassium carbonate, sodium carbonate, triethylamine, or diisopropylethylamine. The solvent used may be water, acetonitrile, dioxane, benzene, toluene, tetrahydrofuran, methyl isobutyl ketone, or a combination of two of the formerly mentioned solvents. Inorganic salts such as a sodium or potassium halide (e.g., sodium iodide or potassium iodide) may be employed as catalysts in the reaction. The temperature of the reaction may vary from ambient to reflux temperature of the solvent used, preferably from about 80° C. to 120° C., for a period of about 1 hour to about 96 hours, preferably from about 12 hours to 48 hours.

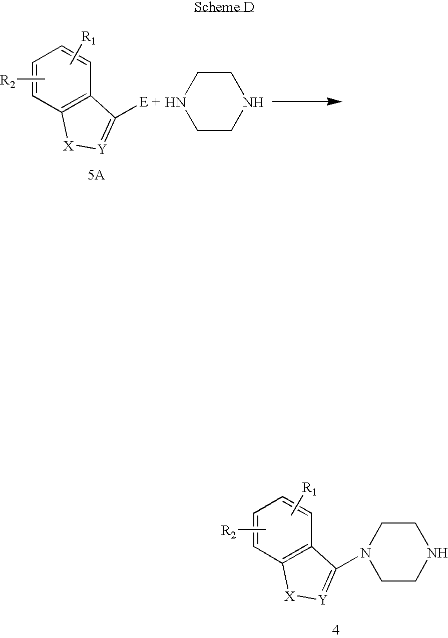

Scheme D illustrates a method for preparing compounds of the formula 4 (from Scheme C) by reacting a compound of the formula 5A, wherein E is either bromine, chlorine, tosylate, mesylate, or triflate, with an excess of piperazine (preferably 5 to 6 molar equivalents in relation to 5). The reaction is typically run neat in a sealed vessel at a temperature ranging from 100° C. to 250° C., preferably around 200° C., for a period of about 1 hour to 30 hours, preferably from about 12 to 24 hours. The use of a catalyst such as copper (bronze), tin, or iron filings, may be employed. Alternatively, the reaction may be run in the presence of a base such as sodium carbonate, potassium carbonate, or sodium bicarbonate with a catalyst such as sodium iodide or potassium iodide in a solvent such as acetonitrile, dioxane, toluene, or xylene. Under such conditions the temperature of the reaction may vary depending on the reflux temperature of the solvent used, and is preferably from about 80° C. to 140° C. The reaction is typically carried out for a period of about 1 hour to about 96 hours, preferably from about 12 hours to about 48 hours.

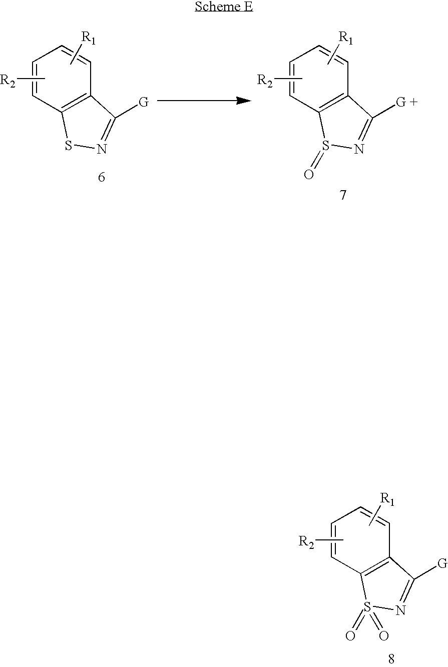

Scheme E illustrates a method for preparing cyclic sulfinamides of the formula 7 and cyclic sulfonamides of the formula 8 from benzisothiazoles of the formula 6, wherein G is chlorine, bromine, or piperazin-1-yl. The reaction is run in the presence of an oxidizing agent such as H₂O₂, CrO₃, NaIO₄, t-BuOCl, sodium perborate, peracids (i.e. m-chloroperbenzoic acid, peracetic acid), potassium hydrogen persulfate, formic acid, KNO₃ or HNO₃ in sulfuric acid, and acyl nitrites at a range of temperature of −10° C. to 100° C., but preferably at around −10° C. to 40° C. The reaction is typically run for a period of 1 hour up to 48 hours but preferably between 4 and 12 hours. If enough oxidizing agent is used, compounds of the formula 8 may be obtained exclusively from compounds of the formula 6 or 7.

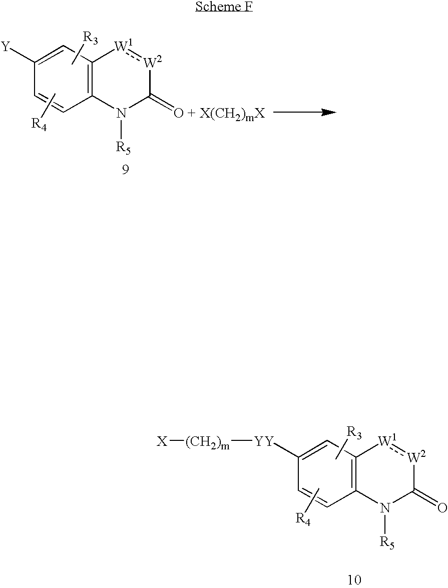

Scheme F illustrates a method for preparing compounds of the formula 10 by reacting compounds of the formula 9 with compounds of the formula X(CH₂)ₘX, wherein X is either bromine or chlorine, m is an integer from one to four and Y is either OH or NHR¹¹ and YY is 0 or NR¹¹. Compounds of the formula 9 wherein Y is OH can be prepared as described in DE415096, U.S. Pat. No. 3,819,637, J. Chin. Chem. Soc. (Taipei), 2000, 47, 155, and Chem. Heterocyclic Compd., 1970, 6, 1283. Compounds of formula 9 wherein Y is NHR¹¹ can be prepared as described in J. Chem. Soc., C, 1969, 183, J. Med. Chem., 1989, 32, 1173, Chem. Ber., 1903, 36, 1175, and J. Chem. Res. Miniprint, 1997, 9, 2068. The above reaction is typically carried out in the presence of a base such as NaOH, KOH, K₂CO₃, NaH, NaOMe, or NaOEt using solvents such as tetrahydrofuran, ethanol, methanol, butan-2-one, methyl isobutyl ketone, acetone, or N,N-dimethylformamide. The reaction may be run at a temperature range from about ambient temperature to about the reflux temperature of the solvent used and is typically run for a period of about 1 hour to about 24 hours, preferably between about 4 and about 12 hours. Reaction of compounds of the formula 10 with compounds of the formula 4 (scheme D), in accordance with the methodology described in Scheme C, yields the corresponding compounds of the formula 5.

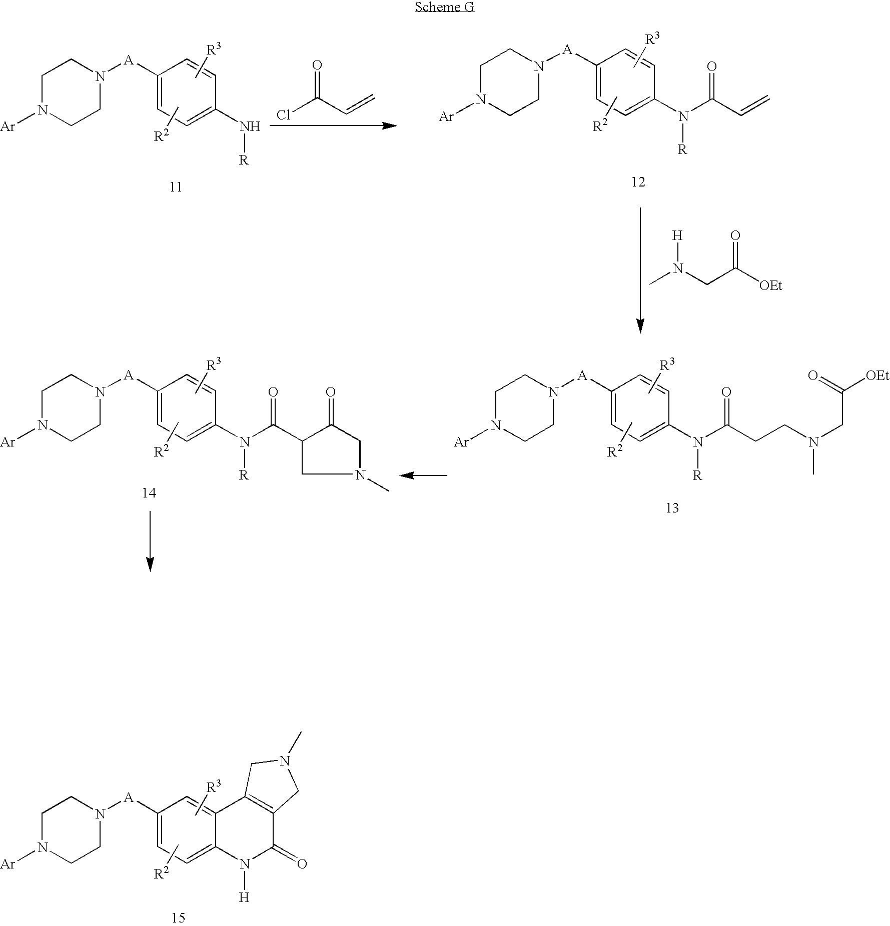

Referring to Scheme G, compounds of formula 11 can be converted to compounds of formula 12 by treatment with acryolyl chloride in the presence of a suitable base such as pyridine or triethylamine, preferably triethylamine, in a suitable solvent like pyridine, benzene, toluene, dichloroethane or dichloromethane, preferably dichloromethane, at a temperature from about 0° C. to about 60° C., preferably at room temperature, for about 30 minutes to about 24 hours, preferably for about 2 hours. Compounds of formula 13 can be prepared from compounds of formula 12 by treatment with methylamino-acetic acid ethyl ester in a mixture of methanol and triethylamine in the presence of 2,6-di-tert-butyl cresol at about the reflux temperature of the reaction mixture for about 24 hours. Compounds of formula 13 can be converted into compounds of formula 14 by treatment with a suitable base such as sodium methoxide or potassium t-butoxide, preferably potassium t-butoxide, in a suitable polar or ethereal solvent such as dimethylformamide (DMF), dioxane, diglyme or tetrahydrofuran (THF), preferably (THF), at a temperature of about 5-10° C. for about 4 hours. Compounds of formula 15 can be prepared from compounds of formula 14 by treatment with a strong mineral acid such as hydrochloric acid, sulfuric acid, or polyphosphoric acid, preferably polyphosphoric acid, at a temperature from about 100° C. to about 150° C., preferably at about 130° C., for a period from about one hour to about 10 hours, preferably for about 3 hours.

Compounds of formula 18 can be prepared from compounds of formula 16 as illustrated below in Scheme H.

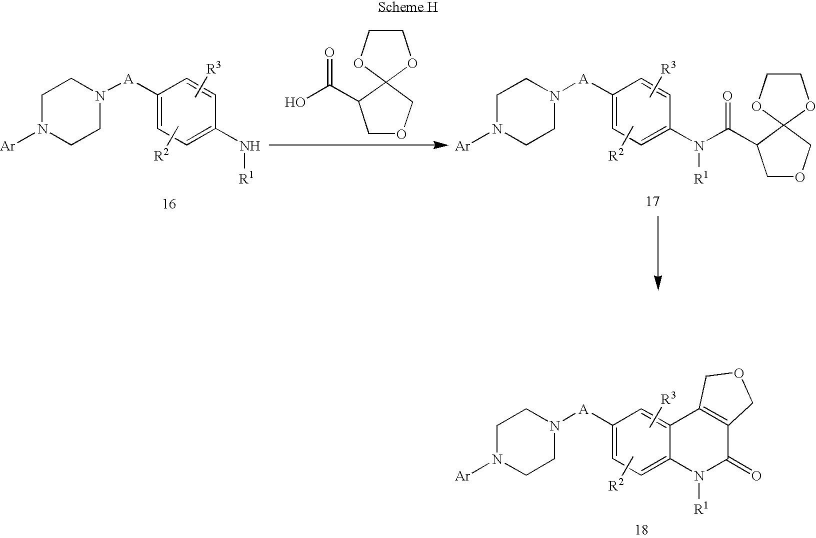

Referring to Scheme H, compounds of formula 17 can be made by treatment of compounds of formula 16 with 1,4,7-trioxa-spiro[4,4]nonane-9-carboxylic acid (the preparation of which is described in Example 168), preferably the acid chloride, which can be made by treatment of the corresponding acid with oxalyl chloride in dichloromethane, in the presence of a suitable base, such as pyridine, potassium carbonate, sodium carbonate, diisopropylethylamine or triethylamine, preferably triethylamine. Compounds of formula 18 can be prepared by treatment of compounds of formula 17 with a strong mineral acid such as hydrochloric acid, sulfuric acid, or polyphosphoric acid, at a temperature from about 0° C. to about 100° C. Preferably, this reaction is conducted using sulfuric acid at about 60° C. for a period of about 10 minutes to about 5 hours, preferably about 45 minutes.

The preparation of other compounds of the formula 1 and the Group A compounds not specifically described in the foregoing experimental section can be accomplished using combinations of the reactions described above that will be apparent to those skilled in the art.

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience.

The compounds of the formula 1 and the Group A compounds, and the intermediates shown in the above reaction schemes can be isolated and purified by conventional procedures, such as recrystallization or chromatographic separation.

The compounds of the formula 1 and the Group A compounds, and their pharmaceutically acceptable salts, can be administered to mammals via either the oral, parenteral (such as subcutaneous, intravenous, intramuscular, intrasternal and infusion techniques), rectal, buccal or intranasal routes. In general, these compounds are most desirably administered in doses ranging from about 3 mg to about 600 mg per day, in single or divided doses (i.e., from 1 to 4 doses per day), although variations will necessarily occur depending upon the species, weight and condition of the patient being treated and the patient's individual response to said medicament, as well as on the type of pharmaceutical formulation chosen and the time period and interval at which such administration is carried out. However, a dosage level that is in the range of about 25 mg to about 100 mg per day is most desirably employed. In some instances, dosage levels below the lower limit of the aforesaid range may be more than adequate, while in other cases still larger doses may be employed without causing any harmful side effects, provided that such higher dose levels are first divided into several small doses for administration throughout the day.

The novel compounds of the present invention may be administered alone or in combination with pharmaceutically acceptable carriers or diluents by any of the routes previously indicated, and such administration may be carried out in single or multiple doses. More particularly, the novel therapeutic agents of this invention can be administered in a wide variety of different dosage forms, i.e., they may be combined with various pharmaceutically acceptable inert carriers in the form of tablets, capsules, lozenges, troches, hard candies, suppositories, jellies, gels, pastes, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the weight ratio of the novel compounds of this invention to the pharmaceutically acceptable carrier will be in the range from about 1:6 to about 2:1, and preferably from about 1:4 to about 1:1.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intra-articular, intra-muscular and subcutaneous injection purposes. The preparation of all these solutions under sterile conditions is readily accomplished by standard pharmaceutical techniques well known to those skilled in the art.

This invention relates to methods of treating anxiety, depression, schizophrenia and the other disorders referred to in the description of the methods of the present invention, wherein a novel compound of this invention and one or more of the other active agents referred to above (e.g., an NK1 receptor antagonist, tricyclic antidepressant, 5HT1D receptor antagonist, or serotonin reuptake inhibitor) are administered together, as part of the same pharmaceutical composition, as well as to methods in which such active agents are administered separately as part of an appropriate dose regimen designed to obtain the benefits of the combination therapy. The appropriate dose regimen, the amount of each dose of an active agent administered, and the specific intervals between doses of each active agent will depend upon the subject being treated, the specific active agent being administered and the nature and severity of the specific disorder or condition being treated. In general, the novel compounds of this invention, when used as a single active agent or in combination with another active agent, will be administered to an adult human in an amount from about 3 mg to about 300 mg per day, in single or divided doses, preferably from about 25 to about 100 mg per day. Such compounds may be administered on a regimen of up to 6 times per day, preferably 1 to 4 times per day, especially 2 times per day and most especially once daily. Variations may nevertheless occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

A proposed daily dose of a 5HT reuptake inhibitor, preferably sertraline, in the combination methods and compositions of this invention, for oral, parenteral or buccal administration to the average adult human for the treatment of the conditions referred to above, is from about 0.1 mg to about 2000 mg, preferably from about 1 mg to about 200 mg of the 5HT reuptake inhibitor per unit dose, which could be administered, for example, 1 to 4 times per day. A proposed daily dose of a 5HT1D receptor antagonist in the combination methods and compositions of this invention, for oral, parenteral, rectal or buccal administration to the average adult human for the treatment of the conditions referred to above, is from about 0.01 mg to about 2000 mg, preferably from about 0.1 mg to about 200 mg of the 5HT1D receptor antagonist per unit dose, which could be administered, for example, 1 to 4 times per day.

For intranasal administration or administration by inhalation, the novel compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch. Formulations of the active compounds of this invention for treatment of the conditions referred to above in the average adult human are preferably arranged so that each metered dose or “puff” of aerosol contains 20 μg to 1000 μg of active compound. The overall daily dose with an aerosol will be within the range 100 μg to 10 mg. Administration may be several times daily, for example 2, 3, 4 or 8 times, giving for example, 1, 2 or 3 doses each time.

All of the title compounds of the examples were tested and at least one stereoisomer of each such compound exhibited a binding affinity for the D2 receptor, measured as percent inhibition at a concentration of 0.1 μm, of no less than 14% and up to 100%. At least one stereoisomer of each such compound exhibited a binding affinity for the 5HT2 receptor, measured as percent inhibition at a concentration of 0.1 μm, of no less than 80% and up to 100%.

The ability of the novel compounds of this invention to bind to the dopamine D2 and serotonin 2A (5HT2A) receptors can be determined using conventional radioligand receptor binding assays. All receptors can be heterologously expressed in cell lines and experiments conducted in membrane preparations from the cell lines using procedures outlined below. IC₅₀ concentrations can be determined by nonlinear regression of concentration-dependent reduction in specific binding. The Cheng-Prussoff equation can be used to convert the IC₅₀ to Ki concentrations.

Dopamine D2Receptor Binding:

[³H]Spiperone binding to a membrane preparation from CHO-hD2L cells is carried out in 250 μl of 50 mM Tris-HCl buffer containing 100 mM NaCl, 1 mM MgCl₂ and 1% DMSO at pH 7.4. Duplicate samples containing (in order of addition) the test compounds, 0.4 nM [³H]spiperone and approximately 12 μg protein are incubated for 120 minutes at room temperature. Bound radioligand is separated by rapid filtration under reduced pressure through Whatman GF/B glass fiber filters previously treated with 0.3% polyethyleneimine. Radioactivity retained on the filter is determined by liquid scintillation spectrophotometry.

The title compounds of Examples 1-36 were tested using the above assay, in which specific binding determined in the presence of 1 mM haloperidol was 95%. All of the title compounds of Examples 1-36 exhibited Ki values less than or equal to 1 uM. The title compound of Example 8 exhibited a Ki of 7 nM. The title compound of Example 31 exhibited a Ki of 1 nM. The title compound of Example 23 exhibited a Ki of 0.9 nM.

Serotonin 2A Binding:

[³H]Ketanserin binding to Swiss-h5HT2A cell membranes can be carried out in 250 μl of 50 mM Tris-HCl buffer pH 7.4. Duplicate samples containing (in order of addition) test compounds, 1.0 nM [³H]ketanserin, and approximately 75 μg protein are incubated for 120 minutes at room temperature. Bound radioligand is separated by rapid filtration under reduced pressure through Whatman GF/B glass fiber filters previously treated with 0.3% polyethyleneimine. Radioactivity retained on the filter is determined by liquid scintillation spectrophotometry.

The title compounds of Examples 1-36 were tested using the above assay, in which specific binding determined in the presence of 1 mM ketanserin was 90%. All of the title compounds of Examples 1-36 exhibited Ki values less than or equal to 1 uM. The title compound of Example 8 exhibited a Ki of 5 nM. The title compound of Example 31 exhibited a Ki of 2 nM. The title compound of Example 23 exhibited a Ki of 1 nM.

The following Examples illustrate the preparation of the compounds of the present invention. Melting points are uncorrected. NMR data are reported in parts per million and are referenced to the deuterium lock signal from the sample solvent.

### EXAMPLES

### Example 1

### 6-[2-(4-BENZO[D]ISOXAZOL-3-YL-PIPERAZIN-1-YL)-ETHYL]-4-METHYL-3,4-DIHYDRO-1H-QUINOLIN-2-ONE

A. 6-(2-Chloroacetyl)-4-methyl-3,4-dihydro-1H-quinolin-2-one

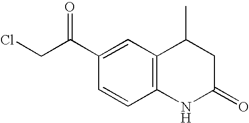

4-Methyl-3,4-dihydro-1H-quinolin-2-one (4.38 g, 0.027 mol, prepared according to the procedure in J. Org. Chem., 1958, 23, 1330) was added to a mixture of aluminum chloride (16.68 grams (g), 0.125 mol) and chloroacetyl chloride (3.58 ml, 0.045 mol) in carbon disultide (190 ml) with vigorous stirring. The reaction mixture was refluxed for 2 hours and cooled to room temperature. The solvent was decanted and the residue was treated with cold water under vigorous agitation. The precipitate was collected and washed with water to give 6.29 g (98%). MS (APCI): (M+1)⁺=238.

B. 6-(2-Chloroethyl)-4-methyl-3,4-dihydro-1H-quinolin-2-one

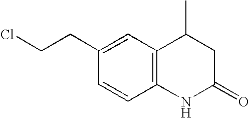

To a mixture of the product from step A (6.29 g, 0.026 mol) and trifluoroacetic acid (20 ml, 0.26 mol), cooled to 0° C. under nitrogen, was added portionwise triethylsilane (9.57 ml, 0.059 mol). The reaction mixture was heated at 40-45° C. for 20 minutes and then stirred at room temperature for 16 hours. The solution was poured into ice water layered with hexane and vigorously stirred for several hours. The precipitate that formed was collected and washed with water and hexanes to give 4.51 g (78%). MS (APCI): (M +1)⁺=224.

C. 6-[2-(4-Benzo[d]isoxazol-3-yl-piperazin-1-yl)-ethyl]-4-methyl-3,4-dihydro-1H-quinolin-2-one

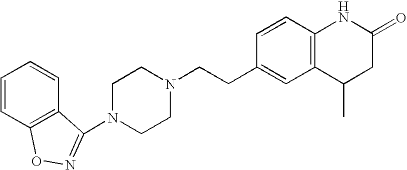

A mixture of the product from step B (1.61 g, 7.20 mmol), 3-piperazin-1-yl-benzo[d]isoxazole hydrochloride (1.15 g, 4.80 mmol, prepared according to J. Med. Chem., 1986, 29, 359), sodium carbonate (1.12 g, 10.5 mmol) and sodium iodide (150 mg) in 1:1 (v/v) water:1,4-dioxane (60 ml) was refluxed for 44 hours with vigorous stirring. The reaction mixture was concentrated and the residue was partitioned between water and methylene chloride. The organic layer was dried over magnesium sulfate, filtered, and concentrated. The crude product was purified by elution through a flash column (silica gel 60, 230-400 mesh, ethyl acetate) to give a white, crystalline solid which was washed with acetone upon collection, yield=744 mg (40%). MS (APCI): (M+1)⁺=391, (M−1)⁺=389. ¹H-NMR (DMSO-d₆, δ): 9.99 (s, 1H), 7.95 (d, 1H, J=8.1 Hz), 7.54 (d, 2H, J=3.7 Hz), 7.25 (m, 1H), 7.05 (s, 1H) 6.98 (d, 1H, J=6.1 Hz), 6.73 (d, 1H, J=8.1 Hz), 3.45 (t, 4H, J=4.6, 5.1 Hz), 2.98 (q, 1H, J=7.1, 6.4, 6.8 Hz), 2.66 (t, 2H, J=3.4, 5.1 Hz), 2.60 (t, 4H, J=4.9, 4.9 Hz), 2.51 (m, 3H), 2.17 (dd, 1H, J=7.1, 7.1 Hz), 1.13 (d, 3H, J=6.8 Hz). TLC: R_{f}=0.21 ethyl acetate (EtOAc). CHN: Calculated for C₂₃H₂₆N₄O₂, C: 70.75%, H: 6.71%, N: 14.35%; found, C: 70.80%, H: 6.67%, N: 14.15%. HPLC: Chiralpak AD, 250×4.6 mm; mobile phase, 10% ethanol (EtOH) in hexane; flow rate, 0.50 ml/min; peak 1: RT=35.19 min (52%), peak 2: RT=38.72 min (48%).

The procedure described for the preparation of step C of Example 1 was used to prepare the title compounds of Examples 2 and 3.

### Example 2

### 6-[2-(4-BENZO[D]ISOXAZOL-3-YL-PIPERAZIN-1-YL)-ETHYL]-4S-METHYL-3,4-DIHYDRO-1H-QUINOLIN-2-ONE

The title compound was prepared from 3-piperazin-1-yl-benzo[d]isoxazole (1.0 g, 4.17 mmol) and 6-(2-chloroethyl)-4S-methyl-3,4-dihydro-1H-quinolin-2-one (1.40 g, 6.26 mmol, prepared according to U.S. Pat. No. 5,350,747 Sep. 27, 1994) to give, after purification, 517 mg (32%). MS (APCI): (M+1)⁺=391; (M−1)⁺=389. ¹H-NMR (DMSO-d₆, δ): 9.99 (s, 1H), 7.95 (d, 1H, J=8.1 Hz), 7.54 (d, 2H, J=3.7 Hz), 7.25 (m, 1H), 7.05 (s, 1H) 6.98 (d, 1H, J=6.1 Hz), 6.73 (d, 1H, J=8.1 Hz), 3.45 (t, 4H, J=4.6, 5.1 Hz), 2.98 (q, 1H, J=7.1, 6.4, 6.8 Hz), 2.66 (t, 2H, J=3.4, 5.1 Hz), 2.60 (t, 4H, J=4.9, 4.9 Hz), 2.51 (m, 3H), 2.17 (dd, 1H, J=7.1, 7.1 Hz), 1.13 (d, 3H, J=6.8 Hz). TLC: R_{f}=0.22 (EtOAc). CHN: Calculated for C₂₃H₂₆N₄O₂, C: 70.75%, H: 6.71%, N: 14.35%; found, C: 70.54%, H: 6.74%, N: 14.25%. HPLC: ChiralCel OD-H, 5 um, 250×4.6 mm; mobile phase, 20% isopropylalcohol (IPA) in hexane; flow rate, 0.30 ml/min; peak RT=63.07 min (98.66%). Optical Rotation: [α]_{D}²⁵=+4° (MeOH, c=11.4 mg/ml).

### Example 3

### 6-[2-(4-BENZO[D]ISOXAZOL-3-YL-PIPERAZIN-1-YL)-ETHYL]-4R-METHYL-3,4-DIHYDRO-1H-QUINOLIN-2-ONE

The title compound was prepared from 3-piperazin-1-yl-benzo[d]isoxazole (1.0 g, 4.17 mmol) and 6-(2-chloroethyl)-4R-methyl-3,4-dihydro-1H-quinolin-2-one (1.40 g, 6.26 mmol, prepared according to U.S. Pat. No. 5,350,747 Sep. 27, 1994) to give, after purification, 443 mg (27%). MS (APCI): (M+1)⁺=391; (M−1)⁺=389. ¹H-NMR (DMSO-d₆, δ): 9.99 (s, 1H), 7.95 (d, 1H, J=8.1 Hz), 7.54 (d, 2H, J=3.7 Hz), 7.25 (m, 1H), 7.05 (s, 1H) 6.98 (d, 1H, J=6.1 Hz), 6.73 (d, 1H, J=8.1 Hz), 3.45 (t, 4H, J=4.6, 5.1 Hz), 2.98 (q, 1H, J=7.1, 6.4, 6.8 Hz), 2.66 (t, 2H, J=3.4, 5.1 Hz), 2.60 (t, 4H, J=4.9, 4.9 Hz), 2.51 (m, 3H), 2.17 (dd, 1H, J=7.1, 7.1 Hz), 1.13 (d, 3H, J=6.8 Hz). TLC: R_{f}=0.20 (EtOAc). CHN: Calculated for C₂₃H₂₆N₄O₂, C: 70.75%, H: 6.71%, N: 14.35%; found, C: 70.35%, H: 6.83%, N: 14.20%. HPLC: ChiralCel OD-H, 5 um, 250×4.6 mm; mobile phase, 20% IPA in hexane; flow rate, 0.30 ml/min; peak RT=73.81 min (98.53%). Optical Rotation: [α]_{D}²⁵=−6° (MeOH, c=7.1 mg/ml).

### Example 4

### 6-[2-(4-BENZO[D]ISOXAZOL-3-YL-PIPERAZIN-1-YL)-ETHYL]-1,4-DIMETHYL-3,4-DIHYDRO-1H-QUINOLIN-2-ONE

A. 1,4-Dimethyl-3,4-dihydro-1H-quinolin-2-one

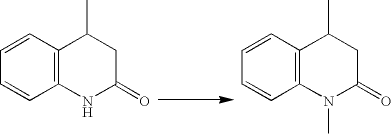

To a solution of 4-methyl-3,4-dihydro-1H-quinolin-2-one (4.0 g, 0.025 mol, prepared according to the procedure in J. Org. Chem., 1958, 23, 1330) in anhydrous tetrahydrofuran (THF) (60 ml), cooled to 0° C. under nitrogen, was added slowly with vigorous stirring sodium hydride (NaH) (60% dispersion in mineral oil, 1.12 g, 0.05 mol). After addition was complete the reaction mixture was stirred for 10 minutes and iodomethane (3.08 ml, 0.05 mol) was added. The reaction mixture was stirred at room temperature for 2 hours and quenched with water. The aqueous mixture was extracted with methylene chloride and the organic extract was dried over magnesium sulfate, filtered, and concentrated to an oil which was used without further purification, yield=4.38 g (100%). MS (APCI): (M+1)⁺=176.

B. 6-(2-Chloroacetyl)-1,4-dimethyl-3,4-dihydro-1H-quinolin-2-one

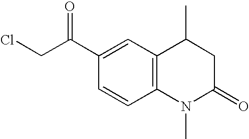

The title compound was prepared from the compound prepared in Step A (4.38 g, 0.025 mol) and chloroacetyl chloride (3.58 ml, 0.045 mol) based on the method described in step A of Example 1 to give 6.29 g (100%) of a brownish-grey solid. MS (APCI): (M+1)⁺=252; (M−1)⁺=250.

C. 6-(2-Chloroethyl)-1,4-dimethyl-3,4-dihydro-1H-quinolin-2-one

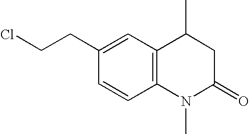

The title compound was prepared from the compound step B above (6.29 g, 0.025 mol) based on the procedure described in step B of Example 1 to give 4.51 g (76%) as an orange oil which crystallized on standing. MS (APCI): (M+1)⁺=238; (M+3)⁺240.

D. 6-[2-(4-Benzo[d]isoxazol-3-yl-piperazin-1-yl)-ethyl]-1,4-dimethyl-3,4-dihydro-1H-quinolin-2-one hydrochloride

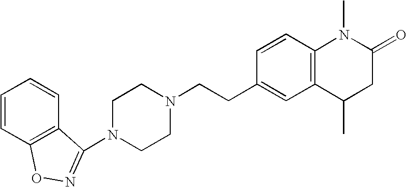

The title compound was prepared from 3-piperazin-1-yl-benzo[d]isoxazole hydrochloride (400 mg, 1.66 mmol) and the compound prepared in step C of Example 4 (592 mg, 2.49 mmol) based on the procedure described in step C of Example 1. The crude product was eluted through a flash column (silica gel 60, 230-400 mesh, EtOAc) to give an oil which was taken up in anhydrous diethyl ether and the solution treated with 4.0N hydrochloric acid (HCl) in dioxane to precipitate the hydrochloride salt, yield=281 mg (38%). MS (APCI): (M+1)⁺=405. ¹H-NMR (DMSO-d₆, δ): 11.10 (br s, 1H), 8.01 (d, 1H, J=8.1 Hz), 7.58 (t, 2H, J=1.9, 3.2 Hz), 7.30 (m, 1H), 7.12 (s, 2H), 7.02 (d, 1H, J=8.1 Hz), 4.11 (br d, 2H, J=13.7 Hz), 3.62 (br d, 2H, J=12 Hz), 3.50 (dd, 2H, J=6.1, 11.7 Hz), 3.32 (br s, 3H), 3.26 (m, 4H), 3.01 (m, 3H), 2.59 (dd, 1H, J=5.4, 5.6 Hz), 2.29 (dd, 1H, J=7.3, 7.1 Hz), 1.13 (d, 3H, J=6.8 Hz). TLC: R_{f}=0.20 (free base, EtOAc). CHN: Calculated for C₂₄H₂₈N₄O₂ 1.1 HCl, C: 64.83%, H: 6.60%, N: 12.60%; found, C: 64.62%, H: 6.52%, N: 12.05%.

Example 5

### 6-[2-(4-BENZO[D]ISOXAZOL-3-YL-PIPERAZIN-1-YL)-ETHYL]-4,4-DIMETHYL-3,4-DIHYDRO-1H-QUINOLIN-2-ONE

A. 3-Methyl but-2-enoic Acid Phenylamide

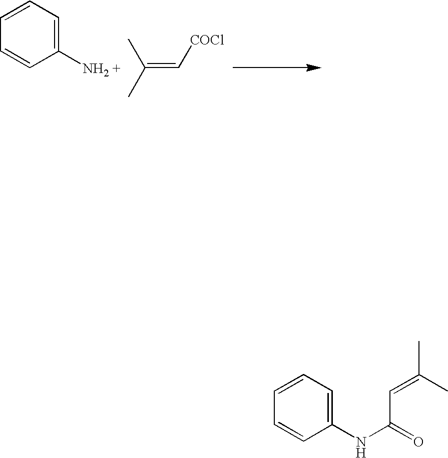

To a solution of aniline (219.78 g, 2.36 mol) in 3 L anhydrous chloroform at room temperature was added dropwise a solution of 3-methyl-but-2-enoyl chloride (301.14 g, 2.54 mol, Aldrich) in 500 milliliters (ml) chloroform (CHCl₃). After addition was complete the reaction mixture was filtered and the filtrate was washed with 1.0N aqueous HCl, dried over magnesium sulfate, filtered, and concentrated to an oil which solidified on standing, yield=235.89 g (53%). MS (APCI): (M+1)⁺=176.

B. 4,4-Dimethyl-3,4-dihydro-1H-quinolin-2-one

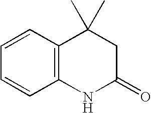

The compound prepared in step A above (234.80 g, 1.34 mol) was heated to 120° C. in an oil bath and portions of aluminum chloride were added (excess). The reaction was monitored by TLC and at total reaction of starting material heating was stopped. Upon cooling to room temperature, 3 liters (L) of methylene chloride was added to make a solution. The organic mixture was slowly treated with water, under vigorous agitation, until thorough quenching was achieved. The organic layer was separated, dried over magnesium sulfate, filtered and concentrated. The crude product was chromatographed (SiO₂, 9:1 hexanes:EtOAc) to give 98 g (42%). MS (APCI): (M+1)⁺=176.

C. 6-(2-Chloroacetyl)-4,4-dimethyl-3,4-dihydro-1H-quinolin-2-one

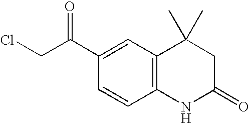

The compound prepared in step B above (73.6 g, 0.42 mol) underwent a Friedel-Crafts acylation according to the procedure described for example 2 to give 96.2 g (91%) product. MS (APCI): (M+1)⁺=252.

D. 6-(2-Chloroethyl)-4,4-dimethyl-3,4-dihydro-1H-quinolin-2-one

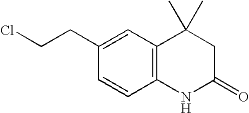

The compound prepared in step C above (3.0 g, 0.012 mol) underwent reduction of the ketone according to the procedure described in step B of Example 1 to give 2.09 g (73%) product. MS (APCI): (M+1)⁺=238; (M+3)⁺=240; (M−1)⁺=236.

E. 6-[2-(4-Benzo[d]isoxazol-3-yl-piperazin-1-yl)-ethyl]-4,4-dimethyl-3,4-dihydro-1H-quinolin-2-one

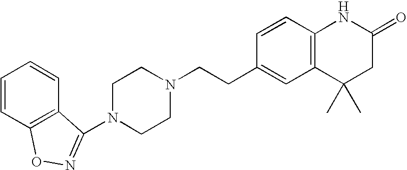

3-Piperazin-1-yl-benzo[d]isoxazole hydrochloride (1.0 g, 4.17 mmol) was reacted with the material prepared in step D above (1.49 g, 6.26 mmol) according to the procedure used in step C of Example 1 to give, after purification, 304 mg (18%) of an oil which crystallized on standing. MS (APCI): (M+1)⁺=405; (M−1)⁺=403. ¹H-NMR (DMSO-d₆, δ): 10.02 (s, 1H), 7.96 (d, 1H, J=7.8 Hz), 7.54 (d, 2H, J=3.9 Hz), 7.26 (m, 1H), 7.14 (s, 1H), 6.99 (d, 1H, J=7.8 Hz), 6.74 (d, 1H, J=8.1 Hz), 3.45 (br s, 4H), 2.69 (m, 2H), 2.61 (br s, 4H), 2.54 (m, 2H), 2.29 (s, 2H), 1.18 (s, 6H). TLC: R_{f}=0.27 (EtOAc). CHN: calculated for C₂₄H₂₈N₄O₂, C: 71.26%, H: 6.98%, N: 13.85%; found, C: 70.86%, H: 7.10%, N: 13.65%.

### Example 6

### 6-[2-(4-BENZO[D]ISOXAZOL-3-YL-PIPERAZIN-1-YL)-ETHYL]-1,4,4-TRIMETHYL-3,4-DIHYDRO-1H-QUINOLIN-2-ONE

A. 1,4,4-Trimethyl-3,4-dihydro-1H-quinolin-2-one

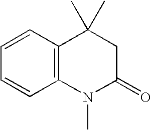

The compound prepared in step B of Example 5 (2.0 g, 0.0114 mol) was reacted according to the procedure described for example 6 to give, after purification, 1.63 g (76%) of an oil. MS (APCI): (M+1)⁺=190.

B. 6-(2-Chloroacetyl)-1,4,4-trimethyl-3,4-dihydro-1H-quinolin-2-one

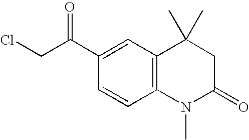

The compound prepared in step A above (1.63 g, 8.61 mmol) underwent a Friedel-Crafts acylation as described in example 1 to give 2.29 g (100%) of an oil which slowly solidified. MS (APCI): (M+1)⁺=266; (M−1)⁺=264.

C. 6-(2-Chloroethyl)-1,4,4-trimethyl-3,4-dihydro-1H-quinolin-2-one

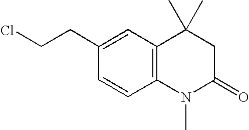

The ketone of step B above (2.29 g, 8.61 mmol) was reduced according to the procedure described in example 2 to give, after flash column purification, 1.88 g (87%) of an oil which crystallized on standing. MS (APCI): (M+1)⁺=252; (M+3)⁺=254.

D. 6-[2-(4-Benzo[d]isoxazol-3-yl-piperazin-1-yl)-ethyl]-1,4,4-trimethyl-3,4-dihydro-1H-quinolin-2-one Hydrochloride

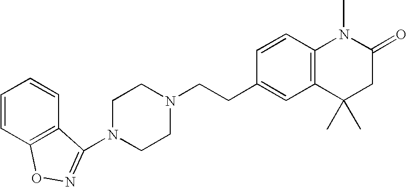

3-Piperazin-1-yl-benzo[d]isoxazole hydrochloride (400 mg, 1.66 mmol) was reacted with the compound prepared in step C above in accordance with the preparation described in example 3 to give, after flash column purification, a clear oil. The oil was taken up in anhydrous diethyl ether and the solution treated with 4.0N HCl in dioxane to precipitate 255 mg (34%) of the hydrochloride salt. MS (APCI): (M+1)⁺=419. ¹H-NMR (DMSO-d₆, δ): 11.0 (br s, 1H), 8.03 (d, 1H, J=8.1 Hz), 7.61 (s, 2H), 7.33 (m, 1H), 7.22 (s, 1H), 7.16 (d, 1H, J=8.5 Hz), 7.06 (d, 1H, J=8.3 Hz), 4.13 (br d, 2H, J=13.7 Hz), 3.64 (br d, 2H, J=12 Hz), 3.51 (m, 2H), 3,33 (br s, 3H), 3.28 (m, 4H), 3.05 (br s, 2H), 2.41 (s, 2H), 1.19 (s, 6H). TLC: R_{f}=0.35 (free base, EtOAc). CHN: calculated for C₂₅H₃₀N₄O₂ HCl, C: 65.99%, H: 6.87%, N: 12.31%; found, C: 65.62%, H: 6.89%, N: 12.23%.

### Example 7

### 6-[2-(4-BENZO[D]ISOXAZOL-3-YL-PIPERAZIN-1-YL)-ETHYL]-3-METHYL-3,4-DIHYDRO-1H-QUINOLIN-2-ONE

A. 6-(2-Chloroacetyl)-3-methyl-3,4-dihydro-1H-quinolin-2-one

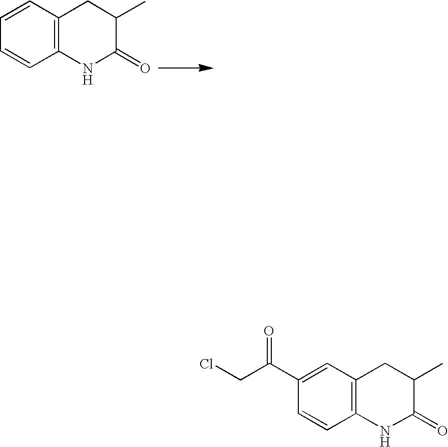

3-Methyl-3,4-dihydro-1H-quinolin-2-one was prepared according to the procedure described in J. Med. Chem., 1986, 29, 1832, and underwent a Friedel-Crafts acylation with chloroacetyl chloride in the manner described in step A of Example 1 to give the desired product as a solid. MS (APCI): (M+1)⁺=238.

B. 6-(2-Chloroethyl)-3-methyl-3,4-dihydro-1H-quinolin-2-one

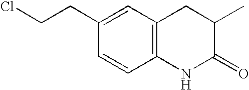

The product from step A above was treated with triethylsilane in trifluoroacetic acid according to the procedure described in step B of Example 1 to give the desired product as a solid. MS (APCI): (M+1)⁺=224.

C. 6-[2-(4-Benzo[d]isoxazol-3-yl-piperazin-1-yl)-ethyl]-3-methyl-3,4-dihydro-1H-quinolin-2-one

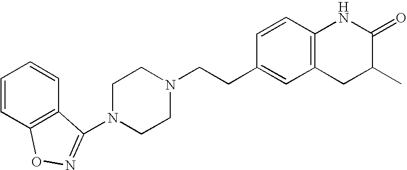

3-Piperazin-1-yl-benzo[d]isoxazole hydrochloride (500 mg, 2.08 mmol) was reacted with the compound described in step B above (699 mg, 3.13 mmol) using the procedure for example 3 to give the title compound, which precipitated out of solution as a white, crystalline solid, yield=371 mg (46%). MS (APCI): (M+1)⁺=391; (M−1)⁺=389. ¹H-NMR (DMSO-d₆, δ): 9.95 (s, 1H), 7.95 (d, 1H, J=8.1 Hz), 7.54 (d, 2H, J=3.7 Hz), 7.26 (m, 1H), 7.01 (s, 1H), 6.97 (d, 1H, J=8.1 Hz), 6.72 (d, 1H, J=8.1 Hz), 3.45 (t, 5H, J=4.6, 4.9 Hz), 2.85 (dd, 1H, J=5.9, 5.9 Hz), 2.66 (t, 2H, J=6.6, 8.8 Hz), 2.60 (t, 4H, J=4.2, 5.1 Hz), 2.53 (m, 3H), 1.07 (d, 3H, J=6.8 Hz). TLC: R_{f}=0.44 (1:9 MeOH:EtOAc). CHN: calculated for C₂₃H₂₆N₄O₂ 0.8 H₂O, C: 68.23%, H: 6.87%, N: 13.84%; found, C: 67.63%, H: 6.43%, N: 13.71%.

### Example 8

### 6-[2-(4-BENZO[D]ISOXAZOL-3-YL-PIPERAZIN-1-YL)-ETHYL]-3,3-DIMETHYL-3,4-DIHYDRO-1H-QUINOLIN-2-ONE

A. 6-(2-Chloroacetyl)-3,3-dimethyl-3,4-dihydro-1H-quinolin-2-one

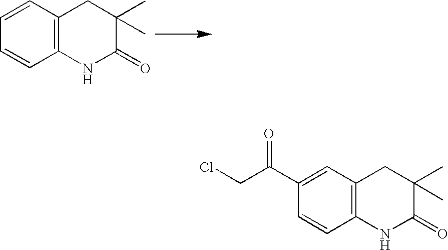

3,3-Dimethyl-3,4-dihydro-1H-quinolin-2-one was prepared according to the procedure in J. Med. Chem., 1986, 29, 1832, and underwent a Friedel-Crafts acylation with chloroacetyl chloride according to the procedure described in step A of Example 1 to give the title compound as a solid. MS (APCI): (M+1)⁺=252.

B. 6-(2-Chloroethyl)-3,3-dimethyl-3,4-dihydro-1H-quinolin-2-one

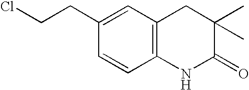

The compound prepared in step A above was treated with triethylsilane in trifluoroacetic acid according to the procedure described in step B of Example 1 to give the title compound as a solid. MS (APCI): (M+1)⁺=238.

C. 6-[2-(4-Benzo[d]isoxazol-3-yl-piperazin-1-yl)-ethyl]-3,3-dimethyl-3,4-dihydro-1H-quinolin-2-one

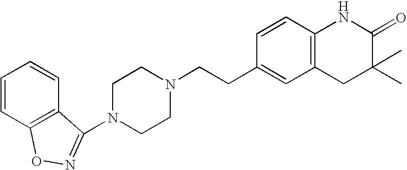

3-Piperazin-1-yl-benzo[d]isoxazole hydrochloride (500 mg, 2.08 mmol) was reacted with the compound prepared in step B above (743 mg, 3.13 mmol) according to the procedure given in step C of Example 1 to give the title compound, which precipitated out of solution as an off-white crystalline solid, yield=407 mg (48%). MS (APCI): (M+1)⁺=405; (M−1)⁺=403. ¹H-NMR (DMSO-d₆, δ): 9.91 (s, 1H), 7.95 (d, 1H, J=8.1 Hz), 7.54 (d, 2H, J=3.9 Hz), 7.26 (m, 1H), 6.98 (d, 2H, J=8.1 Hz), 6.72 (d, 2H, J=7.8 Hz), 3.45 (t, 4H, J=4.4, 5.1 Hz), 2.67 (m, 4H), 2.60 (t, 4H, J=4.6, 4.9 Hz), 2.52 (m, 2H), 1.01 (s, 6H). TLC: R_{f}=0.59 (1:9 MeOH:EtOAc). CHN: calculated for C₂₄H₂₈N₄O₂ 0.5 H₂O, C: 69.71%, H: 7.07%, N: 13.55%; found, C: 69.09%, H: 6.72%, N: 13.36%.

### Example 9

### PREPARATION OF 6-[2-(4-BENZO[D]ISOXAZOL-3-YL-PIPERAZIN-1-YL)-ETHYL]-3,4-DIMETHYL-1H-QUINOLIN-2-ONE

A. 6-(2-Chloroacetyl)-3,4-dimethyl-1H-quinolin-2-one

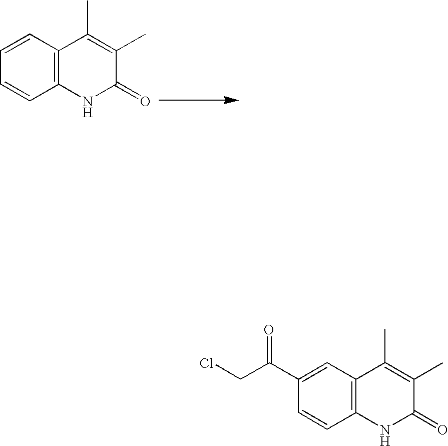

3,4-Dimethyl-1H-quinolin-2-one (Chem. Pharm. Bull., 1983, 31, 2986) underwent Friedel-Crafts acylation with chloroacetyl chloride according to the procedure described in step A of Example 1 to give the title compound as a solid. MS (APCI): (M+1)⁺=250.

B. 6-(2-Chloroethyl)-3,4-dimethyl-1H-quinolin-2-one

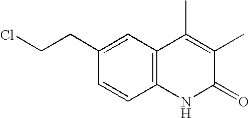

The compound described in step A above underwent treatment with triethylsilane in trifluoroacetic acid according to the procedure described in step B of Example 1 to give the title compound as a white, crystalline solid. MS (APCI): (M+1)⁺=236.

C. 6-[2-(4-Benzo[d]isoxazol-3-yl-piperazin-1-yl)-ethyl]-3,4-dimethyl-1H-quinolin-2-one hydrochloride

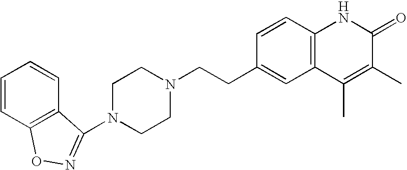

3-Piperazin-1-yl-benzo[d]isoxazole hydrochloride (1.0 g, 4.17 mmol) was reacted with the compound prepared in step B above (1.48 g, 6.26 mmol) based on the procedure given in step C of Example 1 to give the title compound which precipitated out of solution as an amorphous solid (730 mg). The solid was suspended in boiling MeOH and 4.0N HCl in dioxane was added until no further dissolution occurred. The remaining insolubles were filtered off and the filtrate was concentrated. The residue was washed with acetone to give the hydrochloride salt as a light beige powder, yield=707 mg (39%). MS (APCI): (M+1)⁺=403; (M−1)⁺=401. ¹H-NMR (DMSO-d₆, δ): 11.64 (s, 1H), 10.94 (br s, 1H), 8.02 (d, 1H, J=8.1 Hz), 7.61 (m, 3H), 7.33 (m, 2H), 7.22 (d, 1H, J=8.3 Hz), 4.13 (br d, 2H, J=13.4 Hz), 3.64 (brd, 2H, J=12.2 Hz), 3.50 (br t, 2H, J=11, 12 Hz), 3.41 (m, 2H), 3.29 (m, 2H), 3.13 (m, 2H), 2.39 (s, 3H), 2.08 (s, 3H).

### Example 10

### 6-[2-(4-BENZO[D]ISOXAZOL-3-YL-PIPERAZIN-1-YL)-ETHYL]-3,4-DIMETHYL-3,4-DIHYDRO-1H-QUINOLIN-2-ONE

A. 6-(2-Chloroacetyl)-3,4-dimethyl-3,4-dihydro-1H-quinolin-2-one

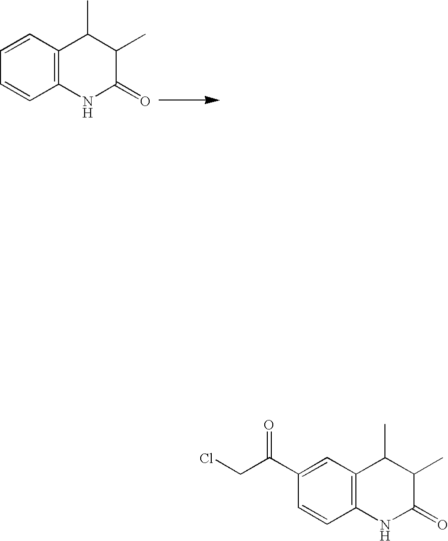

3,4-Dimethyl-3,4-dihydro-1H-quinolin-2-one (J. Chem. Soc. Perkin 1, 1981, 2912) underwent Friedel-Crafts acylation with chloroacetyl chloride based on the procedure described in step A of Example 1 to give the title compound as a solid. MS (APCI): (M+1)⁺=252.

B. 6-(2-Chloroethyl)-3,4-dimethyl-3,4-dihydro-1H-quinolin-2-one

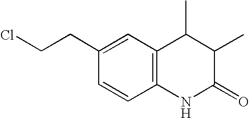

The compound prepared in step A above underwent reduction with triethylsilane in trifluoroacetic acid according to the procedure given in step B of Example 1 to give the title compound as a solid. MS (APCI): (M+1)⁺=238.

C. 6-[2-(4-Benzo[d]isoxazol-3-yl-piperazin-1-yl)-ethyl]-3,4-dimethyl-3,4-dihydro-1H-quinolin-2-one

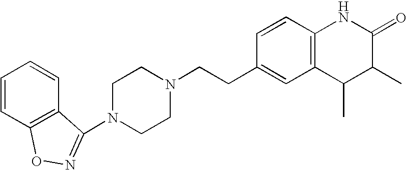

3-Piperazin-1-yl-benzo[d]isoxazole hydrochloride (1.50 g, 6.26 mmol) underwent reaction with the compound prepared in step B above (2.23 g, 9.39 mmol) according to the procedure given in step C of Example 1 to give the title compound as an off-white amorphous solid after elution through a flash column (silica gel 60, 230-400 mesh, 1:4 hexanes:EtOAc), yield=1.35 g (53%). MS (APCI): (M+1)⁺=405; (M−1)⁺=403. ¹H-NMR (DMSO-d₆, δ): 9.95 (s, 1H), 7.95 (d, 1H, J=8.1 Hz), 7.54 (d, 2H, J=3.9 Hz), 7.26 (m, 1H), 7.04 (s, 1H), 6.98 (m, 1H), 6.73 (d, 1H, J=7.8 Hz), 3.45 (t, 4H, J=4.6, 4.9 Hz), 2.67 (m, 3H), 2.60 (t, 4H, J=4.9, 4.9 Hz) 2.48 (m, 2H), 2.22 (m, 1H), 1.10 (d, 3H, J=7.1 Hz), 0.99 (q, 3H, J=6.8, 7.1, 8.8 Hz). TLC: R_{f}=0.28 (EtOAc). CHN: calculated for C₂₄H₂₈N₄O₂, C: 71.26%, H: 6.98%, N: 13.85%; found, C: 71.11%, H: 7.04%, N: 13.75%.

### Example 11

### 6-[2-(4-BENZO[D]ISOXAZOL-3-YL-PIPERAZIN-1-YL)-ETHYL]-1,3,3,4,4-PENTAMETHYL-3,4-DIHYDRO-1H-QUINOLIN-2-ONE

A. 6-(2-Chloroacetyl)-1,3,3,4,4-pentamethyl-3,4-dihydro-1H-quinolin-2-one

1,3,3,4,4-Pentamethyl-3,4-dihydro-1H-quinolin-2-one (4.21 g, 0.0193 mol, J. Chem. Soc., (C), 1971, 3769) underwent Friedel-Crafts acylation with chloroacetyl chloride (2.78 ml, 0.0348 mol) according to the procedure described in step A of Example 1 to give the title compound as an oil which gradually solidified upon stirring in aqueous solution, yield=5.65 g (99%). MS (APCI): (M+1)⁺=294; (M−1)⁺=292; (M+3)⁺=296.

B. 6-(2-Chloroethyl)-1,3,3,4,4-pentamethyl-3,4-dihydro-1H-quinolin-2-one

The reduction of the ketone in step A above (5.65 g, 0.0192 mol) was done according to the procedure given in step B of Example 1 to give, after elution through a flash column (silica gel 60, 230-400 mesh, 4:1 hexanes:EtOAc), an oil which crystallized on standing. Yield=4.71 g (88%). MS (APCI): (M+1)⁺=280; (M+3)⁺=282.

C. 6-[2-(4-Benzo[d]isoxazol-3-yl-piperazin-1-yl)-ethyl]-1,3,3,4,4-pentamethyl-3,4-dihydro-1H-quinolin-2-one

3-Piperazin-1-yl-benzo[d]isoxazole hydrochloride (1.0 g, 4.17 mmol) was reacted with the compound prepared in step B above (1.15 g, 4.11 mmol) based on the procedure in step C of Example 1 to give 773 mg (42%) of the title compound which precipitated out of solution as a white, amorphous solid. MS (APCI): (M+1)⁺=447. ¹H-NMR (DMSO-d₆, δ): 7.96 (d, 1H, J=8.1 Hz), 7.54 (d, 2H, J=3.9 Hz), 7.26 (m, 1H), 7.16 (s, 1H), 7.11 (d, 1H, J=8.3 Hz), 6.96 (d, 1H, J=8.1 Hz), 3.45 (br s, 4H), 3.24 (s, 3H), 2.72 (m, 2H), 2.62 (br s, 4H), 2.55 (t, 2H, J=8.3, 6.3 Hz), 1.05 (m, 12H). TLC: R_{f}=0.53 (EtOAc). CHN: calculated for C₂₇H₃₄N₄O₂ 0.5 H₂O, C: 71.18%, H: 7.74%, N: 12.30%; found, C: 70.74%, H: 7.46%, N: 12.16%.

### Example 12

### 6-[2-(4-BENZO[D]ISOXAZOL-3-YL- PIPERAZIN-1-YL)-ETHYL]-3,3,4-TRIMETHYL-3,4-DIHYDRO-1H-QUINOLIN-2-ONE

A. 6-(2-Chloroacetyl)-3,3,4-trimethyl-3,4-dihydro-1H-quinolin-2-one 3,3,4-Trimethyl-3,4-dihydro-1H-quinolin-2-one (5.0 g, 0.0264 mol, J. Am. Chem. Soc., 1956, 78, 2242) underwent Friedel-Crafts acylation with chloroacetyl chloride (3.79 ml, 0.0475 mol) according to the procedure described in step A of Example 1 to give the title compound as an amorphous, yellow solid, yield=7.02 g (100%). MS (APCI): (M+1)⁺=266; (M−1)⁺=264; (M+3)⁺=268.

B. 6-(2-Chloroethyl)-3,3,4-trimethyl-3,4-dihydro-1H-quinolin-2-one

The ketone of the compound in step A above (7.02 g, 0.0264 mol) was reduced according to the procedure in example 2 to give the title compound as a yellow, amorphous solid, yield=5.12 g (77%). MS (APCI): (M+1)⁺=252; (M−1)⁺=250; (M+3)⁺=254.

C. 6-[2-(4-Benzo[d]isoxazol-3-yl-piperazin-1-yl)-ethyl]-3,3,4-trimethyl-3,4-dihydro-1H-quinolin-2-one

3-Piperazin-1-yl-benzo[d]isoxazole hydrochloride (1.0 g, 4.16 mmol) was reacted with the compound prepared in step B above (1.57 g, 6.24 mmol) based on the procedure given in step C of Example 1 to give the title compound, which was eluted through a flash column (silica gel 60, 230-400 mesh, 4:1 EtOAc: hexanes) and further washed with acetone to give a white, crystalline solid. Yield=803 mg (46%). MS (APCI): (M+1)⁺=419; (M−1)⁺=417. ¹H-NMR (DMSO-d₆, δ): 9.91 (s, 1H), 7.95 (d, 1H, J=8.3 Hz), 7.54 (d, 2H, J=3.9 Hz), 7.25 (m, 1H), 7.00 (s, 1H), 6.97 (d, 1H, J=8.1 Hz), 6.71 (d, 1H, J=8.1 Hz), 3.45 (t, 4H, J=4.6, 5.1 Hz), 2.67 (m, 3H), 2.60 (t, 4H, J=4.4, 4.9 Hz), 2.52 (m, 2H), 1.01 (d, 3H, J=7.1 Hz), 0.98 (d, 6H, J=8.5 Hz). TLC: R_{f}=0.41 (EtOAc). CHN: calculated for C₂₅H₃₀N₄O₂, C: 71.74%, H: 7.22%, N: 13.39%; found, C: 71.71%, H: 7.28%, N: 13.24%.

### Example 13

### 6-{2-[4-(1H-INDAZOL-3-YL)-PIPERAZIN-1-YL]-ETHYL}-4-METHYL-3,4-DIHYDRO-1H-QUINOLIN-2-ONE

A. 3-Piperazin-1-yl-1H-indazole hydrochloride

A mixture of 3-chloro-1H-indazole (15.72 g, 0.103 mol, Aldrich) and piperazine (46 g, 0.534 mol, Aldrich) was heated at 250° C. in a sealed, stainless steel vessel for 14 hours. The viscous residue was partitioned between 1.0 N aqueous sodium hydroxide (NaOH) solution and methylene chloride and the organic layer was isolated, dried over magnesium sulfate, and filtered. The filtrate was treated with 4.0N hydrochloric acid (HCl) in dioxane, precipitating a greenish gum. The solvent was decanted and the gummy residue was taken up in water whereupon a small amount of disubstituted indazolyl piperazine precipitated (1.45 g, MS (APCI): (M+1)⁺=319). The precipitate was filtered off and the filtrate was concentrated to give a green, amorphous solid, yield=19.03 g (77%). MS (APCI): (M+1)⁺=203; (M−1)⁺=201.

B. 6-{2-[4-(1H-Indazol-3-yl)-piperazin-1-yl]-ethyl}-4-methyl-3,4-dihydro-1H-quinolin-2-one

3-Piperazin-1-yl-1H-indazole hydrochloride (2.0 g, 9.9 mmol) was reacted with the compound prepared in step B of Example 1 (2.22 g, 9.9 mmol) based on the procedure described in step C of Example 1 to give the title compound which was purified by elution through a flash column (silica gel 60, 230-400 mesh, 5% methanol (MeOH) in ethyl acetate (EtOAc) to 10% MeOH in EtOAc) and further washed with MeOH to give an off-white, amorphous solid. Yield=685 mg (18%). MS (APCI): (M+1)⁺⁼390; (M+1)⁺=388. ¹H-NMR (DMSO-d₆, δ): 11.94 (s, 1H), 9.98 (s, 1H), 7.70 (d, 1H, J=8.3 Hz), 7.31 (d,1H, J=8.3 Hz), 7.24 (t,1H, J=6.6, 7.8 Hz), 7.04 (s, 1H), 6.99 (d, 1H, J=8.1 Hz), 6.93 (t, 1H, J=7.1, 7.1 Hz), 6.73 (d, 1H, J=7.8 Hz), 3.29 (br s, 4H), 2.98 (q, 1H, J=6.6, 6.6, 6.6 Hz), 2.68 (br t, 2H, J=6.6, 8.5 Hz), 2.61 (br s, 4H), 2.51 (m, 2H), 2.17 (dd, 1H, J=7.1, 7.1 Hz), 1.14 (d, 3H, J=6.8 Hz). TLC: R_{f}=0.16 (1:9 MeOH:EtOAc, fluorescent). CHN: calculated for C₂₃H₂₇N₅O 0.25 C₄H₈O₂, C: 70.05%, H: 7.10%, N: 17.02%; found, C: 69.54%, H: 6.90%, N: 17.32%.

The alkylation procedure described in step C of Example 1 was applied as a general procedure for the synthesis of the following indazole analogs:

### Example 14

### 6-{2-[4-(1H-INDAZOL-3-YL)-PIPERAZIN-1-YL]-ETHYL}-4,4-DIMETHYL-3,4-DIHYDRO-1H-QUINOLIN-2-ONE

The title compound was prepared from 3-piperazin-1-yl-1H-indazole hydrochloride (382 mg, 1.60 mmol) and the compound prepared in step D of Example 5 (571 mg, 2.40 mmol). The product obtained was purified by elution through a flash column (silica gel 60, 230-400 mesh, 8% MeOH in EtOAc) to give an off-white, foamy solid, yield=221 mg (34%). MS (APCI): (M+1)⁺=404; (M−1)⁺=402. ¹H-NMR (DMSO-d₆, δ): 11.94 (s, 1H), 10.02 (s, 1H), 7.70 (d, 1H, J=8.3 Hz), 7.31 (d, 1H, J=8.3 Hz), 7.24 (t, 1H, J=6.8, 8.3 Hz), 7.14 (s, 1H), 6.98 (d, 1H, J=6.4 Hz), 6.93 (t, 1H, J=7.8, 7.1 Hz), 6.74 (d, 1H, J=8.1 Hz), 3.28 (br s, 4H), 2.68 (br t, 2H, J=6.3, 8.5 Hz), 2.61 (br s, 4H), 2.51 (br t, 2H, J=8.5, 7.1 Hz), 2.28 (s, 2H), 1.18 (s, 6H). TLC: R_{f}=0.25 (1:9 MeOH:EtOAc, fluorescent). CHN: calculated for C₂₄H₂₉N₅O 0.2 C₄H₈O₂, C: 70.73%, H: 7.32%, N: 16.63%; found, C: 70.22%, H: 7.19%, N: 16.45%.

### Example 15

### 6-{2-[4-(1H-INDAZOL-3-YL)-PIPERAZIN-1-YL]-ETHYL}-1,4,4-TRIMETHYL-3,4-DIHYDRO-1H-QUINOLIN-2-ONE

The title compound was prepared from the compound prepared in step A of Example 13 (700 mg, 2.93 mmol) and the compound prepared in step C of Example 6 (1.11 g, 4.40 mmol). The crude product was eluted through a flash column (silica gel 60, 230-400 mesh, 3% MeOH in EtOAc to 5% MeOH in EtOAc) to give an oil which crystallized on standing, yield=430 mg (35%). MS (APCI): (M+1)⁺=418; (M−1)⁺=416. ¹H-NMR (DMSO-d₆, δ): 11.94 (s, 1H), 7.70 (d, 1H, J=8.1 Hz), 7.31 (d, 1H, J=8.3 Hz), 7.24 (t, 1H, J=6.8, 8.3 Hz), 7.19 (s, 1H), 7.12 (d, 1H, J=8.1 Hz), 6.99 (d, 1H, J=8.3 Hz), 6.93 (t, 1H, J=7.3, 7.3 Hz), 3.30 (br s, 4H), 3.23 (s, 3H), 2.73 (t, 2H, J=7.3, 8.1 Hz), 2.62 (br s, 4H), 2.54 (t, 2H, J=8.1, 6.8 Hz), 2.38 (s, 2H), 1.18 (s, 6H). TLC: R_{f}=0.26 (1:9 MeOH:EtOAc, fluorescent). CHN: calculated for C₂₅H₃₁N₅O, C: 71.91%, H: 7.48%, N: 16.77%; found, C: 71.49%, H: 7.57%, N: 16.47%.

### Example 16

### 6-{2-[4-(1H-INDAZOL-3-YL)-PIPERAZIN-1-YL]-ETHYL}-3-METHYL-3,4-DIHYDRO-1H-QUINOLIN-2-ONE

The title compound was prepared from the compound prepared in step A of Example 13 (2.0 g, 9.9 mmol) and the compound prepared in step B of Example 7 (2.21 g, 9.9 mmol). The product was purified by elution through a flash column (silica gel 60, 230-400 mesh, 5% MeOH in EtOAc to 10% MeOH in EtOAc) followed by. washing with acetone to give a white, amorphous solid, yield=670 mg (17%). MS (APCI): (M+1)⁺=390; (M−1)⁺=388. ¹H-NMR (DMSO-d₆, δ): 11.94 (s, 1H), 9.94 (s, 1H), 7.70 (d, 1H, J=8.3 Hz), 7.31 (d, 1H, J=8.3 Hz), 7.24 (t, 1H, J=7.1, 8.1 Hz), 7.01 (s, 1H), 6.97 (d, 1H, J=8.1 Hz), 6.93 (t, 1H, J=7.8, 7.1 Hz), 6.72 (d, 1H, J=8.1 Hz), 3.30 (br s, 4H), 2.85 (dd, 1H, J=5.9, 5.6 Hz), 2.66 (t, 2H, J=7.3, 8.5 Hz), 2.61 (br s, 4H), 2.51 (m, 4H), 1.07 (d, 3H, J=6.8 Hz). TLC: R_{f}=0.24 (1:9 MeOH:EtOAc, fluorescent). CHN: calculated for C₂₃H₂₇N₅O, C: 70.93%, H: 6.99%, N: 17.98%; found, C: 70.58%, H: 6.74%, N: 17.81%.

### Example 17

### 6-{2-[4-(1H-INDAZOL-3-YL)-PIPERAZIN-1-YL]-ETHYL}-3,3-DIMETHYL-3,4-DIHYDRO-1H-QUINOLIN-2-ONE

The title compound was prepared from the compound prepared in step A of Example 13 (2.0 g, 9.9 mmol) and the compound prepared in step B od Example 8 (2.35 g, 9.9 mmol). The product was purified by elution through a flash column (silica gel 60, 230-400 mesh, 5% MeOH in EtOAc to 10% MeOH in EtOAc), followed by washing with acetone to give a white, amorphous solid, yield=675 mg (17%). MS (APCI): (M+1)⁺=404; (M−1)⁺=402. ¹H-NMR (DMSO-d₆, δ): 11.94 (s, 1H), 9.91 (s, 1H), 7.70 (d, 1H, J=8.1 Hz), 7.31 (d, 1H, J=8.3 Hz), 7.24 (t, 1H, J=7.1, 8.1 Hz), 6.98 (d, 2H, J=8.3 Hz), 6.93 (t, 1H, J=7.8, 7.1 Hz), 6.73 (d, 1H, J=7.8 Hz), 3.28 (br s, 4H), 2.66 (m, 4H), 2.61 (br s, 4H), 2.51 (br t, 2H, J=8.5, 6.8 Hz), 1.00 (s, 6H). TLC: R_{f}=0.22 (1:9 MeOH:EtOAc, fluorescent). CHN: calculated for C₂₄H₂₉N₅O, C: 71.44%, H: 7.24%, N: 17.36%; found, C: 71.24%, H: 7.16%, N: 17.12%.

### Example 18

### 6-{2-[4-(1H-INDAZOL-3-YL)-PIPERAZIN-1-YL]-ETHYL}-3,4-DIMETHYL-3,4-DIHYDRO-1H-QUINOLIN-2-ONE

The title compound was prepared from the compound prepared in step A of Example 13 (1.0 g, 4.19 mmol) and the compound prepared in step B of Example 10 (1.50 g, 6.29 mmol). The crude product was eluted through a flash column (silica gel 60, 230-400 mesh, 3% MeOH in EtOAc to 5% MeOH in EtOAc) to give the title compound as a white, foamy solid, yield=781 mg (46%). MS (APCI): (M+1)⁺=404; (M−1)⁺=402. ¹H-NMR (DMSO-d₆, δ): 11.94 (s, 1H), 9.96 (d, 1H, J=13.2 Hz), 7.70 (d, 1H, J=8.1 Hz), 7.31 (d, 1H, J=8.3 Hz), 7.23 (t, 1H, J=6.8, 8.3 Hz), 7.01 (m, 2H), 6.93 (t, 1H, J=7.8, 7.1 Hz), 6.72 (t, 1H, J=7.8, 7.1 Hz), 3.28 (br s, 4H), 2.91 (m, 1H), 2.67 (m, 2H), 2.61 (br s, 4H), 2.54 (m, 2H), 2.22 (m, 1H), 1.10 (d, 2H, J=7.1 Hz), 0.98 (q, 4H, J=6.3, 7.1, 8.8 Hz). TLC: R_{f}=0.24 (1:9 MeOH:EtOAc, fluorescent). CHN: calculated for C₂₄H₂₉N₅O 0.6 H₂O, C: 69.57%, H: 7.35%, N: 16.90%; found, C: 69.22%, H: 6.92%, N: 16.58%.

The 1,2-benzisothiazole analogs of Examples 19 and 20 were prepared using the procedure described in Step C of Example 1.

### Example 19

### 6-[2-(4-BENZO[D]ISOTHIAZOL-3-YL-PIPERAZIN-1-YL)-ETHYL]-1,3,3,4,4-PENTAMETHYL-3,4-DIHYDRO-1H-QUINOLIN-2-ONE HYDROCHLORIDE

From 3-Piperazin-1-yl-benzo[d]isothiazole hydrochloride (1.0 g, 3.91 mmol, J. Med. Chem., 1986, 29, 359) and compound produced in step B of Example 11 (1.64 g, 5.86 mmol). The crude product was eluted through a flash column (silica gel 60, 230-400 mesh, 3:7 hexanes:EtOAc) to give a clear oil. The oil was dissolved in methylene chloride and the solution treated with 4.0N HCl in dioxane to precipitate the hydrochloride salt as a white, amorphous solid, yield=1.05 g (54%). MS (APCI): (M+1)⁺=463. ¹H-NMR (DMSO-d₆, δ): 11.20 (br s, 1H), 8.11 (d, 1H, J=8.1 Hz), 8.08 (d, 1H, J=8.3 Hz), 7.57 (t, 1H, J=7.1, 7.1 Hz), 7.44 (t, 1H, J=7.3, 7.1 Hz), 7.21 (s, 1H), 7.16 (d, 1H, J=8.1 Hz), 7.04 (d, 1H, J=8.3 Hz), 4.07 (br d, 2H, J=13.4 Hz), 3.65 (br d, 2H, J=11.5 Hz), 3.50 (br t, 2H, J=12.2, 11.9 Hz), 3.37 (m, 4H), 3.32 (s, 3H), 3.06 (m, 2H), 1.07 (br s, 12H). TLC: R_{f}=0.49 (EtOAc). CHN: calculated for C₂₇H₃₄N₄OS 1.1 HCl, C: 64.50%, H: 7.04%, N: 11.14%; found, C: 64.05%, H: 7.07%, N: 11.00%.

### Example 20

### 6-[2-(4-BENZO[D]ISOTHIAZOL-3-YL-PIPERAZIN-1-YL)-ETHYL]-3,3,4-TRIMETHYL-3,4-DIHYDRO-1H-QUINOLIN-2-ONE

The title compound was prepared from 3-piperazin-1-yl-benzo[d]isothiazole hydrochloride (1.0 g, 3.91 mmol) and the compound prepared in step B of Example 12 (1.48 g, 5.86 mmol). The title compound precipitated out of solution as a white, amorphous solid, yield=1.22 g (72%). MS(APCI): (M+1)⁺=435; (M−1)⁺=433. ¹H-NMR (DMSO-d₆, δ): 9.91 (s, 1H), 8.02 (d, 2H, J=8.3 Hz), 7.52 (t, 1H, J=7.3, 7.1 Hz), 7.40 (t, 1H, J=7.1, 7.3 Hz), 7.00 (s, 1H), 6.97 (d, 1H, J=8.1 Hz), 6.71 (d, 1H, J=8.1 Hz), 3.41 (br s, 4H), 2.60 (m, 9H), 1.01 (d, 3H, J=7.1 Hz), 0.98 (d, 6H, J=8.3 Hz). CHN: calculated for C₂₅H₃₀N₄OS 0.8 H₂O, C: 66.87%, H: 7.09%, N: 12.48%; found, C: 66.34%, H: 6.75%, N: 12.28%.

### Example 21

### 6-[2-(4-BENZO[D]ISOTHIAZOL-3-YL-PIPERAZIN-1-YL)-ETHYL]-4,4,8-TRIMETHYL-3,4-DIHYD RO-1H-QUINOLIN-2-ONE

A. 3-Methyl-but-2-enoic-acid o-tolylamide

To a cold 0.25M solution of o-toluidine (5.0 ml, 46.85 mmole, 1 eq) in dry THF and pyridine (2 eq) was added dropwise neat 3,3-dimethyl-acryloyl chloride and stirred vigorously. The reaction was filtered and the filtrate diluted with EtOAc (equal volume) and washed with H₂O (3×), 1N HCl (2×), saturated sodium carbonate (Na₂CO₃) (1×), brine (1×), dried (MgSO₄), and concentrated to a solid. A mixture of the titled product and its terminal olefin isomer were isolated as a 1:1 mixture. MS (APCI)=190.1 [M+H]⁺.

B. 4,4,8-Trimethyl-3,4-dihydro-1H-quinolin-2-one

To a solution of 3-Methyl-but-2-enoic-acid o-tolylamide (7.27 g, 38.41 mmole, 1 eq) in 1 ,2-dichlorobenzene (50 ml) was added aluminum chloride (AlCl₃) (30.73 g, 230.49 mmole, 6 eq) and the whole heated to 50-70° C. As the reaction reached about 50° C. vigorous HCl(g) was released. After the HCl evolution appeared to cease, the reaction was allowed to continue for an additional 10 min before cooling. The reaction was cooled and poured into cold H₂O. The heterogeneous mix was extracted with CH₂Cl₂ (3×100 ml), dried (MgSO₄) and concentrated to an orange oil which was purified by MPLC (30% EA/Hex) to give the above titled compound (5.357 g, 28.31 mmole, 74% yield). ¹H NMR (400 MHz, CDCl₃) δ 8.43 (s, 1H), 7.16 (d, J=7.5 Hz, 1H), 7.04 (d, J=7.5 Hz, 1H), 6.96 (t, J=7.5 Hz, 1H), 2.48 (s, 2H), 2.30 (s, 3H), 1.32 (s, 6H).

C. 6-(2-Chloro-acetyl)-4,4,8-trimethyl-3,4-dihydro-1H-quinolin-2-one

To a solution of 4,4,8-Trimethyl-3,4-dihydro-1H-quinolin-2-one (3.545 g, 18.71 mmole, 1 eq) in CS₂ (200 ml) was added chloroacetyl choride (2.23 ml, 28.06 mmole, 1.5 eq), followed by aluminum chloride (9.98 g, 74.84 mmole, 4 eq) in one portion. The reaction was heated to reflux for 1 h after which thin layer chromatography (TLC) and MS indicated complete reaction. After cooling, the solvent was decanted and the remaining residue was carefully hydrolyzed with cold H₂O. The resulting precipitate was filtered and dried at 50° C. under hivac to give titled compound as a tan solid (4.79 g, 18.03 mmole, 96% yield). 100% purity at 254 nm; LCMS (APCI) 266.3 [M+H]⁺; ¹H NMR (400 MHz, CDCl₃) δ 7.89 (bs, 1H), 7.81 (s, 1H), 7.67 (s, 1H), 4.65 (s, 2H), 2.52 (s, 2H), 2.32 (s, 3H), 1.36 (s, 6H).

D. 6-(2-Chloro-ethyl)-4,4,8-trimethyl-3,4-dihydro-1H-quinolin-2-one

To a solution of 6-(chloromethylcarbonyl)-4,4,8-trimethyl-3,4-dihydro-1H-quinolin-2-one (4.79 g, 18.03 mmole, 1.0 eq) in trifluoroacetic acid (100 ml) was added triethylsilane (7.20 ml, 45.08 mmole, 2.5 eq) and the whole heated to 60° C. After 2 hours TLC (30%EtOAc/Hexanes (Hex)) and MS indicated complete reaction. The reaction was cooled and poured over ice. After extracting with CH₂Cl₂ (3×100 ml), drying (MgSO₄) and concentrating to an oil, the crude was purified by MPLC (30% EtOAc/Hex) to give the title compound as a white solid (3.23 g, 12.84 mmole, 71% yield). 100% purity at 254 nm; LCMS (APCI) 252.2 [M+H]⁺; ¹H NMR (400 MHz, CDCl₃) δ 7.41 (bs, 1H), 6.99 (s, 1H), 6.89 (s, 1H), 3.67 (t, J=7.3 Hz, 2H), 2.98 (t, J=7.3 Hz, 2H), 2.46 (s, 2H), 2.21 (s, 3H), 1.30 (s, 6H).

E. 6-[2-(4-Benzo[d]isothiazol-3-yl-piperazin-1-yl)-ethyl]-4,4,8-trimethyl-3,4-dihydro-1H-quinolin-2-one

A heterogeneous mix of 6-(chloromethylcarbonyl)-4,4,8-trimethyl-3,4-dihydro-1H-quinolin-2-one (2.200 g, 8.739 mmole, 1.0 eq), sodium carbonate (1.158 g, 10.924 mmole, 1.25 eq), sodium iodide (0.131 g, 0.874 mmole, cat.), and added 3-piperazin-1-yl-benzo[d]isothiazole hydrochloride (3.353 g, 13.110 mmole, 1.5 eq) in acetonitrile (35 ml) was heated to 150° C. under microwave assistance for 30 min. The reaction was diluted with H₂O (100 ml), CH₂Cl₂ (100 ml) and the layers separated. The aqueous layer was extracted with CH₂Cl₂ (2×, 50 ml) and the organic layer dried over magnesium sulfate (MgSO₄), concentrated, and the residue purified by MPLC (25%EA/CH₂Cl₂ - - - 50% EA gradient over 20 min and hold for 20 min - - - 100% EA gradient over 20 min). The title compound was obtained as a white crystalline solid in 63% yield with 30% recovered starting material (6-(2-chloro-ethyl)-4,4,8-trimethyl-3,4-dihydro-1H-quinolin-2-one). ¹H NMR (400 MHz, CDCl₃) δ 7.90 (d, 1H, J=7.94 Hz), 7.80 (d, 1H, J=7.94 Hz), 7.46 (t, 1H, J=7.94 Hz), 7.34 (t, 1H, J=7.94 Hz), 7.02 (s, 1H) 6.91 (s, 1H), 4.78 (s, 1H), 3.69-3.55 (m, 4H), 2.86-2.59 (m, 8H), 2.45 (s, 2H), 2.21 (s, 3H), 1.30 (s, 6H).

F. 6-[2-(4-Benzo[d]isothiazol-3-yl-piperazin-1-yl)-ethyl]-4,4,8-trimethyl-3,4-dihydro-1H-quinolin-2-one mesylate salt

The free base (319.77 g, 0.735 mol) was dissolved in tetrahydrofuran (3.0 liters) and the solution was heated to 60° C. Methanesulfonic acid (74.25 g, 0.773 mol) was added over a period of 5 minutes (CAUTION: Exothermic) and the reaction mixture was vigorously stirred until it cooled to room temperature. The precipitate was collected and recrystallized from water (6.0 liters). Yield=333 grams (85%), ¹H-NMR (CDCl₃, δ): 11.69 (br s, 1H), 7.84 (cm, 2H), 7.52 (cm, 1H), 7.48 (br s, 1H), 7.41 (cm, 1H), 7.06 (br s, 1H), 6.96 (br s, 1H), 4.16 (m, 2H), 4.00 (m, 2H), 3.64 (m, 2H), 3.13-3.28 (cm, 6H), 2.91 (s, 3H), 2.45 (s, 2H), 2.21 (s, 3H), 1.30 (s, 6H). CHN: Calculated for C₂₆H₃₄N₄O₄S₂: C, 58.84%; H, 6.46%; N, 10.56%; S, 12.08%; found, C, 58.83%; H, 6.29%; N, 10.44%; S, 12.37%.

### Example 22

### 2-[2-(4-BENZO[D]ISOTHIAZOL-3-YL-PIPERAZIN-1-YL)-ETHYL]-4,4,8-TRIMETHYL-3,4-DIHYDRO-1H-QUINOLIN-2-ONE

To a solution of 6-(2-chloro-ethyl)-4,4,8-trimethyl-3,4-dihydro-1H-quinolin-2-one (1.5 eq) in dioxane/H₂O (0.03M 1:1) was added sodium carbonate (2.2 eq), sodium iodide (catalytic), and added 3-piperazin-1-yl-benzo[d]isothiazole hydrochloride (1.0 eq). The reaction mixture was heated to reflux for 24-72 hours. The reaction mixture was then concentrated and partitioned between H₂O and CH₂Cl₂. The organic layer was dried (MgSO₄), concentrated and purified by chromatography (4:1 EA/Hex) to yield the title compound in 15-48% yield. LC/MS column: Phenomenex Develosil Combi-RP-3 3μ50×4.6 mm, length 150×4.6

### Example 23

### 6-[2-(4-BENZO[D]ISOTHIAZOL-3-YL-PIPERAZIN-1-YL)-ETHYL]-7-CHLORO-4,4,8-TRIMETHYL-3,4-DIHYDRO-1H-QUINOLIN-2-ONE METHANESULFONATE

A. 3-Methyl-but-2-enoic Acid (3-chloro-2-methyl-phenyl)-amide

3,3-Diethylacryoyl chloride (21.0 mL, 0.189 mol) was slowly added to a solution of 3-chloro-2-methylaniline (20.0 mL, 0.167 mol) and pyridine (17.0 mL, 0.210 mol) in dichloromethane (210 mL) at 0° C. After 1.5 h, the reaction was quenched by slow addition of saturated sodium bicarbonate solution (60 mL). The solution was transferred to a 500 mL separatory funnel and the layers separated. The aqueous layer was back-extracted with dichloromethane (2×100 mL). The combined organic extracts were dried over anhydrous sodium sulfate, filtered and the solvent removed under reduced pressure. The resulting purple solid was used directly without purification. MS (APCI): (M+1)⁺=224.1.

B. 7-Chloro-6-(2-chloro-acetyl)-4,4,8-trimethyl-3,4-dihydro-1H-quinolin-2-one

The compound prepared in step A above was dissolved in dichloromethane (167 mL). Aluminum chloride (91.5 g, 0.686 mol) was slowly added to the reaction mixture at a rate to maintain gentle reflux. Upon complete addition of the aluminum chloride, a reflux condenser was attached and the reaction was heated to reflux. After 1.5 h, TLC showed no remaining starting material. Chloroacetyl chloride (20.0 mL, 0.250 mol) was slowly added and the mixture was refluxed for an additional 4 h. The reaction mixture was poured into ice water (1000 mL) and extracted with dichloromethane (4×300 mL). The organic extracts were combined, washed with saturated sodium chloride solution (200 mL), dried over anhydrous sodium sulfate, filtered and the solvent removed under reduced pressure. The resulting solid was used directly without purification. MS (APCI): (M+1)⁺=300.1, (M+3)⁺=302.1.

C. 7-Chloro-6-(2-chloro-ethyl)-4,4,8-trimethyl-3,4-dihydro-1H-quinolin-2-one

The compound prepared in step B above was dissolved in trifluoroacetic acid (168.0 mL). Triethylsilane (59.0 mL, 0.369 mol) was added to the solution and the reaction mixture heated to 60° C. under nitrogen. After 5.5 h, the reaction was cooled to room temperature and the reaction was stirred overnight. The reaction mixture was poured into ice water (350 mL). The reaction flask was rinsed with methanol (50 mL). The mixture was vigorously stirred resulting in formation of a precipitate. The solid was filtered and then triturated with hexanes. The solid was recrystallized from hot methyl-tert-butyl ether (MTBE) (600 mL) to provide the titled compound (36.0345 g, 0.126 mol, 75% yield over four steps) as a light tan solid. MS (APCI): (M-1)⁺=286.1, (M+1)⁺=288.1. ¹H NMR (400 MHz, CDCl₃) δ 7.50 (br s, 1H), 7.06 (s, 1H), 3.71 (t, J=7.2 Hz, 2 H), 3.16 (t, J=7.2 Hz, 2 H), 2.45 (s, 2 H), 2.30 (s, 3 H), 1.30 (s, 6 H).

D. 6-[2-(4-Benzo[d]isothiazol-3-yl-piperazin-1-yl)-ethyl]-7-chloro-4,4,8-trimethyl-3,4-dihydro-1H-quinolin-2-one

A mixture of the product from step C above (5.0016 g, 17.476 mmol), 3-piperazin-1-yl-benzo[d]isothiazole hydrochloride (4.4811 g, 17.520 mmol), potassium carbonate (4.8299 g, 34.946 mmol) and potassium iodine (0.2903 g, 1.749 mmol) were reacted in acetonitrile (29.0 mL) in a CEM MARS5 microwave reactor for 1 h at 200° C. The reaction was cooled to room temperature, diluted with H₂O and filtered. The solid was washed with H₂O and hexanes. The resulting solid was purified by MPLC [silica gel, 100% methylene chloride (CH₂Cl₂) to 3% MeOH/CH₂Cl₂ over 1 h, then hold at 3% MeOH/CH₂Cl₂] to give 5.6591 g, (12.065 mmol, 69%) of the titled compound as an off-white crystalline solid. LC-MS (APCI): (M-1)⁺=469.1, (M+1)⁺=471.0.

E. 6-[2-(4-Benzo[d]isothiazol-3-yl-piperazin-1-yl)-ethyl]-7-chloro-4,4,8-trimethyl-3,4-dihydro-1H-quinolin-2-one Methanesulfonate

Methanesulfonic acid (0.139 mL, 2.142 mmol) was added to a hot solution of the product from step D above (1.0042 g, 2.141 mmol) in tetrahydrofuran (THF) (35.0 mL). The titled compound began crystallizing almost immediately. The reaction was slowly cooled to room temperature and after 2 h, 1.0813 g (1.913 mmol, 89%) of the titled compound was collected as a fine, white solid. No further purification was needed. ¹H NMR (400 MHz, CDCl₃) 61.31 (s, 6 H), 2.29 (s, 3 H), 2.44 (s, 2 H), 2.90 (s, 3H), 3.17-3.29 (m, 4H), 3.32-3.40 (m, 2H), 3.70 (d, J=11.3 Hz, 2H), 3.97 (t, J=12.1 Hz, 2H), 4.17 (d, J=14.4 Hz, 2H), 7.33 (s, 1H), 7.41 (t, J=8.0 Hz, 1H), 749-7.55 (m, 2H), 7.84 (t, J=7.8 Hz, 2H), 11.67 (br s, 1H). Anal. calculated (calcd.) for C₂₅H₂₉ClN₄OS.CH₄O₃S: C, 55.26; H, 5.89; N, 9.91. Found: 54.86; H, 5.83; N, 9.65.

### Example 24

### 6-[2-(4-BENZO[D]ISOTHIAZOL-3-YL-PIPERAZIN-1-YL)-ETHYL]-7-FLUORO-4,4,8-TRIMETHYL-3,4-DIHYDRO-1H-QUINOLIN-2-ONE HYDROCHLORIDE

A. 3-Methyl-but-2-enoic acid (3-fluoro-2-methyl-phenyl)-amide

The titled compound was prepared from 3-fluoro-2-methylaniline (2.30 mL, 20.197 mmol) and 3,3-dimethylacryloyl chloride (2.50 mL, 22.457) using the procedure described in step A of Example 23. The resulting semi-solid was used directly without purification. MS (APCI): (M+1)⁺=208.1.

B. 6-(2-Chloro-acetyl)-7-fluoro-4,4,8-trimethyl-3,4-dihydro-1H-quinolin-2-one

The titled compound was prepared from the compound in step A above, aluminum chloride (11.04 g, 82.796 mmol) and chloroacetyl chloride (2.40 mL, 30.005 mmol) using the procedure described in step B of Example 23. The product was crystallized from hot EtOAc/hexanes. The mother liquor was purified by MPLC (silica gel, 100% CH₂Cl₂ to 3% MeOH/CH₂Cl₂ over 1 h, then hold at 3% MeOH/CH₂Cl₂). The two batches were eqivalent by LC-MS and were combined to give 4.6617 g (16.430 mmol, 81% over three steps) of the titled compound as a white solid. MS (APCI): (M+1)⁺=284.2. ¹H NMR (400 MHz, CDCl₃) δ 7.79 (d, J=7.3 Hz 1H), 7.74 (br s, 1H), 4.69 (d, J=3.2 Hz, 2H), 2.50 (s, 2H), 2.20 (s, 3H), 1.34 (s, 6H).

C. 6-(2-Chloro-ethyl)-7-fluoro-4,4,8-trimethyl-3,4-dihydro-1H-quinolin-2-one

The titled compound was prepared from 6-(2-chloro-acetyl)-7-fluoro-4,4,8-trimethyl-3,4-dihydro-1H-quinolin-2-one (46.56 g, 0.164 mol), triethyl silane (55.0 mL, 0.344 mol) and trifluoroacetic acid (78.0 mL) using the procedure described in step C of Example 23. The reaction was quenched in ice water (400 mL) and the flask rinsed with MeOH (70 mL). A white solid formed. The solid was filtered and washed with hexanes. The solid was recrystallized from hot acetonitrile/MTBE to give 19.7280 g (73.137 mmol, 45%) of the titled compound as a white solid. MS (APCI): (M+1)⁺=270.1, (M+3)⁺=272.0. 1H NMR (400 MHz, CDCl₃) δ 1.29 (s, 6H), 2.14 (d, J=1.8 Hz, 3H), 2.45 (s, 2H), 3.04 (t, J=7.3 Hz, 2H), 3.68 (t, J=7.3 Hz, 2H), 6.97 (d, J=7.8 Hz, 1H), 7.68 (s,1H).

D. 6-[2-(4-Benzo[d]isothiazol-3-yl-Piperazin-1-yl)-ethyl]-7-fluoro-4,4,8-trimethyl-3,4-dihydro-1H-guinolin-2-one Hydrochloride

A mixture of 6-(2-chloro-ethyl)-7-fluoro-4,4,8-trimethyl-3,4-dihydro-1H-quinolin-2-one (0.7499 g, 2.780 mmol), 3-piperazin-1-yl-benzo[d]isothiazole hydrochloride (0.7834 g, 3.063 mmol), potassium carbonate (0.8456 g, 6.118 mmol) and potassium iodine (0.0495 g, 0.298 mmol) were reacted in acetonitrile (7.0 mL) in a CEM MARS5 microwave reactor for 1 h at 150° C. The reaction was cooled to room temperature, diluted with H₂O (70 mL) and extracted with dichloromethane (2×75 mL). The organic extracts were combined, dried over anhydrous sodium sulfate, filtered and the solvent removed under reduced pressure. The resulting solid was purified by MPLC (The solid was washed with H₂O and hexanes. The resulting solid was purified by MPLC (silica gel, 100% CH₂Cl₂ to 3% MeOH/CH₂Cl₂ over 1 h then hold at 3% MeOH/CH₂Cl₂) to give a mixture of the titled compound and the product of step C. This mixture was dissolved in dichloromethane and 4 M hydrogen chloride in dioxane was slowly added until the product precipitated. The titled compound (0.3137 g, 0.660 mmol, 53% over two steps) was isolated as a white solid. MS (APCI): (M+1, free base)⁺=439.2. ¹H NMR (400 MHz, CDCl₃) δ 1.29 (s, 6H), 2.12 (d, J=1.6 Hz, 3H), 2.44 (s, 2H), 3.19 (s, 4H), 3.32 (s, 2H), 3.59 (s, 2H), 4.17 (m, 4H), 7.12 (d, J=7.6 Hz, 1H), 7.38-7.45 (m, 2H), 7.49-7.54 (m, 1 H), 7.84 (t, J=8.8 Hz, 2H), 13.2 (br s, 1H). Anal. calcd. for C₂₄H₂₇FN₄OS.HCl.0.75 H₂O.0.10 CH₂Cl₂: 58.24; H, 6.02; N, 11.27; H₂O, 2.72. Found: 57.84; H, 6.17; N, 10.98; H₂O, 2.57.

### Example 25

### 6-[2-(4-BENZO[D]ISOTHIAZOL-3-YL-PIPERAZIN-1-YL)-ETHYL]-7-FLUORO-4,4,8-TRIMETHYL-3,4-DIHYDRO-1H-QUINOLIN-2-ONE METHANESULFONATE

A. 6-[2-(4-Benzo[d]isothiazol-3-yl-piperazin-1-yl)-ethyl]-7-fluoro-4,4,8-trimethyl-3,4-dihydro-1H-quinolin-2-one

A mixture of the product from step C of Example 24 (2.2896 g, 8.488 mmol), 3-piperazin-1-yl-benzo[d]isothiazole hydrochloride (2.4295 g, 8.489 mmol), potassium carbonate (2.3472 g, 16.983 mmol) and potassium iodine (0.1406 g, 0.847 mmol) were reacted in acetonitrile (14.0 mL) in a CEM MARS5 microwave reactor for 20 min at 175° C. The reaction was cooled to room temperature, diluted with H₂O and the resulting solid was filtered and washed with H₂O and hexanes. The solid was >98% pure by LC-MS. The while solid was dried in a vacuum over at 50° C. to give 3.2518 g (7.185 mmol, 85%) of the titled compound as a white solid. >98% pure by LC-MS. MS(APCl): (M+1)⁺=453.2.

B. 6-[2-(4-Benzo[d]isothiazol-3-yl-piperazin-1-yl)-ethyl1-7-fluoro-4,4,8-trimethyl-3,4-dihydro-1H-quinolin-2-one methanesulfonate

Methansulfonic acid (0.144 mL, 2.219 mmol) was added to a hot solution of the product of step A above (1.0054 g, 2.221 mmol) in THF (25.0 mL). The titled compound began crystallizing out immediately. The reaction mixture was slowly cooled to room temperature. After 3 h, the solid was filtered to give 1.1945 g (2.177 mmol, 98%) of the titled compound a fine, white solid. LC-MS (APCI): (M+1, free base)⁺=452.8. ¹H NMR (400 MHz, CDCl₃) δ 1.29 (s, 6H), 1.79-1.89 (m, 1H), 2.11 (d, J=1.4 Hz, 3H), 2.44 (s, 2H), 2.89 (s, 3H), 3.15-3.26 (m, 5H), 3.58-3.78 (m, 8H), 3.92-4.03 (m, 2H), 4.09-4.19 (m, 2H), 7.16 (d, J=8.0 Hz, 1H), 7.34 (s, 1H), 7.37-7.43 (m, 1H), 7.48-7.54 (m, 1H), 7.83 (d, J=7.6 Hz, 1H), 7.85 (d, J=7.6 Hz, 1H), 11.67 (br s, 1H). Anal. calc'd. for C₂₅H₂₉FN₄OS.CH₄O₃S: C, 56.91; H, 6.06; N, 11.66. Found: C, 56.60; H, 6.07; N, 9.91.

### Example 26

### 6-[2-(4-BENZO[D]ISOTHIAZOL-3-YL-PIPERAZIN-1-YL)-ETHYL]-8-ETHYL-4,4-DIMETHYL-3,4-DIHYDRO-1H-QUINOLIN-2-ONE

A. 3-Methyl-but-2-enoic acid (2-ethyl-phenyl)-amide

Prepared from 2-ethylaniline and 3,3-dimethylacryloyl chloride using the procedure described for Example 5A. Isolated in 100% purity @ 254 nm; LCMS (APCI): 204 [M+H]⁺.

B. 8-Ethyl-4,4-dimethyl-3,4-dihydro-1H-quinolin-2-one

Prepared from Example 26A using the methodology described for the preparation of Example 5B. Isolated in 100% purity @ 254 nm; LCMS (APCI): 204 [M+H]⁺.

C. 6-(2-Chloro-acetyl)-8-ethyl-4,4-dimethyl-3,4-dihydro-1H-quinolin-2-one

Prepared from the title compound of Example 26B using the methodology described for the preparation of Example 1A. Isolated in 100% purity @ 254 nm; LCMS (APCI): 280 [M+H]⁺.

D. 6-(2-Chloro-ethyl)-8-ethyl-4,4-dimethyl-3,4-dihydro-1H-quinolin-2-one

Prepared from the title compound of Example 26C using the methodology described for the preparation of Example 1B. Isolated in 100% purity @ 254 nm; LCMS (APCI): 266 [M+H]⁺.

E. 6-[2-(4-Benzo[d]isothiazol-3-yl-piperazin-1-yl)-ethyl]-8-ethyl-4,4-dimethyl-3,4-dihydro-1H-quinolin-2-one

Prepared from the title compound of Example 26D and 3-piperazin-1-yl-benzo[d]isothiazole hydrochloride according to the procedure described for the preparation of Example 25A. Isolated in 100% purity @ 254 nm; LCMS (APCI): 273 [M+H]⁺. 1H NMR (400 MHz, CHLOROFORM-D) δ ppm 1.23 (t, J=7.62 Hz, 3H) 1.31 (s, 6H) 2.46 (s, 2H) 2.53 (q, J=7.68 Hz, 2H) 2.61-2.72 (m, 2H) 2.72-2.88 (m, 6H) 3.60 (s, 4H) 6.93 (d, J=1.95 Hz, 1H) 7.03 (d, J=1.76 Hz, 1H) 7.32-7.39 (m, 2H) 7.45 (d, J=7.81 Hz, 1H) 7.81 (d, J=8.21 Hz, 1H) 7.90 (d, J=7.81 Hz, 1H). CHN: Calculated for C₂₆H₃₂N₄O₁S₁, C: 69.61%, H: 7.19%, N: 12.49%; found, C: 69.51%, H: 7.32%, N: 12.30%.

### Example 27

### 6-[2-(4-BENZO[D]ISOTHIAZOL-3-YL-PIPERAZIN-1-YL)-ETHYL]-8-CHLORO-4,4-DIMETHYL-3,4-DIHYDRO-1H-QUINOLIN-2-ONE HYDROCHLORIDE

A. 3-Methyl-but-2-enoic acid (2-chloro-phenyl)-amide

Prepared from 2-chloroaniline and 3,3-dimethylacryloyl chloride using the procedure described for Example 5A. 1H NMR (400 MHz, CHLOROFORM-D) δ ppm 1.92 (s, 3H) 2.22 (s, 3H) 5.76 (s, 1H) 6.99 (t, J=7.82 Hz, 1H) 7.25 (t, J=7.82 Hz, 1H) 7.34 (d, J=8.06 Hz, 1H) 7.53 (s, 1H) 8.43 (d, J=7.82 Hz, 1H).

B. 8-Chloro-4,4-dimethyl-3,4-dihydro-1H-quinolin-2-one

Prepared from the title compound of Example 27A using the procedure for preparation of Example 5B. 1H NMR (400 MHz, CHLOROFORM-D) δ ppm 1.32 (s, 6H) 2.49 (s, 2H) 6.98 (t, J=7.93 Hz, 1H) 7.20 (d, J=7.81 Hz, 1H) 7.24 (dd, J=9.40, 1.34 Hz, 1H) 7.88 (s, 1H).

C. 8-Chloro-6-(2-chloro-acetyl)-4,4-dimethyl-3,4-dihydro-1H-quinolin-2-one

Prepared from the title compound of Example 27B using the procedure for preparation of Example 5C. 1H NMR (400 MHz, CHLOROFORM-D) δ ppm 1.37 (s, 6H) 2.54 (s, 2H) 4.60 (s, 2H) 7.84 (s, 1H) 7.86 (s, 1H) 8.01 (s, 1H).

D. 8-Chloro-6-(2-chloro-ethyl)-4,4-dimethyl-3,4-dihydro-1H-quinolin-2-one

Prepared from the title compound of Example 27C using the methodology described for the preparation of Example 1B. Isolated in 100% purity @ 254 nm; LCMS (APCI): 273 [M+H]⁺.

E. 6-[2-(4-Benzo[d]isothiazol-3-yl-piperazin-1-yl)-ethyl]-8-chloro-4,4-dimethyl-3,4-dihydro-1H-quinolin-2-one Hydrochloride

Prepared from the title compound of Example 27D and 3-piperazin-1-yl-benzo[d]isothiazole hydrochloride according to the procedure described for the preparation of Example 25A. Isolated in 100% purity @ 254 nm; LCMS (APCI): 455 [M+H]⁺. 1H NMR (400 MHz, DMSO-d6) δ ppm 1.22 (s, 6H) 2.37 (s, 2H) 2.99-3.07 (m, 2H) 3.33-3.50 (m, 5H) 3.57-3.67 (m, 2H) 4.02-4.12 (m, 2H) 7.20 (d, J=1.71 Hz, 1H) 7.26 (d, J=1.71 Hz, 1H) 7.42-7.47 (m, 1H) 7.54-7.59 (m, 1H) 8.09 (d, J=8.30 Hz, 1H) 8.11 (d, J=8.30 Hz, 1H) 9.55 (s, 1H) 11.01 (s, 1H). CHN: Calculated for C₂₄H₂₇N₄O₁S₁.1.20 HCl, C: 57.79%, H: 5.70%, N: 11.23%; found, C: 58.10%, H: 5.78%, N: 10.84%.

### Example 28

### 6-[2-(4-BENZO[D]ISOXAZOL-3-YL-PIPERAZIN-1-YL)-ETHYL]-8-ETHYL-4,4-DIMETHYL-3,4-DIHYDRO-1H-QUINOLIN-2-ONE

Prepared from the title compound of Example 26D and 3-piperazin-1-yl-benzo[d]isoxazole hydrochloride according to the procedure described in the preparation of Example 25A. Isolated in 100% purity @ 254 nm; LCMS (APCI): 433 [M+H]⁺. 1H NMR (400 MHz, CHLOROFORM-D) δ ppm 1.23 (t, J=7.62 Hz, 3H) 1.31 (s, 6H) 2.45 (s, 2H) 2.53 (q, J=7.55 Hz, 2H) 2.61-2.70 (m, 2H) 2.70-2.83 (m, 6H) 3.56-3.67 (m, 4H) 6.92 (d, J=1.76 Hz, 1H) 7.02 (d, J=1.76 Hz, 1H) 7.21 (ddd, J=8.06, 6.40, 1.56 Hz, 1H) 7.39 (s, 1H) 7.43-7.50 (m, 2H) 7.68 (d, J=8.01 Hz, 1H). CHN: Calculated for C₂₆H₃₂N₄O₁S₁.0.51CH2Cl2, C: 66.91%, H: 6.99%, N: 11.77%; found, C: 66.57%, H: 7.20%, N: 11.88%.

The procedure of Example 25A was employed with the title compound of Example 21D and the appropriate aryl piperazine analog to give Examples 29 through 38.

### Example 29

### 6-[2-(4-BENZO[D]ISOXAZOL-3-YL-PIPERAZIN-1-YL)-ETHYL]-4,4,8-TRIMETHYL-3,4-DIHYDRO-1H-QUINOLIN-2-ONE

With 3-piperazin-1-yl-benzo[d]isoxazole hydrochloride. Isolated in 100% purity @ 254 nm; LCMS (APCI): 419 [M+H]⁺. 1H NMR (400 MHz, CHLOROFORM-D) δ ppm 1.30 (s, 6H) 2.20 (s, 3H) 2.45 (s, 2H) 2.60-2.68 (m, 2H) 2.69-2.81 (m, 6H) 3.57-3.65 (m, 4H) 6.90 (d, J=1.22 Hz, 1H) 7.01 (d, J=1.47 Hz, 1H) 7.21 (ddd, J=8.06, 6.35,1.71 Hz, 1H) 7.33 (s, 1H) 7.43-7.50 (m, 2H) 7.68 (d, J=8.06 Hz, 1H). CHN: Calculated for C₂₅H₃₀N₄O₂, C: 71.74%, H: 7.32%, N: 13.39%; found, C: 71.30%, H: 7.14%, N: 13.11%.

### Example 30

### 6-{2-[4-(5-METHOXY-BENZO[D]ISOTHIAZOL-3-YL)-PIPERAZIN-1-YL]-ETHYL}-4,4,8-TRIMETHYL-3,4-DIHYDRO-1H-QUINOLIN-2-ONE

With 4-(5-methoxy-benzo[a]isothiazol-3-yl)-piperazine (J. Med. Chem., 1991, 34, 3316). Isolated in 100% purity @ 254 nm; LCMS (APCI): 465 [M+H]⁺. 1H NMR (400 MHz, CHLOROFORM-D) δ ppm 1.31 (s, 6H) 2.22 (s, 3H) 2.46 (s, 2H) 2.63-2.73 (m, 2H) 2.74-2.85 (m, 6H) 3.51-3.65 (m, 4H) 3.89 (s, 3H) 6.92 (s, 1H) 7.03 (d, J=1.37 Hz, 1H) 7.14 (dd, J=8.79, 2.34 Hz, 1H) 7.25 (d, J=2.54 Hz, 2H) 7.43 (s, 1H) 7.68 (d, J=8.79 Hz,1H).

### Example 31

### 6-{2-[4-(7-METHOXY-BENZO[D]ISOTHIAZOL-3-YL)-PIPERAZIN-1-YL]-ETHYL}-4,4,8-TRIMETHYL-3,4-DIHYDRO-1H-QUINOLIN-2-ONE

With 4-(7-methoxy-benzo[d]isothiazol-3-yl)-piperazine (J. Med. Chem., 1991, 34, 3316). Isolated in 100% purity @ 254 nm; LCMS (APCI): 465 [M+H]⁺. 1H NMR (400 MHz, CHLOROFORM-D) δ ppm 1.31 (s, 6H) 2.23 (s, 3H) 2.45 (s, 2H) 2.61-2.70 (m, 2H) 2.71-2.81 (m, 6H) 3.54-3.64 (m, 4H) 3.96 (s, 3H) 6.82 (d, J=7.61 Hz, 1H) 6.91 (s, 1H) 7.02 (s, 1H) 7.29 (t, J=7.91 Hz, 1H) 7.47 (d, J=7.81 Hz, 1H) 7.81 (s, 1H).

### Example 32

### 6-{2-[4-(5-FLUORO-BENZO[D]ISOTHIAZOL-3-YL)-PIPERAZIN-1-YL]-ETHYL}-4,4,8-TRIMETHYL-3,4-DIHYDRO-1H-QUINOLIN-2-ONE

With 4-(5-fluoro-benzo[d]isothiazol-3-yl)-piperazine. Isolated in 100% purity @ 254 nm; LCMS (APCI): 453 [M+H]⁺. 1H NMR (400 MHz, CHLOROFORM-D) δ ppm 1.32 (s, 6H) 2.22 (s, 3H) 2.47 (s, 2H) 2.61-2.96 (m, 8H) 3.50-3.80 (m, 4H) 6.92 (s, 1H) 7.03 (s, 1H) 7.33 (s, 1H) 7.48-7.57 (m, 1H) 7.75 (dd, J=8.91, 4.76 Hz, 1H).

### Example 33

### 6-{2-[4-(5-FLUORO-BENZO[D]ISOXAZOL-3-YL)-PIPERAZIN-1-YL]-ETHYL}-4,4,8-TRIMETHYL-3,4-DIHYDRO-1H-QUINOLIN-2-ONE

With 4-(5-fluoro-benzo[d]isoxazol-3-yl)-piperazine. Isolated in 100% purity @ 254 nm; LCMS (APCI): 437 [M+H]⁺. 1H NMR (400 MHz, CHLOROFORM-D) δ ppm 1.31 (s, 6H) 2.22 (s, 3H) 2.46 (s, 2H) 2.65 (m, 2H) 2.73 (s, 3H) 2.78 (m, 3H) 3.57 (s, 4H) 6.90 (d, J=1.22 Hz, 1H) 7.01 (d, J=1.46 Hz, 1H) 7.22 (dd, J=9.03, 2.68 Hz, 1H) 7.33 (dd, J=8.30, 2.20 Hz, 1H) 7.40 (dd, J=9.03, 3.66 Hz, 1H) 7.48 (s, 1H).

### Example 34

### 6-{2-[4-(6-FLUORO-BENZO[D]ISOXAZOL-3-YL)-PIPERAZIN-1-YL]-ETHYL}-4,4,8-TRIMETHYL-3,4-DIHYDRO-1H-QUINOLIN-2-ONE

With 4-(6-fluoro-benzo[d]isoxazol-3-yl)-piperazine (EP-494817 A1). Isolated in 100% purity @ 254 nm; LCMS (APCI): 437 [M+H]⁺. 1H NMR (400 MHz, CHLOROFORM-D) δ ppm 1.31 (s, 6H) 2.22 (s, 3H) 2.46 (s, 2H) 2.62-2.68 (m, 2H) 2.70-2.81 (m, 6H) 3.54-3.64 (m, 4H) 6.90 (d, J=1.22 Hz, 1H) 6.97 (td, J=8.78, 2.20 Hz, 1H) 7.01 (d, J=1.46 Hz, 1H) 7.13 (dd, J=8.54, 1.95 Hz, 1H) 7.52 (s, 1H) 7.63 (dd, J=8.79, 5.12 Hz, 1H).

### Example 35

### 6-{2-[4-(5-CHLORO-BENZO[D]ISOXAZOL-3-YL)-PIPERAZIN-1-YL]-ETHYL}-4,4,8-TRIMETHYL-3,4-DIHYDRO-1H-QUINOLIN-2-ONE

With 4-(5-chloro-benzo[d]isoxazol-3-yl)-piperazine (J. Med. Chem., 1986, 29, 359). Isolated in 100% purity @ 254 nm; LCMS (APCI): 453 [M+H]⁺. 1H NMR (400 MHz, CHLOROFORM-D) δ ppm 1.30 (s, 6H) 2.21 (s, 3H) 2.45 (s, 2H) 2.60-2.68 (m, 2H) 2.68-2.81 (m, 6H) 3.52-3.62 (m, 4H) 6.89 (s, 1H) 7.00 (s, 1H) 7.35-7.48 (m, 3H) 7.65 (d, J=1.71 Hz, 1H).

### Example 36

### 6-{2-[4-(7-FLUORO-BENZO[D]ISOTHIAZOL-3-YL)-PIPERAZIN-1-YL]-ETHYL}-4,4,8-TRIMETHYL-3,4-DIHYDRO-1H-QUINOLIN-2-ONE

With 4-(7-fluoro-benzo[d]isothiazol-3-yl)-piperazine. Isolated in 100% purity @ 254 nm; LCMS (APCI): 453 [M+H]⁺. 1H NMR (400 MHz, CHLOROFORM-D) δ ppm 1.31 (s, 6H) 2.21 (s, 3H) 2.46 (s, 2H) 2.61-2.69 (m, 2H) 2.71-2.81 (m, 6H) 3.54-3.64 (m, 4H) 6.91 (s, 1H) 7.02 (s, 1H) 7.10-7.18 (m, 1H) 7.29-7.40 (m, 2H) 7.68 (d, J=8.06 Hz, 1H).

### Example 37

### 6-{2-[4-(6-METHYL-BENZO[D]ISOXAZOL-3-YL)-PIPERAZIN-1-YL]-ETHYL}-4,4,8-TRIMETHYL-3,4-DIHYDRO-1H-QUINOLIN-2-ONE

With 4-(6-methyl-benzo[d]isoxazol-3-yl)-piperazine. Isolated in 100% purity @ 254 nm; LCMS (APCI): 433 [M+H]⁺. 1H NMR (400 MHz, CHLOROFORM-D) δ ppm 1.30 (s, 6H) 2.20 (s, 3H) 2.45 (s, 2H) 2.46 (s, 3H) 2.60-2.66 (m, 2H) 2.69-2.73 (m, 4H) 2.73-2.79 (m, 2H) 3.56-3.62 (m, 4H) 6.89 (d, J=1.22 Hz, 1H) 6.99-7.03 (m, 2H) 7.23 (s, 1H) 7.34 (s, 1H) 7.53 (d, J=8.30 Hz, 1H).

### Example 38

### 6-{2-[4-(6-FLUORO-BENZO[D]ISOTHIAZOL-3-YL)-PIPERAZIN-1-YL]-ETHYL}-4,4,8-TRIMETHYL-3,4-DIHYDRO-1H-QUINOLIN-2-ONE

With 4-(6-fluoro-benzo[d]isothiazol-3-yl)-piperazine (FR-2761067 A1). Isolated in 100% purity @ 254 nm; LCMS (APCI): 452 [M+H]⁺. 1H NMR (400 MHz, CHLOROFORM-D) δ ppm 1.30 (s, 6H) 1.99-2.26 (m, 10H) 2.45 (s, 2H) 2.57-2.65 (m, 4H) 2.74-2.80 (m, 2H) 3.12-3.24(m, 3H 6.90 (s, 1H) 7.01 (s, 1H) 7.15 (td, J=8.67, 2.20 Hz, 1H) 7.37 (s, 1H) 7.56 (dd, J=8.18, 2.08 Hz, 1H) 7.94 (dd, J=8.91, 4.76 Hz, 1H).

The title compound of Example 32 underwent N-alkylation as described in the preparation of Examples 39 through 44:

### Example 39

### 6-{2-[4-(5-FLUORO-BENZO[D]ISOTHIAZOL-3-YL)-PIPERAZIN-1-YL]-ETHYL}-1,4,4,8-TETRAMETHYL-3,4-DIHYDRO-1H-QUINOLIN-2-ONE HYDROCHLORIDE

To a solution of 6-{2-[4-(5-Fluoro-benzo[d]isothiazol-3-yl)-piperazin-1-yl]-ethyl}-4,4,8-trimethyl-3,4-dihydro-1H-quinolin-2-one (1 eq) in dry THF was added potassium tert-butoxide (1.5 eq) and the whole heated to 40° C. for 10 min. To the stirring solution was added iodomethane (1.5 eq) and the reaction heated to 60 in a sealed vial for 16 h. Upon cooling, the reaction was diluted with water and EtOAc and the layers separated. The aqueous was washed with EtOAc and the organics were dried (MgSO4), concentrated and the residue purified by chromatography (4% MeOH/DCM). Title product was isolated 1,4-dioxane solution upon treatment with 1N HCl ethyl ether (Et₂O) solution. Isolated in 100% purity @ 254 nm; LCMS (APCI): 467[M+H]⁺. 1H NMR (400 MHz, CHLOROFORM-D) δ ppm 1.23 (s, 6H) 2.31 (s, 3H) 2.38 (s, 2H) 3.11-3.32 (m, 8H) 3.52-3.64 (m, 2H) 4.01-4.10 (m, 2H) 4.10-4.24 (m, 2H) 6.96 (d, J=4.15 Hz, 2H) 7.30 (td, J=8.55, 2.20 Hz, 1H) 7.45 (dd, J=8.79, 2.20 Hz, 1H) 7.79 (dd, J=9.04, 4.64 Hz, 1H) 13.45 (s, 1H).

Starting with 6-{2-[4-(5-Fluoro-benzo[d]isothiazol-3-yl)-piperazin-1-yl]-ethyl}-4,4,8-trimethyl-3,4-dihydro-1H-quinolin-2-one and the appropriate alkyl halide, the title compounds of Examples 40-44 were prepared according to the procedure outlined in Example 39.

### Example 40

### 1-ETHYL-6-{2-[4-(5-FLUORO-BENZO[D]ISOTHIAZOL-3-YL)-PIPERAZIN-1-YL]-ETHYL}-4,4,8-TRIMETHYL-3,4-DIHYDRO-1H-QUINOLIN-2-ONE HYDROCHLORIDE

Isolated in 100% purity @ 254 nm; LCMS (APCI): 481[M+H]⁺. 1H NMR (400 MHz, CHLOROFORM-D) δ ppm 1.09 (t, J=7.20 Hz, 3H) 1.24 (s, 6H) 2.29 (s, 3H) 2.35 (s, 2H) 3.13-3.28 (m, 6H) 3.54-3.63 (m, 2H) 3.95 (q, J=7.08 Hz, 2H) 4.01-4.08 (m, 2H) 4.12-4.21 (m, 2H) 6.94 (d, J=1.47 Hz, 1H) 6.98 (d, J=1.95 Hz, 1H) 7.30 (td, J=8.61, 2.32 Hz, 1H) 7.44 (dd, J=8.79, 1.95 Hz, 1H) 7.79 (dd, J=8.79, 4.64 Hz, 1H) 13.40 (s, 1H).

### Example 41

### 6-{2-[4-(5-FLUORO-BENZO[D]ISOTHIAZOL-3-YL)-PIPERAZIN-1-YL]-ETHYL}-4,4,8-TRIMETHYL-1-PROPYL-3,4-DIHYDRO-1H-QUINOLIN-2-ONE HYDROCHLORIDE

Isolated in 100% purity @ 254 nm; LCMS (APCI): 495[M+H]⁺. 1H NMR (400 MHz, CHLOROFORM-D) δ ppm 0.84 (t, J=7.45 Hz, 3H) 1.24 (s, 6H) 1.48 (hextet, J=7.42 Hz, 2H) 2.29 (s, 3H) 2.35 (s, 2H) 3.13-3.28 (m, 6H) 3.59 (d, J=11.23 Hz, 2H) 3.77-3.86 (m, 2H) 4.04 (d, J=14.41 Hz, 2H) 4.11-4.12 (m, 2H) 6.93 (d, J=1.71 Hz, 1H) 6.97 (d, J=1.71 Hz, 1H) 7.30 (td, J=8.61, 2.32 Hz, 1H) 7.44 (dd, J=8.91, 2.32 Hz, 1H) 7.79 (dd, J=8.91, 4.52 Hz, 1H) 13.39 (s, 1H).

### Example 42

### 6-{2-[4-(5-FLUORO-BENZO[D]ISOTHIAZOL-3-YL)-PIPERAZIN-1-YL]-ETHYL}-1-ISOPROPYL-4,4,8-TRIMETHYL-3,4-DIHYDRO-1H-QUINOLIN-2-ONE HYDROCHLORIDE

Isolated in 100% purity @ 254 nm; LCMS (APCI): 495[M+H]⁺.

### Example 43

### 6-{2-[4-(5-FLUORO-BENZO[D]ISOTHIAZOL-3-YL)-PIPERAZIN-1-YL]-ETHYL}-1-METHOXYMETHYL-4,4,8-TRIMETHYL-3,4-DIHYDRO-1H-QUINOLIN-2-ONE HYDROCHLORIDE

Isolated in 100% purity @ 254 nm; LCMS (APCI): 497[M+H]⁺. 1H NMR (400 MHz, CHLOROFORM-D) δ ppm 1.27 (s, 6H) 2.28 (s, 3H) 2.37 (s, 2H) 3.13-3.28 (m, 8H) 3.50 (t, J=5.74 Hz, 2H) 3.59 (d, J=11.23 Hz, 2H) 4.01-4.20 (m, 6H) 6.93 (s, 1H) 6.98 (d, J=1.22 Hz, 1H) 7.30 (td, J=8.61, 2.32 Hz, 1H) 7.44 (dd, J=8.79, 2.20 Hz, 1H) 7.79 (dd, J=8.79, 4.64 Hz, 1H) 13.39 (s, 1H).

### Example 44

### 1-(2-ETHOXY-ETHYL)-6-{2-[4-(5-FLUORO-BENZO[D]ISOTHIAZOL-3-YL)-PIPERAZIN-1-YL]-ETHYL}-4,4,8-TRIMETHYL-3,4-DIHYDRO-1H-QUINOLIN-2-ONE HYDROCHLORIDE

Isolated in 100% purity @ 254 nm; LCMS (APCI): 525[M+H]⁺. 1H NMR (400 MHz, CHLOROFORM-D) δ ppm 0.94 (t, J=6.96 Hz, 3H) 1.22 (s, 6H) 2.23 (s, 3H) 2.32 (s, 2H) 3.07-3.22 (m, 6H) 3.26 (q, J=6.92 Hz, 2H) 3.47 (t, J=5.86 Hz, 2H) 3.54 (d, J=11.23 Hz, 2H) 3.96-4.16 (m, 6H) 6.87 (s, 1H) 6.93 (s, 1H) 7.25 (td, J=8.55, 2.20 Hz, 1H) 7.39 (dd, J=8.91, 2.32 Hz, 1H) 7.74 (dd, J=8.79, 4.40 Hz, 1H) 13.34 (s, 1H).

### Example 45

### 6-[2-(4-BENZO[B]THIOPHEN-3-YL-PIPERAZIN-1-YL)-ETHYL]-4S-METHYL-3,4-DIHYDRO-1H-QUINOLIN-2-ONE

Prepared from 1-benzo[b]thiophen-3-yl piperazine hydrochloride (500 mg, 1.96 mmol; J. Med. Chem., 1992, 35, 2712) and 6-(2-chloroethyl)-4S-methyl-3,4-dihydro-1H-quinolin-2-one (658 mg, 2.94 mmol) in accordance with the procedure described in Example 2. The crude product was eluted through a flash column (silica gel 60, 230-400 mesh, EtOAc) to give an orange oil which was dissolved in EtOAc and the solution treated with 4.0 N HCl in dioxane to precipitate the hydrochloride salt as an off-white, amorphous solid, yield=262 mg (30%). MS (APCI): (M+1)⁺=406; (M−1)⁺=404. ¹H-NMR (DMSO-d₆, δ): 10.51 (br s, 1H), 10.09 (s, 1H), 7.92 (d, 1H, J=6.6 Hz), 7.78 (d, 1H, J=7.8 Hz), 7.37 (m, 2H), 7.10 (d, 2H, J=8.1 Hz), 7.05 (d, 1H, J=8.3 Hz), 6.81 (d, 1H, J=8.1 Hz), 3.64 (m, 4H), 3.35 (m, 4H), 3.06 (m, 5H), 2.55 (dd, 1H, J=5.9, 6.1 Hz), 2.20 (dd, 1H, J=7.1, 7.1 Hz), 1.17 (d, 3H, J=7.1 Hz). CHN: calculated for C₂₄H₂₇N₃OS 1HCl, C: 65.22%, H: 6.38%, N: 9.51%; found, C: 64.76%, H: 6.50%, N: 9.07%. Optical rotation: [Ε]₂₅^{D}=−4.16° (DMSO, c=4.81 mg/ml).

### Example 46

### 6-[2-(4-BENZO[B]THIOPHEN-3-YL-PIPERAZIN-1-YL)-ETHYL]-4R-METHYL-3,4-DIHYDRO-1H-QUINOLIN-2-ONE

Prepared from 1-benzo[b]thiophen-3-yl piperazine hydrochloride (600 mg, 2.06 mmol; J. Med. Chem., 1992, 35, 2712) and 6-(2-chloroethyl)-4R-methyl-3,4-dihydro-1H-quinolin-2-one (692 mg, 3.09 mmol) in accordance with the procedure described in Example 3. The crude product was eluted through a flash column (silica gel 60, 230-400 mesh, EtOAc) to give a yellow, crystalline solid, yield=319 mg (38%). MS (APCI): (M+1)⁺=406; (M−1)⁺=404. ¹H-NMR (DMSO-d₆, δ): 9.98 (s, 1H), 7.87 (d, 1H, J=6.6 Hz), 7.69 (d, 1H, J=6.6 Hz), 7.33 (m, 2H), 7.04 (s, 1H), 6.98 (d, 1H, J=7.6 Hz), 6.87 (s, 1H), 6.73 (d, 1H, J=7.8 Hz), 3.03 (m, 4H), 2.65 (m, 10H), 2.17 (dd, 1H, J=7.1, 6.8 Hz), 1.14 (d, 3H, J=6.8 Hz). CHN: calculated for C₂₄H₂₇N₃OS, C: 71.08%, H: 6.71%, N: 10.36%; found, C: 70.82%, H: 6.92%, N: 10.13%. Optical rotation: [α]₂₅^{D}=+4.40° (DMSO, c=10 mg/ml). Chiral HPLC: ChiralCel OD-H, 5 μm, 250×4.6 mm; mobile phase, IPA in hexane; flow rate, 0.30 ml/min; peak RT=47.61 min (99.96%).

### Example 47

### 6-{2-[4-(6-FLUOROBENZO[B]THIOPHEN-3-YL)-PIPERAZIN-1-YL]-ETHYL}-4S-METHYL-3,4-DIHYDRO-1H-QUINOLIN-2-ONE

Prepared from 1-(6-fluorobenzo[b]thiophen-3-yl)-piperazine hydrochloride (562 mg, 2.06 mmol; J. Med. Chem., 1992, 35, 2712) and 6-(2-chloroethyl)-4S-methyl-3,4-dihydro-1H-quinolin-2-one (692 mg, 3.09 mmol) in accordance with the procedure described in Example 2. The crude product was eluted through a flash column (silica gel 60, 230-400 mesh, 2% MeOH in EtOAc) to give an oil which crystallized on standing, yield=258 mg (30%). MS (APCI): (M+1)⁺=424; (M−1)⁺=422. ¹H-NMR (CDCl₃, δ): 7.64 (m, 2H), 7.44 (d, 1H, J=8.8 Hz), 7.04 (m, 3H), 6.65 (d, 1H, J=7.8 Hz), 6.55 (s, 1H), 3.15 (m, 5H), 2.77 (m, 9H), 2.39 (dd, 1H, J=7.3, 7.3 Hz), 1.28 (d, 3H, J=7.1 Hz). CHN: calculated for C₂₄H₂₆FN₃OS, C: 68.06%, H: 6.19%, N: 9.92%; found, C: 67.80%, H: 6.12%, N: 9.57%. Optical rotation: [α]₂₅^{D}=−0.8° (CH₂Cl₂, c=5 mg/ml). Chiral HPLC: ChiralCel OD-H, 5 μm, 250×4.6 mm; mobile phase, IPA in hexane; flow rate, 0.30 ml/min; peak RT=32.41 min (99.97%).

### Example 48

### 6-{2-[4-(6-FLUOROBENZO[B]THIOPHEN-3-YL)-PIPERAZIN-1-YL]-ETHYL}-4R-METHYL-3,4-DIHYDRO-1H-QUINOLIN-2-ONE

Prepared from 1-(6-fluorobenzo[b]thiophen-3-yl)-piperazine hydrochloride (562 mg, 2.06 mmol; J. Med. Chem., 1992, 35, 2712) and 6-(2-chloroethyl)-4R-methyl-3,4-dihydro-1H-quinolin-2-one (692 mg, 3.09 mmol) in accordance with the procedure described in Example 3. The crude product was eluted through a flash column (silica gel 60, 230-400 mesh, 2% MeOH in EtOAc) to give an oil which crystallized on standing and washed with cold acetone, yield=180 mg (21%). MS (APCI): (M+1)⁺=424; (M+1)⁺=422. ¹H-NMR (CDCl₃, δ): 7.64 (m, 2H), 7.44 (d, 1H, J=8.8 Hz), 7.04 (m, 3H), 6.65 (d, 1H, J=8.1 Hz), 6.56 (s, 1H), 3.15 (m, 5H), 2.78 (m, 9H), 2.39 (dd, 1H, J=7.3, 7.3 Hz), 1.29 (d, 3H, J=7.1 Hz). CHN: calculated for C₂₄H₂₆FN₃OS, C: 68.06%, H: 6.19%, N: 9.92%; found, C: 67.86%, H: 6.18%, N: 9.78%. Optical rotation: [α]₂₅^{D}=+3.2° (CH₂Cl₂, c=5 mg/ml). Chiral HPLC: ChiralCel OD-H, 5 μm, 250×4.6 mm; mobile phase, IPA in hexane; flow rate, 0.30 ml/min; peak RT=34.51 min (99.97%).

### Example 49

### 6-[3-(4-BENZO[D]ISOTHIAZOL-3-YL-PIPERAZIN-1-YL)-PROPYL]-4,4-DIMETHYL-3,4-DIHYDRO-1H-QUINOLIN-2-ONE

A. 6-(3-Chloro-propionyl)-4,4-dimethyl-3,4-dihydro-1H-quinolin-2-one

4,4-dimethyl-3,4-dihyrdo-1H-quinolin-2-one (10 g, 57.1 mmol) was dissolved in 60 ml carbon disulfide. Aluminum chloride (15.0 g, 112 mmol) and 3-chloropropionyl chloride (7.0 mL, 84.4 mmol) was added slowly. The reaction was heated to reflux and stirred for 3 hours. The carbon disulfide was decanted off and the reaction flask cooled in an ice bath. Ice and water were slowly added until all the aluminum chloride has reacted and a precipitate had formed. The reaction mixture was stirred for 1 hour. The precipitate was filtered off and washed with ample amounts of water. 6-(3-chloro-propionyl)-4,4-dimethyl-3,4-dihydro-1H-quinolin-2-one (14.34 g) was dried in vacuo. MS (APCI): 266 [M+H]⁺.

B. 6-(3-Chloro-Propyl)-4,4-dimethyl-3,4-dihydro-1H-quinolin-2-one

6-(3-chloro-propionyl)-4,4-dimethyl-3,4-dihydro-1H-quinolin-2-one (6.0 g) was dissolved in trifluoroacetic acid (13.9 mL) and cooled to 0° C. Triethysilane (10.8 mL) was added slowly and the mixture stirred at room temperature for 3 days. The mixture was poured into ice water layered with hexanes and stirred vigorously for 30 minutes. The resultant precipitate was filtered off, washed with water and dried in vacuo to afford 6-(3-chloro-propyl)-4,4-dimethyl-3,4-dihydro-1H-quinolin-2-one. Yield 100%; MS (APCI): 252 [M+H]⁺.

C. 6-[3-(4-Benzo[d]isothiazol-3-yl-piperazin-1-yl)-propyl]-4,4-dimethyl-3,4-dihydro-1H-quinolin-2-one

Anhydrous sodium carbonate (0.160 g) was diluted in 10 mL water. 6-(3-Chloro-propyl)-4,4-dimethyl-3,4-dihydro-1H-quinolin-2-one (0.300 g, 1.19 mmol), 3-piperazin-1-yl-benzisothiazole (0.390 g, 1.78 mmol), and acetonitrile (10 mL) was added. The mixture stirred at reflux for 48 hours. After cooling for 1 hour, the solution was diluted with ethyl acetate and washed with water. The organic extracts were dried over sodium sulfate (Na₂SO₄), concentrated and dried in vacuo to afford 6-[3-(4-benzo[d]isothiazol-3-yl-piperazin-1-yl)-propyl]-4,4-dimethyl-3,4-dihydro-1H-quinolin-2-one ( 0.030 g). MS (APCI): 435 [M+H]⁺. ¹H NMR (400 MHz, CDCl₃) δ 8.23 (s, 1H), 7.85 (d, J=8.1 Hz, 1H), 7.76 (d, J=8.1 Hz, 1H), 7.4 (t, J=8.1 Hz, 1H), 7.29 (t, J=8.1 Hz, 1H), 7.09 (s, 1H), 6.95 (dd, J=1.7,1.9 Hz, 1H), 6.66 (d, J=7.81 Hz, 1H), 3.55 (s, 4H), 2.66 (s, 4H), 2.58 (t, J=7.5, 7.8 Hz, 2H), 2.44 (s, 4H), 1.81 (m, 2H), 1.28 (s, 6H).

### Example 50

### 6-{3-[4-(1H-INDAZOL-3-YL)-PIPERAZIN-1-YL]-PROPYL}-4,4-DIMETHYL-3,4-DIHYDRO-1H-QUINOLIN-2-ONE

6-{3-[4-(1H-indazol-3-yl)-piperazin-1-yl]-propyl}-4,4-dimethyl-3,4-dihydro-1H-quinolin-2-one was prepared according to the general procedure outlined in Example 49C, starting with anhydrous sodium carbonate (2.50 g), 6-(3-chloro-propyl)-4,4-dimethyl-3,4-dihydro-1H-quinolin-2-one (2.0 g, 7.94 mmol) and 3-piperazin-1-yl-1H-indazole hydrochloride (2.0 g, 8.38 mmol). The solid was purified using an ISCO autocolumn eluting with 80% ethyl acetate in hexanes to afford 0.200 g of 6-{3-[4-(1H-indazol-3-yl)-piperazin-1-yl]-propyl}-4,4-dimethyl-3,4-dihydro-1H-quinolin-2-one. MS (APCI): 418 [M+H]⁺. ¹H NMR (400 MHz, CDCl₃) δ 9.13 (s, 1H), 7.66 (d, J=8.3 Hz, 1H), 7.58 (s, 1H), 7.3 (s, 1H), 7.0 (m, 3H), 6.62 (d, J=7.81 Hz, 1H), 3.4 (s, 4H), 2.6 (m, 5H), 2.44 (s, 3H), 1.84 (m, 2H), 1.54 (s, 2H), 1.28 (s, 6H).

### Example 51

### 6-[3-(4-BENZO[D]ISOTHIAZOL-3-YL-PIPERAZIN-1-YL)-PROPYL]-7-CHLORO-4,4-DIMETHYL-3,4-DIHYDRO-1H-QUINOLIN-2-ONE

A. 7-chloro-6-(3-chloro-propionyl)-4,4-dimethyl-3,4-dihydro-1H-quinolin-2-one

7-Chloro-6-(3-chloro-propionyl)-4,4-dimethyl-3,4-dihydro-1H-quinolin-2-one was prepared according to the general procedure outlined in step A of Example 49, starting with 7-chloro-4,4-dimethyl-3,4-dihyrdo-1H-quinolin-2-one (1.00 g, 4.77 mmol), aluminum chloride (2.54 g, 19.1 mmol) and 3-chloropropionyl chloride (0.47 mL, 5.66 mmol). 7-Chloro-6-(3-chloro-propionyl)-4,4-dimethyl-3,4-dihydro-1H-quinolin-2-one (1.09 g) was afforded in 76% yield. MS (APCI): 300 [M+H]⁺.

B. 7-chloro-6-(3-chloro-propyl)-4,4-dimethyl-3,4-dihydro-1H-quinolin-2-one

7-Chloro-6-(3-chloro-propyl)-4,4-dimethyl-3,4-dihydro-1H-quinolin-2-one was prepared according to the general procedure outlined in step B of Example 49, starting with 7-chloro-6-(3-chloro-propionyl)-4,4-dimethyl-3,4-dihydro-1H-quinolin-2-one (1.05 g, 3.49 mmol), trifluoroacetic acid (1.86 mL, 24.1 mmol) and triethysilane (0.939 mL, 5.88 mmol). 7-chloro-6-(3-chloro-propoyl)-4,4-dimethyl-3,4-dihydro-1H-quinolin-2-one (0.260 g) was afforded in 26% yield. MS (APCI): 286 [M+H]⁺.

C. 6-[3-(4-Benzo[d]isothiazol-3-yl-piperazin-1-yl)-propyl]-7-chloro-4,4-dimethyl-3,4-dihydro-1H-quinolin-2-one

6-[3-(4-Benzo[d]isothiazol-3-yl-piperazin-1-yl)-propyl]-7-chloro-4,4-dimethyl-3,4-dihydro-1H-quinolin-2-one was prepared according to the general procedure outlined in Example 49C, starting with anhydrous sodium carbonate (0.097 g), 7-chloro-6-(3-chloro-propyl)-4,4-dimethyl-3,4-dihydro-1H-quinolin-2-one (0.200 g, 0.698 mmol) and 3-piperazin-1-yl-benzo[d]isothiazole hydrochloride (0.229 g, 1.04 mmol). The solid was purified using an ISCO autocolumn eluting with 80% ethyl acetate in hexanes to afford 0.084 g of 7-chloro-6-[3-(4-1,2-Benzisothiazol-3-yl-piperazin-1-yl)-propyl]-4,4-dimethyl-3,4-dihydro-1H-quinolin-2-one. 100% purity @ 254 nm; LCMS (APCI) 469 [M+H]⁺. ¹H NMR (400 MHz, CDCl₃) δ 8.16 (s, 1H), 7.85 (d, J=8.1 Hz, 1H), 7.76 (d, J=8.3 Hz, 1H), 7.4 (t, J=8.1 Hz, 1H), 7.3 (t, J=8.1 Hz, 1H), 7.1 (s, 1H), 6.76 (s, 1H), 3.57 (s, 4H), 2.7 (m, 6H, 2.45 (t, J=7.1, 7.5 Hz, 3H), 2.43 (s, 1H), 1.8 (m, 2H), 1.27 (s, 6H).

### Example 52

### 6-[3-(4-BENZO[D]ISOXAZOL-3-YL-PIPERAZIN-1-YL)-PROPYL]-7-CHLORO-4,4-DIMETHYL-3,4-DIHYDRO-1H-QUINOLIN-2-ONE

6-[3-(4-Benzo[d]isoxazol-3-yl-piperazin-1-yl)-propyl]-7-chloro-4,4-dimethyl-3,4-dihydro-1H-quinolin-2-one was prepared according to the general procedure outlined in Example 49, starting with anhydrous sodium carbonate (0.022 g), 7-chloro-6-(3-chloro-propyl)-4,4-dimethyl-3,4-dihydro-1H-quinolin-2-one (0.046 g, 0.162 mmol) and 3-piperazin-1-yl-benzo[d]isoxazole (0.033 g, 0.162 mmol). The solid was purified using an ISCO autocolumn eluting with 80% ethyl acetate in hexanes to afford 0.014 g of 7-chloro-6-[3-(4-1,2-Benzisoxazol-3-yl-piperazin-1-yl)-propyl]-4,4-dimethyl-3,4-dihydro-1H-quinolin-2-one. MS (APCI): 453 [M+H]⁺. ¹H NMR (400 MHz, CDCl₃) δ 8.23 (s, 1H), 7.6 (d, J=8.1 Hz, 1H), 7.39 (t, J=7.8, 8.5 Hz, 2H), 7.13 (t, J=6.8, 7.8 Hz, 1H), 7.07 (s, 1H), 6.74 (s, 1H), 3.5 (s, 4H), 2.66 (m, 6H), 2.6 (s, 4H), 1.77 (m, 2H), 1.25 (s, 6H).

### Example 53

### 6-[3-(4-BENZO[D]ISOTHIAZOL-3-YL-PIPERAZIN-1-YL)-PROPYL]-4-METHYL-3,4-DIHYDRO-1H-QUINOLIN-2-ONE

A. 6-(3-Chloro-propionyl)-4-methyl-3,4-dihydro-1H-quinolin-2-one

6-(3-Chloro-propionyl)-4-methyl-3,4-dihydro-1H-quinolin-2-one was prepared according to the general procedure outlined in step A of Example 49, starting with 4-methyl-3,4-dihyrdo-1H-quinolin-2-one (10.0 g, 62.0 mmol), aluminum chloride (15.0 g, 112.5 mmol) and 3-chloropropionyl chloride (7.2 mL, 86.8 mmol). 6-(3-Chloro-propionyl)-4-methyl-3,4-dihydro-1H-quinolin-2-one (7.79 g) was afforded in 50% yield. MS (APCI): 251 [M+H]⁺.

B. 6-(3-Chloro-propyl)-4-methyl-3,4-dihydro-1H-quinolin-2-one

6-(3-Chloro-propyl)-4-methyl-3,4-dihydro-1H-quinolin-2-one was prepared according to the general procedure outlined in step B of Example 49, starting with 6-(3-chloro-propionyl)-4-methyl-3,4-dihydro-1H-quinolin-2-one (5.0 g, 19.8 mmol), trifluoroacetic acid (9.0 mL, 116.8 mmol) and triethysilane (9.52 mL, 59.6 mmol). 6-(3-Chloro-propyl)-4-methyl-3,4-dihydro-1H-quinolin-2-one was afforded in 100% yield. MS (APCI): 238 [M+H]⁺.

C. 6-[3-(4-Benzo[d]isothiazol-3-yl-piperazin-1-yl)-propyl]-4-methyl-3,4-dihydro-1H-quinolin-2-one

6-[3-(4-Benzo[d]isothiazol-3-yl-piperazin-1-yl)-propyl]-4-methyl-3,4-dihydro-1H-quinolin-2-one was prepared according to the general procedure outlined in Example 49C, starting with anhydrous sodium carbonate (0.50 g), 6-(3-chloro-propyl)-4-methyl-3,4-dihydro-1H-quinolin-2-one (0.300 g, 1.42 mmol) and 3-piperazin-1-yl-benzo[d]isothiazole hydrochloride (0.62 g, 2.83 mmol). The resultant precipitate was washed with ample amounts of water and acetonitrile and dried in vacuo to afford 0.315 g of 6-[3-(4-1,2-benzisothiazol-3-yl-piperazin-1-yl)-propyl]-4-methyl-3,4-dihydro-1H-quinolin-2-one. 100% purity @ 254 nm; LCMS (APCI) 421 [M+H]⁺. ¹H NMR (400 MHz, CDCl₃) δ 8.36 (s, 1H), 7.85 (d, J=8.1 Hz, 1H), 7.76 (d, J=8.1 Hz, 1H), 7.4 (t, J=7.56 Hz, 1H), 7.3 (t, J=7.3, 7.81 Hz, 1H), 7.0 (s, 1H), 6.96 (d, J=8.6 Hz, 1H), 6.67 (d, J=8.1 Hz, 1H), 3.5 (t, J=4.39, 4.88 Hz, 4H), 3.04 (m, 1H), 2.6 (m, 7H), 2.35 (m, 3H), 1.8 (m, 2H), 1.26 (d, J=7.1 Hz, 3H).

### Example 54

### 6-[3-(4-BENZO[D]ISOXAZOL-3-YL-PIPERAZIN-1-YL)-PROPYL]-4-METHYL-3,4-DIHYDRO-1H-QUINOLIN-2-ONE

6-[3-(4-Benzo[d]isoxazol-3-yl-piperazin-1-yl)-propyl]-4-methyl-3,4-dihydro-1H-quinolin-2-one was prepared according to the general procedure outlined in Example 49, starting with anhydrous sodium carbonate (0.450 g), 6-(3-chloro-propyl)-4-methyl-3,4-dihydro-1H-quinolin-2-one (0.300 g, 1.42 mmol) and 3-piperazin-1-yl-benzo[d]isoxazole (0.578 g, 2.84 mmol). The solid was purified using an ISCO autocolumn eluting with 80% ethyl acetate in hexanes to afford 0.185 g of 6-[3-(4-1,2-Benzisoxazol-3-yl-piperazin-1-yl)-propyl]-4-methyl-3,4-dihydro-1H-quinolin-2-one. 100% purity @ 254 nm; LCMS (APCI) 405 [M+H]⁺. ¹H NMR (400 MHz, CDCl₃) δ 7.95 (s, 1H), 7.6 (d, J=8.1 Hz, 2H), 7.4 (m, 2H), 7.15 (m, 1H), 6.99 (s, 1H), 6.96 (d, J=8.1 Hz, 1H), 6.64 (d, J=8.1 Hz, 1H), 3.5 (t, J=4.8, 5.1 Hz, 3H), 3.09 (m, 1H), 2.66 (m, 8H), 2.4 (m, 3H), 1.8 (m, 2H), 1.26 (d, J=6.83 Hz, 3H).

### Example 55

### 6-{3-[4-(1H-INDAZOL-3-YL)-PIPERAZIN-1-YL]-PROPYL}-4-METHYL-3,4-DIHYDRO-1H-QUINOLIN-2-ONE

6-{3-[4-(1H-Indazol-3-yl)-piperazin-1-yl]-propyl}-4-methyl-3,4-dihydro-1H-quinolin-2-one was prepared according to the general procedure outlined in Example 49C, starting with anhydrous sodium carbonate (5.79 g), 6-(3-chloro-propyl)-4-methyl-3,4-dihydro-1H-quinolin-2-one (3.73 g, 15.7 mmol) and 3-piperazin-1-yl-1H-indazole hydrochloride (2.5 g, 10.5 mmol). The solid was purified using an ISCO autocolumn eluting with 80% ethyl acetate in hexanes to afford 0.547 g of 6-{3-[4-(1H-indazol-3-yl)-piperazin-1-yl]-propyl}-4-methyl-3,4-dihydro-1H-quinolin-2-one. 100% purity @ 254 nm; LCMS (APCI) 404 [M+H]⁺. ¹H NMR (400 MHz, CDCl₃) δ 9.31 (s, 1H), 7.91 (s, 1H), 7.67 (d, J=8.3 Hz, 1H), 7.3 (m, 2H), 7.0 (m, 3H), 6.6 (d, J=7.81 Hz, 1H), 3.4 (m, 5H), 3.04 (m, 2H), 2.6 (t, J=4.4, 5.6 Hz, 3H), 2.57 (t, J=7.56, 7.81 Hz, 2H), 2.39 (m, 3H), 1.8 (m, 2H), 1.26 (d, J=7.1 Hz, 3H).

### Example 56

### 6-[3-(4-BENZO[D]ISOTHIAZOL-3-YL-PIPERAZIN-1-YL)-PROPYL]-3,3-DIMETHYL-3,4-DIHYDRO-1H-QUINOLIN-2-ONE

A. 6-(3-Chloro-propionyl)-3,3-dimethyl-3,4-dihydro-1H-quinolin-2-one

6-(3-Chloro-propionyl)-3,3-dimethyl-3,4-dihyd ro-1H-quinolin-2-one was prepared according to the general procedure outlined in step A of Example 49, starting with 3,3-dimethyl-3,4-dihyrdo-1H-quinolin-2-one (4.0 g, 15.89 mmol), aluminum chloride (6.4 g, 48 mmol) and 3-chloropropionyl chloride (1.85 mL, 22.3 mmol). 6-(3-chloro-propionyl)-3,3-dimethyl-3,4-dihydro-1H-quinolin-2-one was afforded in 100% yield. MS (APCI): 266 [M+H]⁺.

B. 6-(3-Chloro-propyl)-3,3-dimethyl-3,4-dihydro-1H-quinolin-2-one

6-(3-Chloro-propyl)-3,3-dimethyl-3,4-dihydro-1H-quinolin-2-one was prepared according to the general procedure outlined in step B of Example 49, starting with 6-(3-chloro-propionyl)-3,3-dimethyl-3,4-dihydro-1H-quinolin-2-one (5.0 g, 18.8 mmol), trifluoroacetic acid (10.1 mL, 131 mmol) and triethysilane (9.0 mL, 56.3 mmol). 6-(3-chloro-propyl)-3,3-dimethyl-3,4-dihydro-1H-quinolin-2-one (4.99 g) was afforded in 100% yield. MS (APCI): 252 [M+H]⁺.

C. 6-[3-(4-Benzo[d]isothiazol-3-yl-piperazin-1-yl)-propyl]-3,3-dimethyl-3,4-dihydro-1H-quinolin-2-one

6-[3-(4-Benzo[d]isothiazol-3-yl-piperazin-1-yl)-propyl]-3,3-dimethyl-3,4-dihydro-1H-quinolin-2-one was prepared according to the general procedure outlined in Example 49C, starting with anhydrous sodium carbonate (0.357 g), 6-(3-chloro-propyl)-3,3-dimethyl-3,4-dihydro-1H-quinolin-2-one (0.500 g, 1.99 mmol) and 3-piperazin-1-yl-benzo[d]isothiazole hydrochloride (0.566 g, 2.58 mmol). The resultant precipitate was washed with ample amounts of water and acetonitrile and dried in vacuo to afford 0.0991 g of 6-[3-(4-1,2-Benzisothiazol-3-yl-piperazin-1-yl)-propyl]-3,3-dimethyl-3,4-dihydro-1H-quinolin-2-one. 100% purity @ 254 nm; LCMS (APCI) 435 [M+H]⁺. ¹H NMR (400 MHz, CDCl₃) δ 7.85 (d, J=8.1 Hz, 1H), 7.76 (d, J=8.1 Hz, 1H), 7.61 (s, 1H), 7.4 (t, J=7.1, 7.3 Hz, 1H), 7.3 (t, J=7, 8.1 Hz, 1H), 6.97 (s, 1H), 6.95 (s, 1H), 6.59 (d, J=7.81 Hz, 1H), 3.5 (t, J=4.6, 4.8 Hz, 4H), 2.73 (s, 2H), 2.6 (t, J=4.6, 4.88 Hz, 3H), 2.5 (t, J=7.5, 7.8 Hz, 3H), 2.4 (t, J=7.3, 7.56 Hz, 2H), 1.8 (m, 2H), 1.16 (s, 6H).

### Example 57

### 6-[3-(4-BENZO[D]ISOXAZOL-3-YL-PIPERAZIN-1-YL)-PROPYL]-3,3-DIMETHYL-3,4-DIHYDRO-1H-QUINOLIN-2-ONE

6-[3-(4-Benzo[d]isoxazol-3-yl-piperazin-1-yl)-propyl]-3,3-dimethyl-3,4-dihydro-1H-quinolin-2-one was prepared according to the general procedure outlined in Example 49C, starting with anhydrous sodium carbonate (0.358 g), 6-(3-chloro-propyl)-3,3-dimethyl-3,4-dihydro-1H-quinolin-2-one (0.500 g, 1.99 mmol) and 3-piperazin-1-yl-benzo[d]isoxazole (0.525 g, 2.58 mmol). The solid was purified using an ISCO autocolumn eluting with 80% ethyl acetate in hexanes to afford 0.144 g of 6-[3-(4-1,2-Benzisoxazol-3-yl-piperazin-1-yl)-propyl]-3,3-dimethyl-3,4-dihydro-1H-quinolin-2-one. 100% purity @ 254 nm; LCMS (APCI) 419 [M+H]⁺. ¹H NMR (400 MHz, CDCl₃) δ 7.6 (m, 2H), 7.4 (m, 2H), 7.1 (m, 1H), 6.96 (s, 1H), 6.94 (s, 1H), 6.6 (d, J=7.81 Hz, 1H), 3.5 (t, J=4.88 Hz, 4H), 2.73 (s, 2H), 2.5-2.6 (m, 6H), 2.4 (t, J=7.3, 7.5 Hz, 2H), 1.8 (m, 2H), 1.16 (s, 6H).

### Example 58

### 6-{3-[4-(1H-INDAZOL-3-YL)-PIPERAZIN-1-YL]-PROPYL}-3,3-DIMETHYL-3,4-DIHYDRO-1H-QUINOLIN-2-ONE

6-{3-[4-(1H-indazol-3-yl)-piperazin-1-yl]-propyl}-3,3-dimethyl-3,4-dihydro-1H-quinolin-2-one was prepared according to the general procedure outlined in Example 23, starting with anhydrous sodium carbonate (6.0 g), 6-(3-chloro-propyl)-3,3-dimethyl-3,4-dihydro-1H-quinolin-2-one (1.69 g, 6.71 mmol) and 3-piperazin-1-yl-1H-indazole hydrochloride (2.0 g, 8.38 mmol). The solid was purified using an ISCO autocolumn eluting with 80% ethyl acetate in hexanes to afford 0.308 g of 6-{3-[4-(1H-indazol-3-yl)-piperazin-1-yl]-propyl}-3,3-dimethyl-3,4-dihydro-1H-quinolin-2-one. 100% purity @ 254 nm; LCMS (APCI) 418 [M+H]⁺. ¹H NMR (400 MHz, CDCl₃) δ 9.26 (s, 1H), 7.7 (d, J=8.3 Hz, 1H), 7.5 (s, 1H), 7.3 (m, 2H), 7-7.2 (m, 1H), 6.97 (s, 1H), 6.95 (s, 1H), 6.59 (d, J=7.8 Hz, 1H), 3.47 (s, 4H), 2.73 (s, 2H), 2.65 (s, 3H), 2.6 (t, J=7.8 Hz, 2H), 2.4 (s, 2H), 1.8 (s, 2H), 1.59 (s, 1H), 1.16 (s, 6H).

### Example 59

### 6-[3-(4-BENZO[D]ISOTHIAZOL-3-YL-PIPERAZIN-1-YL)-PROPYL]-3-METHYL-3,4-DIHYDRO-1H-QUINOLIN-2-ONE

A. 6-(3-Chloro-propionyl)-3-methyl-3,4-dihydro-1H-quinolin-2-one

6-(3-Chloro-propionyl)-3-methyl-3,4-dihydro-1H-quinolin-2-one was prepared according to the general procedure outlined in step A of Example 49, starting with 3-methyl-3,4-dihyrdo-1H-quinolin-2-one (10.0 g, 662 mmol), aluminum chloride (16 g,120 mmol) and 3-chloropropionyl chloride (7.20 mL, 86.7 mmol). 6-(3-Chloro-propionyl)-3-methyl-3,4-dihydro-1H-quinolin-2-one was afforded in 100% yield. MS (APCI): 252 [M+H]⁺.

B. 6-(3-Chloro-propyl)-3-methyl-3,4-dihydro-1H-quinolin-2-one

6-(3-Chloro-propyl)-3-methyl-3,4-dihydro-1H-quinolin-2-one was prepared according to the general procedure outlined in step B of Example 49, starting with 6-(3-chloro-propionyl)-3-methyl-3,4-dihydro-1H-quinolin-2-one (5.50 g, 21.8 mmol), trifluoroacetic acid (10.5 mL, 136 mmol) and triethysilane (9.0 mL, 56.0 mmol). 6-(3-chloro-propyl)-3-methyl-3,4-dihydro-1H-quinolin-2-one (4.99 g) was afforded in 100% yield. MS (APCI): 238 [M+H]⁺.

C. 6-[3-(4-Benzo[d]isothiazol-3-yl-piperazin-1-yl)-propyl]-3-methyl-3,4-dihydro-1H-quinolin-2-one

6-[3-(4-Benzo[d]isothiazol-3-yl-piperazin-1-yl)-propyl]-3-methyl-3,4-dihydro-1H-quinolin-2-one was prepared according to the general procedure outlined in Example 49C, starting with anhydrous sodium carbonate (2.33 g), 6-(3-chloro-propyl)-3-methyl-3,4-dihydro-1H-quinolin-2-one (2.00 g, 8.41 mmol) and 3-piperazin-1-yl-benzo[d]isothiazole hydrochloride (2.33 g, 16.8 mmol). The resultant precipitate was washed with ample amounts of water and acetonitrile and dried in vacuo to afford 0.452 g of 6-[3-(4-1,2-Benzisothiazol-3-yl-piperazin-1-yl)-propyl]-3-methyl-3,4-dihydro-1H-quinolin-2-one. 100% purity @ 254 nm; LCMS (APCI) 421 [M+H]⁺; m.pt. 212° C. ¹H NMR (400 MHz, CDCl₃) δ 7.91 (s, 1H), 7.86 (d, J=8.3 Hz, 1H), 7.76 (d, J=8.05 Hz, 1H), 7.40 (t, J=7.32, 7.56 Hz, 1H), 7.30 (t, J=7.32, 7.56 Hz, 1H), 6.97 (s, 2H), 6.63 (d, J=8.30 Hz, 1H), 3.54 (s, 4H), 2.9 (dd, J=5.13, 4.88 Hz, 1H), 2.56-2.71 (m, 8H), 2.4 (t, J=7.08, 8 Hz, 2H), 1.8-1.85 (m, 2H), 1.24 (d, J=6.58 Hz, 3H).

### Example 60

### 6-[3-(4-BENZO[D]ISOXAZOL-3-YL-PIPERAZIN-1-YL)-PROPYL]-3-METHYL-3,4-DIHYDRO-1H-QUINOLIN-2-ONE

6-[3-(4-Benzo[d]isoxazol-3-yl-piperazin-1-yl)-propyl]-3-methyl-3,4-dihydro-1H-quinolin-2-one was prepared according to the general procedure outlined in Example 49C, starting with anhydrous sodium carbonate (0.68 g), 6-(3-chloro-propyl)-3-methyl-3,4-dihydro-1H-quinolin-2-one (1.17 g, 4.92 mmol) and 3-piperazin-1-yl-benzo[d]isoxazole (1.30 g, 6.39 mmol). The residue was extracted with dichloromethane, dried over sodium sulfate (Na₂SO₄) and concentrated to afford 0.208 g of 6-[3-(4-1,2-Benzisoxazol-3-yl-piperazin-1-yl)-propyl]-3-methyl-3,4-dihydro-1H-quinolin-2-one. 100% purity @ 254 nm; LCMS (APCI) 405 [M+H]+; m.pt. 185-187° C. ¹H NMR (400 MHz, CDCl₃) δ 7.66 (s, 1H), 7.64 (s, 1H), 7.4 (m, 2H), 7.16 (m, 1H), 6.97 (s, 2H), 6.6 (m, 1H), 3.5 (t, J=4.39 Hz, 4H), 2.9 (dd, J=5.13, 5.37 Hz, 1H), 2.5-2.7 (m, 8H), 2.4 (t, J=7.3, 7.5 Hz, 2H), 1.7-1.8 (m, 2H), 1.24 (d, J=6.58 Hz, 3H).

### Example 61

### 6-{3-[4-(1H-INDAZOL-3-YL)-PIPERAZIN-1-YL]-PROPYL}-3-METHYL-3,4-DIHYDRO-1H-QUINOLIN-2-ONE

6-{3-[4-(1H-indazol-3-yl)-piperazin-1-yl]-propyl}-3-methyl-3,4-dihydro-1H-quinolin-2-one was prepared according to the general procedure outlined in Example 49C, starting with anhydrous sodium carbonate (7.0 g), 6-(3-chloro-propyl)-3-methyl-3,4-dihydro-1H-quinolin-2-one (3.73 g, 15.7 mmol) and 3-piperazin-1-yl-1H-indazole hydrochloride (2.5 g, 10.47 mmol). The solid was purified using an ISCO autocolumn eluting with 80% ethyl acetate in hexanes to afford 0.74 g of 6-{3-[4-(1H-indazol-3-yl)-piperazin-1-yl]-propyl}-3-methyl-3,4-dihydro-1H-quinolin-2-one. 100% purity @ 254 nm; LCMS (APCI) 404 [M+H]⁺. ¹H NMR (400 MHz, DMSO-d₆) δ 11.9 (s, 1H), 9.9 (s, 1H), 7.6 (d, J=8.05 Hz, 1H), 7.28 (d, J=8.30 Hz, 1H), 7.19 (m, 3H), 6.88 (m, 3H), 6.68 (d, J=7.81, 1H), 3.26 (s, 4H), 2.80 (dd, J=5.86, 6.0 Hz, 1H), 2.4-2.58(m, 8H), 2.27 (t, J=7.08 Hz, 2H), 1.66 (t, J=7.08, 7.32 Hz), 1.04 (d, J=6.59 Hz, 3H).

### Example 62

### 6-[3-(4-BENZO[D]ISOTHIAZOL-3-YL-PIPERAZIN-1-YL)-PROPYL]-4S-METHYL-3,4-DIHYDRO-1H-QUINOLIN-2-ONE

6-[3-(4-Benzo[d]isothiazol-3-yl-piperazin-1-yl)-propyl]-4S-methyl-3,4-dihydro-1H-quinolin-2-one was prepared according to the general procedure outlined in Example 49C, starting with anhydrous potassium carbonate (0.754 g), 6-(3-chloro-propyl)-4S-methyl-3,4-dihydro-1H-quinolin-2-one (0.4318 g, 1.82 mmol. Prepared from 4S-methyl-3,4-dihydro-1H-quinolin-2-one in U.S. Pat. No. 5,350,747) and 3-piperazin-1-yl-benzo[d]isothiazole hydrochloride (0.597 g, 2.72 mmol). The resultant precipitate was washed with ample amounts of water and acetonitrile and dried in vacuo to afford 0.600 g of 6-[3-(4-benzo[d]isothiazol-3-yl-piperazin-1-yl)-propyl]-4S-methyl-3,4-dihydro-1H-quinolin-2-one. 100% purity @ 254 nm; LCMS (APCI) 421.2 [M+H]⁺. CHN: calculated for C₂₄H₂₈N₄O₁S₁ C: 68.54%, H: 6.71, N: 13.32%; found, C: 68.07%, H: 6.78%, N: 12.86%.

### Example 63

### 6-[3-(4-BENZO[D]ISOTHIAZOL-3-YL-PIPERAZIN-1-YL)-PROPYL]-4R-METHYL-3,4-DIHYDRO-1H-QUINOLIN-2-ONE

6-[3-(4-Benzo[d]isothiazol-3-yl-piperazin-1-yl)-propyl]-4R-methyl-3,4-dihydro-1H-quinolin-2-one was prepared according to the general procedure outlined in Example 49C, starting with anhydrous potassium carbonate (0.873 g), 6-(3-chloro-propyl)-4R-methyl-3,4-dihydro-1H-quinolin-2-one (0.500 g, 2.10 mmol. Prepared from 4S-methyl-3,4-dihydro-1H-quinolin-2-one in U.S. Pat. No. 5,350,747) and 3-piperazin-1-yl-benzo[d]isothiazole hydrochloride (0.692 g, 3.16 mmol). The resultant precipitate was washed with ample amounts of water and acetonitrile and dried in vacuo to afford 0.256 g of 6-[3-(4-1,2-benzisothiazol-3-yl-piperazin-1-yl)-propyl]-4R-methyl-3,4-dihydro-1H-quinolin-2-one. 100% purity @ 254 nm; LCMS (APCI) 421.2 [M+H]⁺. CHN: calculated for C₂₄H₂₈N₄O₁S₁ C: 68.54%, H: 6.71, N: 13.32%; found, C: 68.24%, H: 6.80%, N: 13.01%.

### Example 64

### 6-[3-(4-BENZO[D]ISOXAZOL-3-YL-PIPERAZIN-1-YL)-PROPYL]-4R-METHYL-3,4-DIHYDRO-1H-QUINOLIN-2-ONE

6-[3-(4-Benzo[d]isoxazol-3-yl-piperazin-1-yl)-propyl]-4R-methyl-3,4-dihydro-1H-quinolin-2-one was prepared according to the general procedure outlined in Example 49C, starting with anhydrous potassium carbonate (0.762 g), 6-(3-chloro-propyl)-4R-methyl-3,4-dihydro-1H-quinolin-2-one (0.1308 g, 0.550 mmol) and 3-piperazin-1-yl-benzo[d]isoxazole (0.264 g, 1.10 mmol). The solid was purified using an ISCO autocolumn eluting with 80% ethyl acetate in hexanes to afford 0.027 g of 6-[3-(4-1,2-Benzisoxazol-3-yl-piperazin-1-yl)-propyl]-4R-methyl-3,4-dihydro-1H-quinolin-2-one. MS (APCI): 405.2 [M+H]⁺.

### Example 65

### 6-[3-(4-BENZO[D]ISOXAZOL-3-YL-PIPERAZIN-1-YL)-PROPYL]-4S-METHYL-3,4-DIHYDRO-1H-QUINOLIN-2-ONE

6-[3-(4-Benzo[d]isoxazol-3-yl-piperazin-1-yl)-propyl]-4S-methyl-3,4-dihydro-1H-quinolin-2-one was prepared according to the general procedure outlined in Example 49C, starting with anhydrous potassium carbonate (0.537 g), 6-(3-chloro-propyl)-4S-methyl-3,4-dihydro-1H-quinolin-2-one (0.0923 g, 0.388 mmol) and 3-piperazin-1-yl-benzo[d]isoxazole (0.186 g, 0.776 mmol). The solid was purified using an ISCO autocolumn eluting with 80% ethyl acetate in hexanes to afford 0.049 g of 6-[3-(4-1,2-Benzisoxazol-3-yl-piperazin-1-yl)-propyl]-4S-methyl-3,4-dihydro-1H-quinolin-2-one. MS (APCI): (M+H)+405.2. ¹H NMR (400 MHz, CDCl₃) δ

### Example 66

### 6-[3-(4-BENZO[D]ISOTHIAZOL-3-YL-PIPERAZIN-1-YL)-PROPYL]-7-FLUORO-4,4-DIMETHYL-3,4-DIHYDRO-1H-QUINOLIN-2-ONE

A. 6-(3-Chloro-propionyl)-7-fluoro-4,4-dimethyl-3,4-dihydro-1H-quinolin-2-one

6-(3-Chloro-propionyl)-7-fluoro-4,4-dimethyl-3,4-dihydro-1H-quinolin-2-one was prepared according to the general procedure outlined in step A of Example 49, starting with 7-fluoro-4,4-dimethyl-3,4-dihyrdo-1H-quinolin-2-one (1.00 g, 5.18 mmol), aluminum chloride (2.76 g, 20.7 mmol) and 3-chloropropionyl chloride (0.644 mL, 7.76 mmol). 6-(3-chloro-propionyl)-7-fluoro-4,4-dimethyl-3,4-dihydro-1H-quinolin-2-one (1.23 g) was afforded in 84% yield. MS (APCI): 284.1 [M+H]⁺.

B. 6-(3-Chloro-propyl)-7-fluoro-4,4-dimethyl-3,4-dihydro-1H-quinolin-2-one

6-(3-Chloro-propyl)-7-fluoro-4,4-dimethyl-3,4-dihydro-1H-quinolin-2-one was prepared according to the general procedure outlined in step B of Example 49, starting with 6-(3-chloro-propionyl)-7-fluoro-4,4-dimethyl-3,4-dihydro-1H-quinolin-2-one (1.23 g, 4.34 mmol), trifluoroacetic acid (2.09 mL, 25.9 mmol) and triethysilane (1.73 mL, 10.8 mmol). 6-(3-chloro-propyl)-7-fluoro-4,4-dimethyl-3,4-dihydro-1H-quinolin-2-one (1.15 g) was afforded in 98% yield. MS (APCI): 270.1 [M+H]⁺.

C. -[3-(4-Benzo[d]isothiazol-3-yl-piperazin-1-yl-propyl]-7-fluoro-4,4-dimethyl-3,4-dihydro-1H-quinolin-2-one

6-[3-(4-Benzo[d]isothiazol-3-yl-piperazin-1-yl)-propyl]-7-fluoro4,4-dimethyl-3,4-dihydro-1H-quinolin-2-one was prepared according to the general procedure outlined in Example 49C, starting with anhydrous potassium carbonate (1.20 g), 6-(3-chloro-propyl)-7-fluoro-4,4-dimethyl-3,4-dihydro-1H-quinolin-2-one (0.384 g, 1.42 mmol) and 3-piperazin-1-yl-benzo[d]isothiazole hydrochloride (0.63 g, 2.87 mmol). The solid was purified using an ISCO autocolumn eluting with 80% ethyl acetate in hexanes to afford 0.365 g of 6-[3-(4-Benzo[d]isothiazol-3-yl-piperazin-1-yl)-propyl]-7-fluoro-4,4-dimethyl-3,4-dihydro-1H-quinolin-2-one. 100% purity @ 254 nm; LCMS (APCI) 453.1 [M+H]⁺.

### Example 67

### 6-[3-(4-BENZO[D]ISOTHIAZOL-3-YL-PIPERAZIN-1-YL)-PROPYL]-7-FLUORO-1,4,4-TRIMETHYL-3,4-DIHYDRO-1H-QUINOLIN-2-ONE

A. 6-(3-Chloro-propyl)-7-fluoro-1,4,4-trimethyl-3,4-dihydro-1H-qu inolin-2-one

6-(3-Chloro-propyl)-7-fluoro-4,4-dimethyl-3,4-dihydro-1H-quinolin-2-one (Example 66B, 0.768 g, 2.85 mmol) was added to a stirring suspension of NaH (60% dispersion in oil, 0.137 g, 3.43 mmol), under N₂ in THF at 0° C. and stirred for 1 hr. Methyl iodide (0.62 mL, 9.96 mmol) was added dropwise at 0° C. Warmed to rt and stirred overnight. The reaction mixture was quenched with water, extracted with ethyl acetate (3×50 mL) and washed with brine. The organic extracts were dried (Na₂SO₄) and concentrated. The solid was dried in-vacuo to afford 6-(3-chloro-propyl)-7-fluoro-1,4,4-trimethyl-3,4-dihydro-1H-quinolin-2-one (0.7266 g) was afforded in 90% yield. MS (APCI): 284.1 [M+H]⁺.

B. -[3-(4-Benzo[d]isothiazol-3-yl-piperazin-1-yl)-propyl]-7-fluoro-1,4,4-trimethyl-3,4-dihydro-1H-guinolin-2-one

6-[3-(4-Benzo[d]isothiazol-3-yl-piperazin-1-yl)-propyl]-7-fluoro-1,4,4-trimethyl-3,4-dihydro-1H-quinolin-2-one was prepared according to the general procedure outlined in Example 49C, starting with anhydrous potassium carbonate (1.06 g), 6-(3-chloro-propyl)-7-fluoro-1,4,4-trimethyl-3,4-dihydro-1H-quinolin-2-one (0.7266 g, 2.56 mmol) and 3-piperazin-1-yl-benzo[d]isothiazole hydrochloride (0.842 g, 3.84 mmol). The solid was purified using an ISCO autocolumn eluting with 80% ethyl acetate in hexanes to afford 0.120 g of 6-[3-(4-benzo[d]isothiazol-3-yl-piperazin-1-yl)-propyl]-7-fluoro-1,4,4-trimethyl-3,4-dihydro-1H-quinolin-2-one. The mesylate salt was prepared by dissolving the solid in THF and MeOH and adding 1 eq. of methanesulfonic acid. The precipitate was filtered off and washed with ether to afford 6-[3-(4-benzo[d]isothiazol-3-yl-piperazin-1-yl)-propyl]-7-fluoro-1,4,4-trimethyl-3,4-dihydro-1H-quinolin-2-one mesylate. 100% purity @ 254 nm; LCMS (APCI) 467.2 [M+H]⁺.

### Example 68

### 1-{6-[3-(4-BENZO[D]ISOTHIAZOL-3-YL-PIPERAZIN-1-YL)-PROPYL]-7-FLUORO-4,4-DIMETHYL-3,4-DIHYDRO-2H-QUINOLIN-1YL}-ETHANONE

A. 6-(3-Chloro-propyl)-7-fluoro-4,4-dimethyl-1,2,3,4-tetrahydro-quinoline

To a stirring solution of 6-(3-chloro-propyl)-7-fluoro-4,4-dimethyl-3,4-dihydro-1H-quinolin-2-one (Example 66B, 0.768 g, 2.85 mmol) in THF (20 mL) at 0° C. was added BH₃.THF (1M, 38 mL) slowly via addition funnel. Warmed to room temperature and stirred overnight. The reaction mixture was quenched with aq. Na₂CO₃ and stirred for 4 hours. The precipitate was filtered off and the filtrate was extracted with ethyl acetate (3×100 mL) and washed with water and sat. NaCl. The organic extracts were dried (Na₂SO₄) and concentrated. The resultant solid was dried in-vacuo to afford 6-(3-chloro-propyl)-7-fluoro-4,4-dimethyl-1,2,3,4-tetrahydro-quinoline (0.8319 g). MS (APCI): 265.1 [M+H]⁺.

B. 1-[6-(3-Chloro-propyl)-7-fluoro-4,4-dimethyl-3,4-dihydro-2H-quinolin-1-yl]-ethanone

To a stirring solution of 6-(3-chloro-propyl)-7-fluoro-4,4-dimethyl-1,2,3,4-tetrahydro-quinoline (0.400 g, 1.56 mmol) in THF (3 mL) was added acetic acid (0.30 mL, 3.18 mmol) and triethylamine (0.30 mL). Heated to reflux and stirred overnight. The reaction mixture was quenched with water. Extracted with ethyl acetate (3×50 mL) and washed with sat. NaCl. The organic extracts were dried (Na₂SO₄) and concentrated. The resultant solid was dried in-vacuo to afford 1-[6-(3-chloro-propyl)-7-fluoro-4,4-dimethyl-3,4-dihydro-2H-quinolin-1-yl]-ethanone (0.376 g). MS (APCI): 298.1 [M+H]⁺.

C. 1-{6-[3-(4-Benzo[d]isothiazol-3-yl-piperazin-1-yl)-propyl]-7-fluoro-4,4-dimethyl-3,4-dihydro-2H-quinolin-1 yl}-ethanone

1-{6-[3-(4-Benzo[d]isothiazol-3-yl-piperazin-1-yl)-propyl]-7-fluoro-4,4-dimethyl-3,4-dihydro-2H-quinolin-1yl}-ethanone was prepared according to the general procedure outlined in Example 49C, starting with anhydrous potassium carbonate (0.699 g), 1-[6-(3-chloro-propyl)-7-fluoro-4,4-dimethyl-3,4-dihydro-2H-quinolin-1-yl]-ethanone (0.376 g, 1.26 mmol) and 3-piperazin-1-yl-benzo[d]isothiazole hydrochloride (0.554 g, 2.53 mmol). The solid was purified using an ISCO autocolumn eluting with 80% ethyl acetate in hexanes to afford 0.300 g of 1-{6-[3-(4-benzo[d]isothiazol-3-yl-piperazin-1-yl)-propyl]-7-fluoro-4,4-dimethyl-3,4-dihydro-2H-quinolin-1yl}-ethanone. 100% purity @ 254 nm; LCMS (APCI) 481.2 [M+H]⁺. CHN: calculated for C₂₇H₃₃F₁N₄O₁S₁ C: 67.47%, H: 6.92, N: 11.66%; found, C: 67.18%, H: 6.98%, N: 11.48%.

### Example 69

### 1-{6-[3-(4-BENZO[D]ISOTHIAZOL-3-YL-PIPERAZIN-1-YL)-PROPYL]-4,4-DIMETHYL-3,4-DIHYDRO-2H-QUINOLIN-1YL}-ETHANONE

A. 6-(3-Chloro-Propyl)-4,4-dimethyl-1,2,3,4-tetrahydro-quinoline

6-(3-Chloro-propyl)-4,4-dimethyl-1,2,3,4-tetrahydro-quinoline was prepared according to the general procedure outlined in step A of Example 68, starting with 6-(3-chloro-propyl)-4,4-dimethyl-3,4-dihydro-1H-quinolin-2-one (Example 49B, 1.50 g, 5.96 mmol), BH₃.THF (1M, 30 mL) and THF (25 mL). The solid was dried in-vacuo to afford 6-(3-Chloro-propyl)-4,4-dimethyl-1,2,3,4-tetrahydro-quinoline (0.520). MS (APCI): [M+H]+238.1.

B. 1-[6-(3-Chloro-propyl)-4,4-dimethyl-3,4-dihydro-2H-quinolin-1-yl]-ethanone

1-[6-(3-chloro-propyl)-4,4-dimethyl-3,4-dihydro-2H-quinolin-1-yl]-ethanone was prepared according to the general procedure outlined in step B of Example 68, starting with 6-(3-chloro-propyl)-4,4-dimethyl-1,2,3,4-tetrahydro-quinoline (0.500 g, 2.10 mmol), acetic acid (0.39 mL, 4.13 mmol) and triethylamine (0.39 mL). The solid was purified using an ISCO autocolumn eluting with 1:1 dichlormethane/ethyl acetate, 2% MeOH and dried in-vacuo to afford 1-[6-(3-chloro-propyl)-4,4-dimethyl-3,4-dihydro-2H-quinolin-1-yl]-ethanone (0.390 g). MS (APCI): 280.1 [M+H]⁺.

C. 1-{6-[3-(4-Benzo[d]isothiazol-3-yl-piPerazin-1-yl)-propyl]-4,4-dimethyl-3,4-dihydro-2H-quinolin-1yl}-ethanone

1-{6-[3-(4-Benzo[d]isothiazol-3-yl-piperazin-1-yl)-propyl]-4,4-dimethyl-3,4-dihydro-2H-quinolin-1yl}-ethanone was prepared according to the general procedure outlined in Example 49C, starting with anhydrous potassium carbonate (0.77 g), 1-[6-(3-chloro-propyl)-4,4-dimethyl-3,4-dihydro-2H-quinolin-1-yl]-ethanone (0.390 g, 1.39 mmol) and 3-piperazin-1-yl-benzo[d]isothiazole hydrochloride (0.610 g, 2.79 mmol). The solid was purified using an ISCO autocolumn eluting with 80% ethyl acetate in hexanes to afford 0.108 g of 1-{6-[3-(4-benzo[d]isothiazol-3-yl-piperazin-1-yl)-propyl]-4,4-dimethyl-3,4-dihydro-2H-quinolin-1yl}-ethanone. 100% purity @ 254 nm; LCMS (APCI) 463.2 [M+H]⁺.

### Example 70

### 1-{6-[3-(4-BENZO[D]ISOTHIAZOL-3-YL-PIPERAZIN-1-YL)-PROPYL]-3,3-DIMETHYL-3,4-DIHYDRO-2H-QUINOLIN-1YL}-ETHANONE

A. 6-(3-Chloro-propyl)-3,3-dimethyl-1,2,3,4,-tetrahydro-quinoline

6-(3-Chloro-propyl)-3,3-dimethyl-1,2,3,4-tetrahydro-quinoline was prepared according to the general procedure outlined in step A of Example 68, starting with 6-(3-chloro-propyl)-3,3-dimethyl-3,4-dihydro-1H-quinolin-2-one (1.70 g, 6.77 mmol), BH₃THF (1M, 30 mL) and THF (20 mL). The olid was dried in-vacuo to afford 6-(3-Chloro-propyl)-3,3-dimethyl-1,2,3,4-tetrahydro-quinoline (1.60 g). MS (APCI): [M+H]⁺238.1.

B. 1-[6-(3-chloro-propyl)-3,3-dimethyl-3,4-dihydro-2H-quinolin-1-yl]-ethanone

1-[6-(3-chloro-propyl)-3,3-dimethyl-3,4-dihydro-2H-quinolin-1-yl]-ethanone was prepared according to the general procedure outlined in step B of Example 68, starting with 6-(3-chloro-propyl)-3,3-dimethyl-1,2,3,4-tetrahydro-quinoline (1.0 g, 4.21 mmol), acetic acid (0.794 mL, 8.41 mmol) and triethylamine (0.794 mL). The solid was purified using an ISCO autocolumn eluting with 4:1 ethyl acetate/hexanes and dried in-vacuo to afford 1-[6-(3-chloro-propyl)-3,3-dimethyl-3,4-dihydro-2H-quinolin-1-yl]-ethanone (0.8002 g). MS (APCI): 280.1 [M+H]⁺.

C. 1-{6-[3-(4-Benzo[d]isothiazol-3-yl-Piperazin-1-yl)-propyl]-3,3-dimethyl-3,4-dihydro-2H-quinolin-1yl}-ethanone

1-{6-[3-(4-Benzo[d]isothiazol-3-yl-piperazin-1-yl)-propyl]-3,3-dimethyl-3,4-dihydro-2H-quinolin-1yl}-ethanone was prepared according to the general procedure outlined in Example 49C, starting with anhydrous potassium carbonate (1.52 g), 1-[6-(3-chloro-propyl)-3,3-dimethyl-3,4-dihydro-2H-quinolin-1-yl]-ethanone (0.80 g, 2.86 mmol) and 3-piperazin-1-yl-benzo[d]isothiazole hydrochloride (1.0 g, 4.56 mmol). The solid was purified using an ISCO autocolumn eluting with 80% ethyl acetate in hexanes to afford 0.250 g of 1-{6-[3-(4-benzo[d]isothiazol-3-yl-piperazin-1-yl)-propyl]-3,3-dimethyl-3,4-dihydro-2H-quinolin-1yl}-ethanone. The mesylate salt was prepared by dissolving the solid in THF and MeOH and adding 1 eq. of methanesulfonic acid. The precipitate was filtered off and washed with ether to afford 1-{6-[3-(4-benzo[d]isothiazol-3-yl-piperazin-1-yl)-propyl]-3,3-dimethyl-3,4-dihydro-2H-quinolin-1yl}-ethanone mesylate. 100% purity @ 254 nm; LCMS (APCI) 463.2 [M+H]⁺.

### Example 71

### 6-[3-(4-BENZO[D]ISOTHIAZOL-3-YL-PIPERAZIN-1-YL)-PROPYL]-1,3,3-TRIMETHYL-1,2,3,4-TETRAHYRDO-QUINOLINE

A. 6-(3-Chloro-propyl)-1,3,3-trimethyl-1,2,3,4-tetrahydro-quinoline

6-(3-Chloro-propyl)-1,3,3-trimethyl-1,2,3,4-tetrahydro-quinoline was prepared according to step A of Example 67, starting with 6-(3-chloro-propyl)-3,3-dimethyl-3,4-dihydro-1H-quinolin-2-one (0.482 g, 2.03 mmol), NaH (60% dispersion in oil, 0.106 g, 2.65 mmol) and methyl iodide (0.510 mL, 8.19 mmol. The solid was dried in-vacuo to afford 6-(3-chloro-propyl)-1,3,3-trimethyl-1,2,3,4-tetrahydro-quinoline (0.165 g). MS (APCI): 252.1 [M+H]⁺.

B. 6-[3-(4-Benzo[d]isothiazol-3-yl-piperazin-1-yl)-propyl]-1,3,3-trimethyl-1,2,3,4-tetrahyrdo-quinoline

6-[3-(4-Benzo[d]isothiazol-3-yl-piperazin-1-yl)-propyl]-1,3,3-trimethyl-1,2,3,4-tetrahyrdo-quinoline was prepared according to the general procedure outlined in Example 49C, starting with anhydrous potassium carbonate (0.209 9), 6-(3-chloro-propyl)-1,3,3-trimethyl-1,2,3,4-tetrahydro-quinoline (0.1651 g, 0.656 mmol) and 3-piperazin-1-yl-benzo[d]isothiazole hydrochloride (0.230 g, 1.05 mmol). The solid was purified using an ISCO autocolumn eluting with 80% ethyl acetate in hexanes to afford 0.093 g of 6-[3-(4-benzo[d]isothiazol-3-yl-piperazin-1-yl)-propyl]-1,3,3-trimethyl-1,2,3,4-tetrahyrdo-quinoline. The mesylate salt was prepared by dissolving the solid in THF and MeOH and adding 1 eq. of methanesulfonic acid. The precipitate was filtered off and washed with ether to afford 6-[3-(4-benzo[d]isothiazol-3-yl-piperazin-1-yl)-propyl]-1,3,3-trimethyl-1,2,3,4-tetrahyrdo-quinoline mesylate. 100% purity @ 254 nm; LCMS (APCI) 435.1 [M+H]⁺. ¹H NMR (400 MHz, CDCl₃) δ

### Example 72

### 6-[3-(4-BENZO[D]ISOTHIAZOL-3-YL-PIPERAZIN-1-YL)-PROPYL]-8-CHLORO-4,4-DIMETHYL-3,4-DIHYDRO-1H-QUINOLIN-2-ONE

A. 6-(3-Chloro-propionyl)-8-chloro-4,4-dimethyl-3,4-dihydro-1H-quinolin-2-one

6-(3-Chloro-propionyl)-8-chloro-4,4-dimethyl-3,4-dihydro-1H-quinolin-2-one was prepared according to the general procedure outlined in step C of example 27, starting with 8-Chloro-4,4-dimethyl-3,4-dihydro-1H-quinolin-2-one (2.0 g, 9.54 mmol), aluminum chloride (11.0 g, 82.5 mmol) and chloropropionyl chloride (2.97 mL, 35.8 mmol). The precipitate was filtered off and washed with ample amounts of water and dried in-vacuo to afford 6-(3-chloro-propionyl)-8-chloro-4,4-dimethyl-3,4-dihydro-1H-quinolin-2-one (0.439 g). MS (APCI): 300.0 [M+H]⁺.

B. 6-(3-Chloro-propyl)-8-chloro-4,4-dimethyl-3,4-dihydro-1H-quinolin-2-one

6-(3-Chloro-propyl)-8-chloro-4,4-dimethyl-3,4-dihydro-1H-quinolin-2-one was prepared according to the general procedure outlined in step D of Example 27, starting with 6-(3-chloro-propyl)-8-chloro-4,4-dimethyl-3,4-dihydro-1H-quinolin-2-one (0.439 g, 1.46 mmol), trifluoroacetic acid (0.676 mL, 8.77 mmol) and triethylsilane (0.584 mL, 3.66 mmol). The precipitate was filtered off, washed with ample amounts of water, and dried in-vacuo to afford 8-chloro-6-(3-chloro-propyl)-4,4-dimethyl-3,4-dihydro-1H-quinolin-2-one (0.417 g). MS (APCI): 286.0 [M+H]⁺.

C. 6-[3-(4-Benzo[d]isothiazol-3-yl-piperazin-1-yl)-propyl]-8-chloro-4,4-dimethyl-3,4-dihydro-1H-quinolin-2-one

6-[3-(4-Benzo[d]isothiazol-3-yl-piperazin-1-yl)-propyl]-8-chloro-4,4-dimethyl-3,4-dihydro-1H-quinolin-2-one was prepared according to the general procedure outline in step C of Example 49, starting with anhydrous potassium carbonate (0.200 g), 6-(3-chloro-propyl)-8-chloro-4,4-dimethyl-3,4-dihydro-1H-quinolin-2-one (0.375 g, 1.31 mmol), and 3-piperazin-1-yl-benzo[d]isothiazole hydrochloride (0.300 g, 1.37 mmol). The solid was purified using an ISCO autocolumn eluting with 4:1 ethyl acetate/hexanes and dried in-vacuo to afford 6-[3-(4-benzo[d]isothiazol-3-yl-piperazin-1-yl)-propyl]-8-chloro-4,4-dimethyl-3,4-dihydro-1H-quinolin-2-one (0.080 g). MS (APCI): 468.2 [M+H]⁺.

### Example 73

### 6-[3-(4-BENZO[D]ISOXAZOL-3-YL-PIPERAZIN-1-YL)-P ROPYL]-8-CHLORO-4,4-DIMETHYL-3,4-DIHYDRO-1H-QUINOLIN-2-ONE

6-[3-(4-Benzo[d]isoxazol-3-yl-piperazin-1-yl)-propyl]-8-chloro-4,4-dimethyl-3,4-dihydro-1H-quinolin-2-one was prepared according to the general procedure outlined in Example 49C, starting with anhydrous potassium carbonate (1.5 g, 29.6 mmol), 6-(3-chloro-propyl)-8-chloro-4,4-dimethyl-3,4-dihydro-1H-quinolin-2-one (0.375 g, 1.31 mmol), and 3-piperazin-1-yl-benzo[d]isoxazole (0.63 g, 2.63 mmol). The solid was purified using an ISCO autocolumn eluting with 4:1 ethyl acetate/hexanes and dried in-vacuo to afford 6-[3-(4-benzo[d]isoxazol-3-yl-piperazin-1-yl)-propyl]-8-chloro-4,4-dimethyl-3,4-dihydro-1H-quinolin-2-one (0.100 g). MS (APCI): 453.2 [M+H]⁺.

### Example 74

### 6-[3-(4-BENZO[D]ISOTHIAZOL-3-YL-PIPERAZIN-1-YL)-PROPYL]-4,4,8-TRIMETHYL-3,4-DIHYDRO-1H-QUINOLIN-2-ONE

A. 6-(3-Chloro-propionyl)-4,4,8-trimethyl-3,4-dihydro-1H-quinolin-2-one

6-(3-Chloro-propionyl)-4,4,8-trimethyl-3,4-dihydro-1H-quinolin-2-one was prepared according to the general procedure outlined in step A of Example 49, starting with 4,4,8-trimethyl-3,4-dihydro-1H-quinolin-2-one (1.0 g, 5.28 mmol), aluminum chloride (2.82 g, 21.5 mmol) and chloropropionyl chloride (0.526 mL, 6.34 mmol). The resultant precipitate was filtered off and washed with ample amounts of water and dried in-vacuo to afford 6-(3-chloro-propionyl)-4,4,8-trimethyl-3,4-dihydro-1H-quinolin-2-one (1.27 g). MS (APCI): 280.1 [M+H]⁺.

B. 6-(3-Chloro-propyl)-4,4,8-trimethyl-3,4-dihydro-1H-quinolin-2-one

6-(3-Chloro-propyl)-4,4,8-trimethyl-3,4-dihydro-1H-quinolin-2-one was prepared according to the general procedure outlined in step B of Example 49, starting with 6-(3-chloro-propionyl)-4,4,8-trimethyl-3,4-dihydro-1H-quinolin-2-one (1.27 g, 4.54 mmol), trifluoroacetic acid (2.1 mL, 27.3 mmol) and triethylsilane (1.81 mL, 11.3 mmol. The resultant precipitate was filtered off, washed with ample amounts of water, and dried in-vacuo to afford 6-(3-chloro-propyl)-4,4,8-trimethyl-3,4-dihydro-1H-quinolin-2-one (0.693 g). MS (APCI): 266.1 [M+H]⁺.

C. 6-[3-(4-Benzo[d]isothiazol-3-yl-piperazin-1-yl)-propyl]-4,4,8-trimethyl-3,4-dihydro-1H-quinolin-2-one

6-[3-(4-Benzo[d]isothiazol-3-yl-piperazin-1-yl)-propyl]-4,4,8-trimethyl-3,4-dihydro-1H-quinolin-2-one was prepared according to the general procedure outlined in step C of Example 49, starting with anhydrous potassium carbonate (0.375 g), 6-(3-chloro-propyl)-4,4,8-trimethyl-3,4-dihydro-1H-quinolin-2-one (0.30 g, 1.1 mmol) and 3-piperazin-1-yl-benzo[d]isothiazole hydrochloride (0.297 g, 1.35 mmol). The solid was purified using an ISCO autocolumn eluting with 4:1 ethyl acetate/hexanes and dried in-vacuo to afford 6-[3-(4-benzo[d]isothiazol-3-yl-piperazin-1-yl)-propyl]-4,4,8-trimethyl-3,4-dihydro-1H-quinolin-2-one (0.0896 g). MS (APCI): 449.2 [M+H]⁺.

### Example 75

### 6-[3-(4-BENZO[D]ISOXAZOL-3-YL-PIPERAZIN-1-YL)-PROPYL]-4,4,8-TRIMETHYL-3,4-DIHYDRO-1H-QUINOLIN-2-ONE

6-[3-(4-Benzo[d]isoxazol-3-yl-piperazin-1-yl)-propyl]-4,4,8-trimethyl-3,4-dihydro-1H-quinolin-2-one was prepared according to the general procedure outlined in Example 49C, starting with anhydrous potassium carbonate (1.56 g, 11.3 mmol), 6-(3-chloro-propyl)-4,4,8-trimethyl-3,4-dihydro-1H-quinolin-2-one (0.30 g, 1.1 mmol), and 3-piperazin-1-yl-benzo[d]isoxazole (0.54 g, 2.26 mmol). The solid was purified using an ISCO autocolumn eluting with 4:1 ethyl acetate/hexanes and dried in-vacuo to afford 6-[3-(4-benzo[d]isoxazol-3-yl-piperazin-1-yl)-propyl]-4,4,8-trimethyl-3,4-dihydro-1H-quinolin-2-one (0.257 g). MS (APCI): 433.2 [M+H]⁺.

### Example 76

### 6-[3-(4-BENZO[D]ISOTHIAZOL-3-YL-PIPERAZIN-1-YL)-PROPYL]-8-ETHYL-4,4-DIMETHYL-3,4-DIHYDRO-1H-QUINOLIN-2-ONE

A. 6-(3-Chloro-propionyl)-8-ethyl-4,4-dimethyl-3,4-dihydro-1H-quinolin-2-one

6-(3-Chloro-propionyl)-8-ethyl-4,4-dimethyl-3,4-dihydro-1H-quinolin-2-one was prepared according to the general procedure outlined in step A of example 49, starting with 8-ethyl-4,4-dimethyl-3,4-dihydro-1H-quinolin-2-one (2.0 g, 9.84 mmol), aluminum chloride (5.25 g, 39.37 mmol) and chloropropionyl chloride (0.98 mL, 11.81 mmol). The precipitate was filtered off and washed with ample amounts of water and dried in-vacuo to afford 6-(3-chloro-propionyl)-8-ethyl-4,4-dimethyl-3,4-dihydro-1H-quinolin-2-one (2.86 g). MS (APCI): 294.1 [M+H]⁺.

B. 6-(3-Chloro-propyl)-8-ethyl-4,4-dimethyl-3,4-dihydro-1H-quinolin-2-one

6-(3-Chloro-propyl)-8-ethyl-4,4-dimethyl-3,4-dihydro-1H-quinolin-2-one was prepared according to the general procedure outlined in step B of Example 49, starting with 6-(3-chloro-propionyl)-8-ethyl-4,4-dimethyl-3,4-dihydro-1H-quinolin-2-one (2.86 g, 9.74 mL), trifluoroacetic acid (4.5 mL, 58.4 mmol) and triethylsilane (3.89 mL, 24.4 mmol). The precipitate was filtered off, washed with ample amounts of water, and dried in-vacuo to afford 6-(3-chloro-propyl)-8-ethyl-4,4-dimethyl-3,4-dihydro-1H-quinolin-2-one (1.66 g). MS (APCI): 280.1 (M+H]⁺.

C. 6-[3-(4-Benzo[d]isothiazol-3-yl-piperazin-1-yl)-propyl]-8-ethyl-4,4-dimethyl-3,4-dihydro-1H-quinolin-2-one

6-[3-(4-Benzo[d]isothiazol-3-yl-piperazin-1-yl)-propyl]-8-ethyl-4,4-dimethyl-3,4-dihydro-1H-quinolin-2-one was prepared according to the general procedure outline in step C of Example 49, starting with anhydrous potassium carbonate (1.30 g), 6-(3-chloro-propyl)-8-ethyl-4,4-dimethyl-3,4-dihydro-1H-quinolin-2-one (0.70 g, 2.5 mmol), and 3-piperazin-1-yl-benzo[d]isothiazole hydrochloride (0.823 g, 3.75 mmol). The solid was purified using an ISCO autocolumn eluting with 4:1 ethyl acetate/hexanes and dried in-vacuo to afford 6-[3-(4-benzo[d]isothiazol-3-yl-piperazin-1-yl)-propyl]-8-ethyl-4,4-dimethyl-3,4-dihydro-1H-quinolin-2-one (0.223 g). MS (APCI): 463.2 [M+H]⁺.

### Example 77

### 6-[3-(4-BENZO[D]ISOXAZOL-3-YL-PIPERAZIN-1-YL)-PROPYL]-8-EHTYL-4,4-DIMETHYL-3,4-DIHYDRO-1H-QUINOLIN-2-ONE

6-[3-(4-Benzo[d]isoxazol-3-yl-piperazin-1-yl)-propyl]-8-ethyl-4,4-dimethyl-3,4-dihydro-1H-quinolin-2-one was prepared according to the general procedure outlined in Example 49C, starting with anhydrous potassium carbonate (4.1 9, 29.6 mmol), 6-(3-chloro-propyl)-8-ethyl-4,4-dimethyl-3,4-dihydro-1H-quinolin-2-one (0.700 g, 2.5 mmol), and 3-piperazin-1-yl-benzo[d]isoxazole (1.19 9, 4.96 mmol). The solid was purified using an ISCO autocolumn eluting with 4:1 ethyl acetate/hexanes and dried in-vacuo to afford 6-[3-(4-benzo[d]isoxazol-3-yl-piperazin-1-yl)-propyl]-8-ethyl-4,4-dimethyl-3,4-dihydro-1H-quinolin-2-one (0.4225 g). MS (APCI): 447.2 [M+H]⁺.

Examples 78 through 87 were prepared according to the following synthetic route:

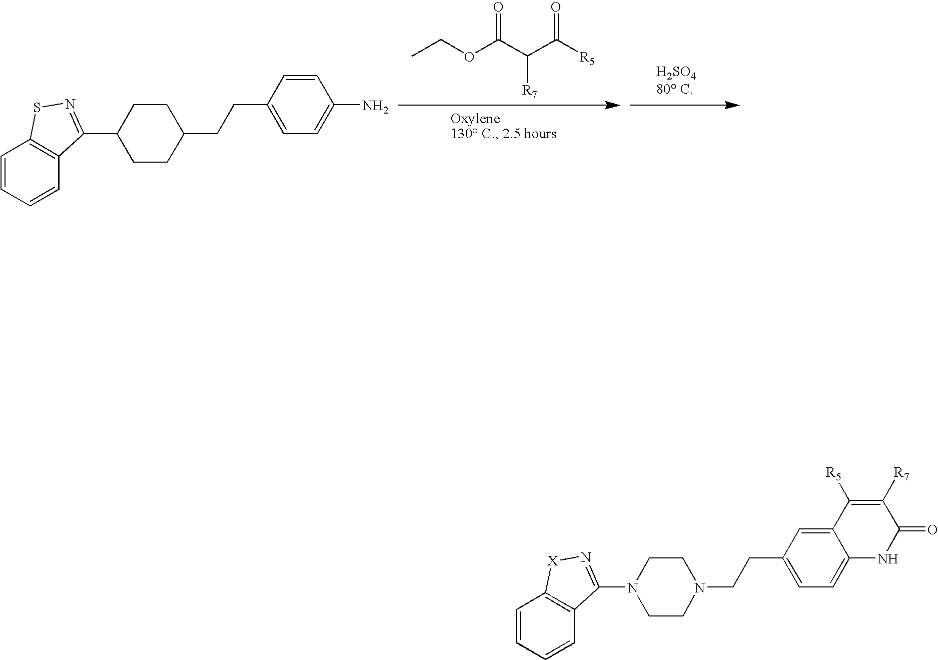

### Example 78

### 6-[2-(4-BENZO[D]ISOTHIAZOL-3-YL-PIPERAZIN-1-YL)-ETHYL]-4-METHYL-1H-QUINOLIN-2-ONE

In an open tube (8 ml) equipped with a stir bar, the aniline (IP 901A, 1.0 mmol, 338 mgs), o-xylene (1 ml) and ethyl acetoacetate (1.1 mmol, 140 ul) were combined. The mixture was then warmed to 130° C. in an aluminum heating block for 2½ hours. (TLC and MS showed only a trace of remaining aniline). Reaction was cooled and concentrated to dryness (light yellow oil). The crude amide was then treated with 1 ml of sulfuric acid and reaction was sealed and warmed to 80° C. for 1 hours. The reaction was cooled and poured into water/ice. The pH was brought to neutral(˜7) with 50% NaOH. The precipitate was filtered and dried to constant weight. The crude was then dissolved in 400:8:1 (CH₂Cl₂:EtOH:NH₄OH) and loaded onto a silica gel cartridge and purified via MPLC, (silica cartridge, 40 g) eluting with gradient of methylene chloride to (100:8:1) methylene chloride:Ethanol:Ammonia Hydroxide over a 1 hour period, yielding pure product (193 mgs, 47.7% yield). Crystals were triturated with Acetonitrile and filtered. MS (APCI): 405 [M+H]. 1H NMR (400 MHz, DMSO-D6) δ ppm 2.39 (d, J=1.22 Hz, 3H) 2.63 (m, 6H) 2.81 (m, 2H) 3.30 (d, J=9.52 Hz, 8H) 3.42 (m, 4H) 6.34 (s, 1H) 7.20 (d, J=8.30 Hz, 1H) 7.39 (m, 2H) 7.53 (m, 2H) 8.02 (d, J=8.30 Hz, 2H) 11.50 (s,1H).

The title compound of Examples 79 through 87 were prepared using a procedure analogous to that of Example 78.

### Example 79

### 6-[2-(4-BENZO[D]ISOTHIAZOL-3-YL-PIPERAZIN-1-YL)-ETHYL]-3,4-DIMETHYL-1H-QUINOLIN-2-ONE

MS (APCI): 419 [M+H].

### Example 80

### 6-[2-(4-BENZO[D]ISOTHIAZOL-3-YL-PIPERAZIN-1-YL)-ETHYL]-4-ETHYL-1H-QUINOLIN-2-ONE

MS (APCI): 419 [M+H].

### Example 81

### 8-[2-(4-BENZO[D]ISOTHIAZOL-3-YL-PIPERAZIN-1-YL)-ETHYL]-1,2,3,5-TETRAHYDRO-CYCLOPENTA[C]QUINOLIN-4-ONE

MS (APCI): 431 [M+H].

### Example 82

### 6-[2-(4-BENZO[D]ISOTHIAZOL-3-YL-PIPERAZIN-1-YL)-ETHYL]-3-ETHYL-4-METHYL-1H-QUINOLIN-2-ONE

MS (APCI): 433 [M+H].

### Example 83

### 6-[2-(4-BENZO[D]ISOTHIAZOL-3-YL-PIPERAZIN-1-YL)-ETHYL]-4-PROPYL-1H-QUINOLIN-2-ONE

MS (APCI): 433 [M+H].

### Example 84

### 6-[2-(4-BENZO[D]ISOTHIAZOL-3-YL-PIPERAZIN-1-YL)-ETHYL]-4-ISOPROPYL-1H-QUINOLIN-2-ONE

MS (APCI): 433 [M+H].

### Example 85

### 2-[2-(4-BENZO[D]ISOTHIAZOL-3-YL-PIPERAZIN-1-YL)-ETHYL]-7,8,9,10-TETRAHYDRO-5H-PHENANTHRIDIN-6-ONE

MS (APCI): 445 [M+H].

### Example 86

### 6-[2-(4-BENZO[D]ISOTHIAZOL-3-YL-PIPERAZIN-1-YL)-ETHYL]-4-TRIFLUOROMETHYL-1H-QUINOLIN-2-ONE

MS (APCI): 459 [M+H].

### Example 87

### 6-[2-(4-BENZO[D]ISOTHIAZOL-3-YL-PIPERAZIN-1-YL)-ETHYL]-4-PHENYL-1H-QUINOLIN-2-ONE

MS (APCI): 467 [M+H].

### Example 88

### 9-[2-(4-BENZO[D]ISOTHIAZOL-3-YL-PIPERAZIN-1-YL)-ETHYL]-1,2,6,7-TETRAHYDRO-5H-PYRIDO[3,2,1-IJ]QUINOLIN-3-ONE

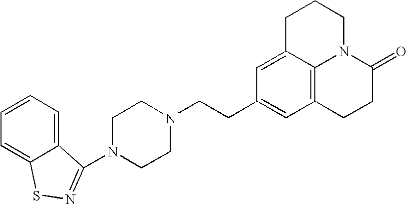

A. 9-(2-Chloroacetyl)-1,2,6,7-tetrahydro-5H-pyrido[3,2,1-ij]quinolin-3-one

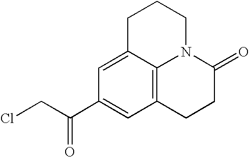

Chloroacetyl chloride (1.56 ml, 19.6 mmol) was added to a mixture of 1,2,6,7-tetrahydro-5H-pyrido[3,2,1-ij]quinolin-3-one (2.04 g, 10.9 mmol, Tetrahedron, 1986, 42, 5407) and aluminum chloride (7.27 g, 54.5 mmol) in carbon disultide (50 ml) with vigorous stirring. The reaction mixture was heated at reflux for 2 hours and cooled to room temperature. The solvent was decanted and the residue was slowly treated with cold water under vigorous agitation. After quenching, the gold, amorphous solid was collected, washed with water, and dried. Yield=2.51 g (87%); MS(APCI), (M+1)⁺=264, (M−1)⁺=262.

B. 9-(2-Chloroethyl)-1,2,6.7-tetrahydro-5H-pyrido[3,2,1-ij]quinolin-3-one

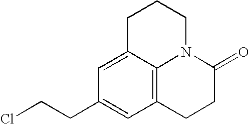

The product from Example 88A (2.51 g, 9.52 mmol) was added to 7.30 ml (95.2 mmol) trifluoroacetic acid and the stirred mixture was cooled to 0° C. under an atmosphere of nitrogen. Triethylsilane (3.52 ml, 21.8 mmol) was added portionwise and the reaction mixture was heated at 45° C. for 20 minutes and kept at room temperature for 15 hours. The reaction mixture was poured into water and extracted with ethyl acetate. The organic extract was dried over magnesium sulfate, filtered, and concentrated. Elution through a flash column (silica gel 60, 230-400 mesh, 1:1 hexanes : EtOAc) gave a yellow oil which crystallized on standing. Yield=1.90 g (80%); MS(APCI), (M+1)⁺=250, (M+3)⁺=253.

C. 9-[2-(4-Benzo[d]isothiazol-3-yl-piperazin-1-yl)-ethyl]-1,2,6,7-tetrahydro-5H-pyrido [3,2,1-ij]quinolin-3-one

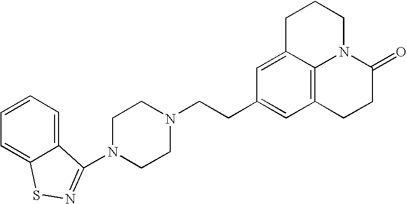

A mixture of 3-piperazin-1-yl-benzo[d]isothiazole hydrochloride (1.62 g, 6.34 mmol), product from Example 88B (1.90 g, 7.61 mmol), anhydrous potassium carbonate (1.93 g, 13.9 mmol), and potassium iodide (200 mg) in acetonitrile (80 ml) was refluxed for 48 hours. The reaction mixture was concentrated and the residue was partitioned between methylene chloride and water. The organic layer was dried over magnesium sulfate, filtered, and concentrated. The crude product was eluted through a flash column (silica gel 60, 230-400 mesh, EtOAc) to give a clear, viscous oil which crystallized on standing. Yield=1.16 g (42%); MS(APCI), (M+1)⁺=433. ¹H-NMR (DMSO-d₆, δ) 8.03 (d, 2H, J=9.0 Hz), 7.53 (t, 1H, J=8.1, 7.8 Hz), 7.40 (t, 1H, J=8.1, 7.3 Hz), 6.87 (d, 2H, J=7.3 Hz), 3.69 (t, 2H, J=5.9, 5.9 Hz), 3.26-3.43 (m, 6H), 2.46-2.78 (m, 12H), 1.78 (m, 2H). CHN: calculated for C₂₅H₂₈N₄OS; C, 69.41%, H, 6.52%, N, 12.95%; found C, 69.28%, H, 6.60%, N, 12.65%.

### Example 89

### 9-[2-(4-BENZO[D]ISOTHIAZOL-3-YL-PIPERAZIN-1-YL)-ETHYL]-1,1-DIMETHYL-1,2,6,7-TETRAHYDRO-5H-PYRIDO[3,2,1-IJ]QUINOLIN-3-ONE

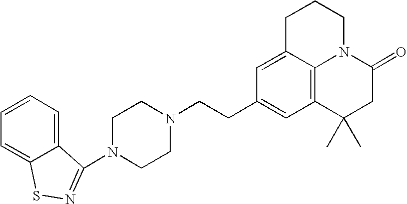

A. 1-(3,4-Dihydro-2H-quinolin-1-yl)-3-methyl-but-2-en-1-one

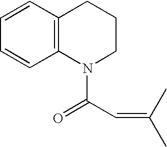

To a vigorously stirred solution of 1,2,3,4-tetrahydroquinoline (10.82 ml, 0.086 mol) in dry acetone (80 ml) was slowly added 3,3-dimethylacryloyl chloride (10 ml, 0.090 mol). The reaction mixture was refluxed for 7 hours and poured into 300 ml dilute aqueous hydrochloric acid. The aqueous mixture was extracted with chloroform and the organic extract was concentrated to a red oil. Yield=15.08 g (81%); MS (APCI), (M+1)⁺=216.

B. 1,1-Dimethyl-1,2,6,7-tetrahydro-5H-pyrido[3,2,1-ij]quinolin-3-one

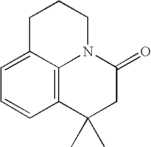

The product from Example 89A (15.08 g, 0.07 mol) was mixed with aluminum chloride (22.54 g, 0.169 mol, exotherm) and the neat mixture was heated at 90° C. until evolution of HCl ceased. Upon cooling, the reaction mixture was quenched with cold water and extracted with chloroform. The organic extract was dried over magnesium sulfate, filtered, and concentrated. The crude product was eluted through a flash column (silica gel 60, 230-400 mesh, 3: 2 hexanes: EtOAc) to give a reddish-orange oil. Yield=3.91 g (26%); MS (APCI), (M+1)⁺=216.

C. 9-(2-Chloroacetyl)-1,1-dimethyl-1,2,6,7-tetrahydro-5H-pyrido[3,2,1-ij]quinolin-3-one

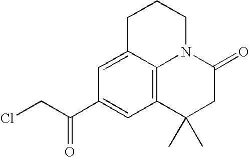

In accordance with the preparation of Example 88A, the product from Example 89B (3.91 g, 0.0182 mol) underwent Friedel-Crafts acylation with chloroacetyl chloride to give the desired product as a brown, amorphous solid. Yield=5.24 g (99%); MS (APCI), (M+1)⁺=292, (M−1)⁺=290, (M+3)⁺=294.

D. 9-(2-Chloroethyl)-1,1-dimethyl-1,2,6,7-tetrahydro-5H-pyrido[3,2,1-ij]quinolin-3-one

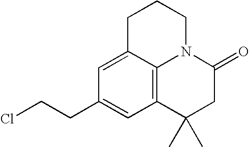

The product from Example 89C (5.24 g, 0.018 mol) underwent reduction based on the procedure for Example 88B to give the title compound as an orange oil which crystallized on standing. Yield=4.63 g (93%); MS (APCI), (M+1)⁺=278, (M+3)⁺=280.

E. 9-[2-(4-Benzo[d]isothiazol-3-yl-piperazin-1-yl)-ethyl]-1,1-dimethyl-1,2,6,7-tetrahydro-5H-pyrido3,2,1-iilquinolin-3-one

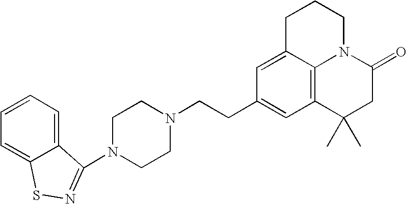

The product from Example 89D (849 mg, 3.06 mmol) was reacted with 3-piperazin-1-yl-benzo[d]isothiazole hydrochloride (652 mg, 2.55 mmol) based on the procedure described for Example 88C. The title compound was taken up in methylene chloride and the solution treated with 4.0 N HCl solution in 1,4-dioxane to precipitate the hydrochloride salt. Yield=342 mg (27%); MS (APCI), (M+1)⁺=461. ¹H-NMR (DMSO-d₆, δ): 8.11 (t, 2H, J=4.4, 8.3 Hz), 7.58 (t, 1H, J=7.3, 7.6 Hz), 7.45 (t, 1H, J=7.6, 7.1 Hz), 7.05 (s, 1H), 6.94 (s, 1H), 4.1 (d, 2H, J=12.9 Hz), 3.74-3.00 (m, 10H), 2.73-2.47 (m, 3H), 2.39 (s, 2H), 1.80 (br s, 2H), 1.19 (s, 6H). CHN: calculated for C₂₇H₃₂N₄OS 1.3 HCl, C, 63.83%, H, 6.61%, N, 11.03%; found C, 63.56%, H, 6.64%, N, 10.48%.

### Example 90

### 8-[2-(4-BENZO[D]ISOTHIAZOL-3-YL-PIPERAZIN-1-YL)-ETHYL]-6,6-DIMETHYL-1,2,5,6-TETRAHYDROPYRROLO[3,2,1-IJ]QUINOLIN-4-ONE

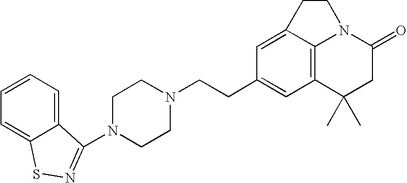

A. 6,6-Dimethyl-1,2,5,6-tetrahydropyrrolo[3,2,1-ij]quinolin-4-one

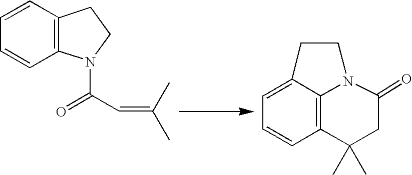

1-(2,3-Dihydroindol-1-yl)-3-methyl-but-2-en-1-one (5.0 g, 0.025 mol. Prepared from 2,3-dihydroindole and 3,3-dimethylacryloyl chloride according to Example 89A) was reacted with aluminum chloride in a manner similar to Example 89B to give the title compound as an orange solid. Yield=4.28 g (85%); MS (APCI), (M+1)⁺=202.

B. 8-(2-Chloroacetyl)-6,6-dimethyl-1,2,5,6-tetrahydropyrrolo[3,2,1-ij]quinolin-4-one

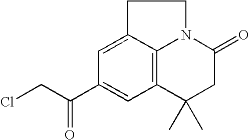

Example 90A (4.28 g, 0.021 mol) underwent Friedel-Crafts acylation based on the procedure described in Example 88A to give the title compound as a light-brown, amorphous solid. Yield=5.80 g (99%); MS (APCI), (M+1)⁺=278, (M−1)⁺=276, (M+3)⁺=280.

C. 8-(2-Chloroethyl)-6,6-dimethyl-1,2,5,6-tetrahydropyrrolo[3,2,1-ij]quinolin-4-one

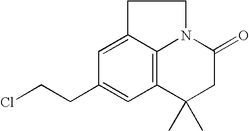

Example 90B (5.80 g, 0.021 mol) underwent reduction based on the procedure described in Example 88B to give the title compound as an orange oil which crystallized on standing. Yield=5.27 g (95%); MS (APCI), (M+1)⁺=264, (M+3)⁺=266.

D. 8-[2-(4-Benzo[d]isothiazol-3-yl-piperazin-1-yl)-ethyl]-6,6-dimethyl-1,2,5,6-tetrahydropyrrolo[3,2,1-ij]quinolin-4-one

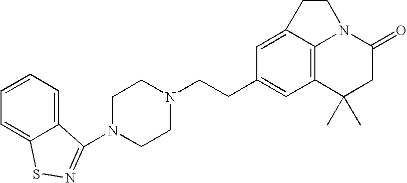

The product from Example 90C (1.0 g, 3.79 mmol) was reacted with 3-piperazin-1-yl-benzo[d]isothiazole hydrochloride (808 mg, 3.16 mmol) based on the procedure described for Example 88C. The title compound was taken up in methylene chloride and the solution treated with 4.0 N HCl solution in 1,4-dioxane to precipitate the hydrochloride salt. Yield=482 mg (32%); MS (APCI), (M+1)⁺=447. ¹H-NMR (DMSO-d₆, δ): 8.10 (dd, 2H, J=8.3, 8.1 Hz), 7.57 (t, 1H, J=7.6, 7.6 Hz), 7.45 (t, 1H, J=7.8, 7.3 Hz), 7.02 (s, 2H), 4.08 (d, 2H, J=13.2 Hz), 3.92 (t, 2H, J=8.3, 8.5 Hz), 3.66-3.02 (m, 12H), 2.47 (s, 2H), 1.20 (s, 6H). CHN: calculated for C₂₆H₃₀N₄OS 0.6 H₂O, C, 63.23%, H, 6.57%, N, 11.34%; found C, 62.94%, H, 6.61%, N, 10.91%.

### Example 91

### 6-{2-[4-(6-FLUORO-BENZO[D]ISOXAZOL-3-YL)-PIPERIDIN-1-YL]-ETHYL}-4S-METHYL-3,4-DIHYDRO-1H-QUINOLIN-2-ONE

The title compound was prepared from 6-fluoro-3-piperidin-4-yl-benzo[d]isoxazole hydrochloride (500 mg, 1.95 mmol. J. Med. Chem., 1985, 28, 761) and 6-(2-chloroethyl)-4S-methyl-3,4-dihydro-1H-quinolin-2-one (658 mg, 2.94 mmol, reference to example 2) based on the procedure described in Example 1C to give an amorphous solid. Yield=258 mg (32%); MS (APCI), (M+1)⁺=408, (M−1)⁺=406. ¹H-NMR (DMSO-d₆, δ): 9.98 (s, 1H), 7.97 (t, 1H, J=3.4, 5.1 Hz), 7.66 (d, 1H, J=9.3 Hz), 7.25 (t, 1H, J=9.0, 9.0 Hz), 7.04 (s, 1H), 6.97 (d, 1H, J=8.1 Hz), 6.73 (d, 1H, J=7.8 Hz), 3.12-2.96 (m, 4H), 2.67-2.47 (m, 5H), 2.15 (m, 3H), 1.99 (m, 2H), 1.82 (m, 2H), 1.14 (d, 3H, J=6.8 Hz). CHN: calculated for C₂₄H₂₆FN₃O₂, C, 70.74%, H, 6.43%, N, 10.31%; found, C, 70.22%, H, 6.54%, N, 9.99%. Optical rotation [α]^{D}₂₅=−3.6° (DMSO, c=10 mg/ml).

### Example 92

### 6-{2-[4-(6-FLUORO-BENZO[D]ISOXAZOL-3-YL)-PIPERIDIN-1-YL]-ETHYL}-4,4-DIMETHYL-3,4-DIHYDRO-1H-QUINOLIN-2-ONE

The title compound was prepared from 6-fluoro-3-piperidin-4-yl-benzo[d]isoxazole hydrochloride (500 mg, 1.95 mmol. J. Med. Chem., 1985, 28, 761) and 6-(2-chloroethyl)-4,4-dimethyl-3,4-dihydro-1H-quinolin-2-one (699 mg, 2.94 mmol, Example 5D) based on the procedure described in Example 1C to give a white, amorphous solid. Yield=319 mg (39%); MS (APCI), (M+1)⁺=422, (M−1)⁺=420. ¹H-NMR (DMSO-d₆, δ): 10.02 (s, 1H), 7.97 (q, 1H, J=5.1, 3.4, 5.4 Hz), 7.66 (d, 1H, J=9.0 Hz), 7.25 (t, 1H, J=9.3, 9.0 Hz), 7.13 (s, 1H), 6.97 (d, 1H, J=7.6 Hz), 6.74 (d, 1H, J=8.1 Hz), 3.15-3.00 (m, 3H), 2.68 (m, 4H), 2.29 (s, 2H), 2.17-1.76 (m, 6H), 1.18 (s, 6H). CHN: calculated for C₂₅H₂₈FN₃O₂, C, 71.24%, H, 6.70%, N, 9.97%; found C, 70.83%, H, 6.82%, N, 9.76%.

### Example 93

### 6-{2-[4-(6-FLUORO-BENZO[D]ISOTHIAZOL-3-YL)-PIPERIDIN-1-YL]-ETHYL}-4S-METHYL-3,4-DIHYDRO-1H-QUINOLIN-2-ONE

The title compound was prepared from 6-fluoro-3-piperidin-4-yl-benzo[d]isothiazole hydrochloride (500 mg, 1.83 mmol, WO 0160796 A1) and 6-(2-chloroethyl)-4S-methyl-3,4-dihydro-1H-quinolin-2-one (615 mg, 2.75 mmol, reference to Example 2) based on the procedure described in Example 1C to give a orange glass upon formation of the hydrochloride salt via methodology described earlier. Yield=339 mg (40%); MS (APCI), (M+1)⁺=424, (M−1)⁺=422. ¹H-NMR (DMSO-d₆, δ): 10.38 (br s, 1H), 10.08 (s, 1H), 8.31 (q, 1H, J=4.9, 3.9, 4.9 Hz), 8.08 (d, 1H, J=9.0 Hz), 7.42 (t, 1H, J=8.8, 9.0 Hz), 7.12 (s, 1H), 7.05 (d, 1H, J=7.8 Hz), 6.80 (d, 1H, J=8.1 Hz), 3.66-3.00 (m, 1OH), 2.55 (dd, 1H, J=5.9, 5.6 Hz), 2.18 (m, 5H), 1.16 (d, 3H, J=6.8 Hz). CHN: calculated for C₂₄H₂₆FN₃OS 1.1HCl, C, 62.17%, H, 5.89%, N, 9.06%; found, C, 61.79%, H, 6.00%, N, 8.93%. Optical rotation: [α]^{D}₂₅=−5.6° (DMSO, c=10 mg/ml).

### Example 94

### 6-{2-[4-(6-FLUORO-BENZO[D]ISOTHIAZOL-3-YL)-PIPERIDIN-1-YL]-ETHYL}-4,4-DIMETHYL-3,4-DIHYDRO-1H-QUINOLIN-2-ONE

The title compound was prepared from 6-fluoro-3-piperidin-4-yl-benzo[d]isothiazole hydrochloride (500 mg, 1.83 mmol, WO 0160796 A1) and_{—}6-(2-chloroethyl)-4,4-dimethyl-3,4-dihydro-1H-quinolin-2-one (654 mg, 2.75 mmol, Example 5D) based on the procedure described in Example 1C to give a white glass. Yield=339 mg (42%); MS (APCI), (M+1)⁺=438, (M−1)⁺=436. ¹H-NMR (DMSO-d₆, δ): 10.00 (s, 1H), 8.18 (q, 1H, J=4.9, 4.1, 4.9 Hz), 8.01 (d, 1H, J=6.8 Hz), 7.33 (t, 1H, J=6.6, 6.6 Hz), 7.11 (s, 1H), 6.95 (d, 1H, J=8.1 Hz), 6.72 (d, 1H, J=8.1 Hz), 3.26 (m, 1H), 2.99 (br d, 2H, J=11.2 Hz), 2.65 (t, 2H, J=7.1, 8.3 Hz), 2.49 (m, 2H), 2.26 (s, 2H), 2.13 (br t, 2H, J=10.5, 10.7 Hz), 1.86 (m, 4H), 1.16 (s, 6H). CHN: calculated for C₂₅H₂₈FN₃OS, C, 68.62%, H, 6.45%, N, 9.60%; found, C, 68.69%, H, 6.48%, N, 9.39%.

Examples 95 through 157 represent those title compounds prepared by combinatorial chemistry methodology.

### Example 95

### 2-{6-[2-(4-BENZO[D]ISOTHIAZOL-3-YL-PIPERAZIN-1-YL)-ETHYL]-3-METHYL-2-OXO-3,4-DIHYDRO-2H-QUINOLIN-1-YL}-ACETAMIDE

6-[2-(4-Benzo[d]isothiazol-3-yl-piperazin-1-yl)-ethyl]-3-methyl-3,4-dihydro-1H-quinolin-2-one was diluted to 0.50 M with anhydrous tetrahydrofuran, then delivered to an 8 mL vial via pipette (0.10 mmol). To the quinolinone solution was added potassium tert-butoxide (0.20 mmol, 1.0 M in tetrahydrofuran). The solution was stirred at ambient temperature for 0.5 h. 2-Bromo-acetamide was diluted to 0.50 M with tetrahydrofuran, and added to the quinolinone solution at room temperature (0.40 mmol). The solution was stirred overnight at 45 deg C., then cooled to room temperature. The following day an additional 0.20 mmol of 2-bromo-acetamide was added. The reaction was stirred overnight at 45 deg C. The reaction was partitioned with 2 mL ethyl acetate and 2 mL water. Organic phase was extracted and concentrated via HT-12 GeneVac. The crude was purified by HPLC (30×100 mm ODS-A C(18) 5 u column). 2-{6-[2-(4-Benzo[d]isothiazol-3-yl-piperazin-1-yl)-ethyl]-3-methyl-2-oxo-3,4-dihydro-2H-quinolin-1-yl}-acetamide was isolated in 100% purity @ 254 nm, LCMS (APCI) 464 [M+H]⁺.

Examples 96-157 were synthesized in combinatorial library format following the steps outlined in Example 95 on a 0.10 mmol scale using 6-[2-(4-Benzo[d]isothiazol-3-yl-piperazin-1-yl)-ethyl]-3-methyl-3,4-dihydro-1H-quinolin-2-one, 6-[2-(4-Benzo[d]isothiazol-3-yl-piperazin-1-yl)-ethyl]-3,3-dimethyl-3,4-dihydro-1H-quinolin-2-one, 6-(2-(4-Benzo[d]isothiazol-3-yl-piperazin-1-yl)-ethyl]-4-methyl-3,4-dihydro-1H-quinolin-2-one, 6-[2-(4-Benzo[d]isoxazol-3-yl-piperazin-1-yl)-ethyl]-4-methyl-3,4-dihydro-1H-quinolin-2-one, 6-[2-(4-Benzo[d]isothiazol-3-yl-piperazin-1-yl)-ethyl]-3,4-dimethyl-1H-quinolin-2-one, 6-[2-(4-Benzo[d]isoxazol-3-yl-piperazin-1-yl)-ethyl]-3,4-dimethyl-1H-quinolin-2-one, 6-[2-(4-Benzo[d]isoxazol-3-yl-piperazin-1-yl)-ethyl]-3,4-dimethyl-1H-quinolin-2-one, and 6-[2-(4-Benzo[d]isothiazol-3-yl-piperazin-1-yl)-ethyl]-4,4,8-trimethyl-3,4-dihydro-1H-quinolin-2-one with appropriate alkyl halide starting materials and potassium tert-butoxide. The crude products were purified by HPLC (30×100 mm ODS-A C(18) 5 u column).

The title compounds of Examples 96-157 were prepared by a procedure analogous to that described for Example 95.

### Example 96

### 2-{6-[2-(4-BENZO[D]ISOTHIAZOL-3-YL-PIPERAZIN-1-YL)-ETHYL]-3-METHYL-2-OXO-3,4-DIHYDRO-2H-QUINOLIN-1-YL}-PROPIONAMIDE

Isolated in 100% purity @ 254 nm; LCMS (APCI) 478 [M+H]⁺

### Example 97

### 2-{6-[2-(4-BENZO[D]ISOTHIAZOL-3-YL-PIPERAZIN-1-YL)-ETHYL]-3-METHYL-2-OXO-3,4-DIHYDRO-2H-QUINOLIN-1-YL}-N-PHENYL-PROPIONAMIDE

Isolated in 100% purity @ 254 nm; LCMS (APCI) 554 [M+H]⁺

### Example 98

### {6-[2-(4-BENZO[D]ISOTHIAZOL-3-YL-PIPERAZIN-1-YL)-ETHYL]-3-METHYL-2-OXO-3,4-DIHYDRO-2H-QUINOLIN-1-YL}-ACETIC ACID ETHYL ESTER

Isolated in 100% purity @ 254 nm; LCMS (APCI) 493 [M+H]⁺

### Example 99

### 6-[2-(4-BENZO[D]ISOTHIAZOL-3-YL-PIPERAZIN-1-YL)-ETHYL]-1-(3,3-DIMETHYL-2-OXO-BUTYL)-3-METHYL-3,4-DIHYDRO-1H-QUINOLIN-2-ONE

Isolated in 100% purity @ 254 nm; LCMS (APCI) 505 [M+H]⁺

### Example 100

### 6-[2-(4-BENZO[D]ISOTHIAZOL-3-YL-PIPERAZIN-1-YL)-ETHYL]-3-METHYL-1-(2-OXO-2-PHENYL-ETHYL)-3,4-DIHYDRO-1H-QUINOLIN-2-ONE

Isolated in 100% purity @ 254 nm; LCMS (APCI) 525 [M+H]⁺

### Example 101

### 6-[2-(4-BENZO[D]ISOTHIAZOL-3-YL-PIPERAZIN-1-YL)-ETHYL]-1-(2-METHOXY-ETHYL)-3-METHYL-3,4-DIHYDRO-1H-QUINOLIN-2-ONE

Isolated in 100% purity @ 254 nm; LCMS (APCI) 465 [M+H]⁺

### Example 102

### 2-{6-[2-(4-BENZO[D]ISOTHIAZOL-3-YL-PIPERAZIN-1-YL)-ETHYL]-3-METHYL-2-OXO-3,4-DIHYDRO-2H-QUINOLIN-1-YL}-PROPIONITRILE

Isolated in 100% purity @ 254 nm; LCMS (APCI) 460 [M+H]⁺

### Example 103

### 6-[2-(4-BENZO[D]ISOTHIAZOL-3-YL-PIPERAZIN-1-YL)-ETHYL]-1-ISOBUTYL-3-METHYL-3,4-DIHYDRO-1H-QUINOLIN-2-ONE

Isolated in 100% purity @ 254 nm; LCMS (APCI) 463 [M+H]⁺

### Example 104

### 2-{6-[2-(4-BENZO[D]ISOTHIAZOL-3-YL-PIPERAZIN-1-YL)-ETHYL]-3,3-DIMETHYL-2-OXO-3,4-DIHYDRO-2H-QUINOLIN-1-YL}-ACETAMIDE

Isolated in 100% purity @ 254 nm; LCMS (APCI) 478 [M+H]⁺

### Example 105

### {6-[2-(4-BENZO[D]ISOTHIAZOL-3-YL-PIPERAZIN-1-YL)-ETHYL]-3,3-DIMETHYL-2-OXO-3,4-DIHYDRO-2H-QUINOLIN-1-YL}-ACETIC ACID ETHYL ESTER

Isolated in 100% purity @ 254 nm; LCMS (APCI) 507 [M+H]⁺

### Example 106

### 6-[2-(4-BENZO[D]ISOTHIAZOL-3-YL-PIPERAZIN-1-YL)-ETHYL]-1-(3,3-DIMETHYL-2-OXO-BUTYL)-3,3-DIMETHYL-3,4-DIHYDRO-1H-QUINOLIN-2-ONE

Isolated in 100% purity @ 254 nm; LCMS (APCI) 519 [M+H]⁺

### Example 107

### 6-[2-(4-BENZO[D]ISOTHIAZOL-3-YL-PIPERAZIN-1-YL)-ETHYL]-3,3-DIMETHYL-1-(2-OXO-2-PHENYL-ETHYL)-3,4-DIHYDRO-1H-QUINOLIN-2-ONE

Isolated in 88% purity @ 254 nm; LCMS (APCI) 539 [M+H]⁺

### Example 108

### 6-[2-(4-BENZO[D]ISOTHIAZOL-3-YL-PIPERAZIN-1-YL)-ETHYL]-1-METHOXYMETHYL-3,3-DIMETHYL-3,4-DIHYDRO-1H-QUINOLIN-2-ONE

Isolated in 100% purity @ 254 nm; LCMS (APCI) 465 [M+H]⁺

### Example 109

### 6-[2-(4-BENZO[D]ISOTHIAZOL-3-YL-PIPERAZIN-1-YL)-ETHYL-1-(2-METHOXY-ETHYL)-3,3-DIMETHYL-3,4-DIHYDRO-1H-QUINOLIN-2-ONE

Isolated in 100% purity @ 254 nm; LCMS (APCI) 479 [M+H]⁺

### Example 110

### 2-{6-[2-(4-BENZO[D]ISOTHIAZOL-3-YL-PIPERAZIN-1-YL)-ETHYL]-3,3-DIMETHYL-2-OXO-3,4-DIHYDRO-2H-QUINOLIN-1-YL}-PROPIONITRILE

Isolated in 100% purity @ 254 nm; LCMS (APCI) 474 [M+H]⁺

### Example 111

### 6-[2-(4-BENZO[D]ISOTHIAZOL-3-YL-PIPERAZIN-1-YL)-ETHYL]-1-ETHYL-3,3-DIMETHYL-3,4-DIHYDRO-1H-QUINOLIN-2-ONE

Isolated in 100% purity @ 254 nm; LCMS (APCI) 449 [M+H]⁺

### Example 112

### 6-[2-(4-BENZO[D]ISOTHIAZOL-3-YL-PIPERAZIN-1-YL)-ETHYL]-1-ISOBUTYL-3,3-DIMETHYL-3,4-DIHYDRO-1H-QUINOLIN-2-ONE

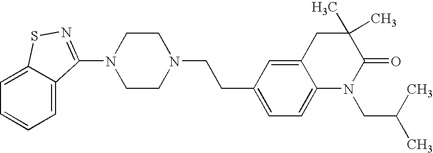

Isolated in 100% purity @ 254 nm, LCMS (APCI) 477 [M+H]⁺

### Example 113

### 2-{6-[2-(4-BENZO[D]ISOTHIAZOL-3-YL-PIPERAZIN-1-YL)-ETHYL]-4-METHYL-2-OXO-3,4-DIHYDRO-2H-QUINOLIN-1-YL}-PROPIONAMIDE

Isolated in 100% purity @ 254 nm; LCMS (APCI) 478 [M+H]⁺

### Example 114

### 2-{6-[2-(4-BENZO[D]ISOTHIAZOL-3-YL-PIPERAZIN-1-YL)-ETHYL]-4-METHYL-2-OXO-3,4-DIHYDRO-2H-QUINOLIN-1-YL}-N-PHENYL-PROPIONAMIDE

Isolated in 100% purity @ 254 nm; LCMS (APCI) 554 [M+H]⁺

### Example 115

### {6-[2-(4-BENZO[D]ISOTHIAZOL-3-YL-PIPERAZIN-1-YL)-ETHYL]-4-METHYL-2-OXO-3,4-DIHYDRO-2H-QUINOLIN-1-YL}-ACETIC ACID ETHYL ESTER

Isolated in 100% purity @ 254 nm; LCMS (APCI) 493 [M+H]⁺

### Example 116

### 6-[2-(4-BENZO[D]ISOTHIAZOL-3-YL-PIPERAZIN-1-YL)-ETHYL]-1-(3,3-DIMETHYL-2-OXO-BUTYL)-4-METHYL-3,4-DIHYDRO-1H-QUINOLIN-2-ONE

Isolated in 100% purity @ 254 nm; LCMS (APCI) 505 [M+H]⁺

### Example 117

### 6-[2-(4-BENZO[D]ISOTHIAZOL-3-YL-PIPERAZIN-1-YL)-ETHYL]-4-METHYL-1-(2-OXO-2-PHENYL-ETHYL)-3,4-DIHYDRO-1H-QUINOLIN-2-ONE

Isolated in 100% purity @ 254 nm; LCMS (APCI) 525 [M+H]⁺

### Example 118

### 2-{6-[2-(4-BENZO[D]ISOTHIAZOL-3-YL-PIPERAZIN-1-YL)-ETHYL]-4-METHYL-2-OXO-3,4-DIHYDRO-2H-QUINOLIN-1-YL}-PROPIONITRILE

Isolated in 100% purity @ 254 nm; LCMS (APCI) 460 [M+H]⁺

### Example 119

### 6-[2-(4-BENZO[D]ISOTHIAZOL-3-YL-PIPERAZIN-1-YL)-ETHYL]-1-ETHYL-4-METHYL-3,4-DIHYDRO-1H-QUINOLIN-2-ONE

Isolated in 100% purity @ 254 nm; LCMS (APCI) 435 [M+H]⁺

### Example 120

### 6-[2-(4-BENZO[D]ISOTHIAZOL-3-YL-PIPERAZIN-1-YL)-ETHYL]-4-METHYL-1-(2,2,2-TRIFLUORO-ETHYL)-3,4-DIHYDRO-1H-QUINOLIN-2-ONE

Isolated in 100% purity @ 254 nm; LCMS (APCI) 489 [M+H]⁺

### Example 121

### 6-[2-(4-BENZO[D]ISOTHIAZOL-3-YL-PIPERAZIN-1-YL)-ETHYL]-1-ISOBUTYL-4-METHYL-3,4-DIHYDRO-1H-QUINOLIN-2-ONE

Isolated in 100% purity @ 254 nm; LCMS (APCI) 463 [M+H]⁺

### Example 122

### 2-{6-[2-(4-BENZO[D]ISOXAZOL-3-YL-PIPERAZIN-1-YL)- ETHYL]-4-METHYL-2-OXO-3,4-DIHYDRO-2H-QUINOLIN-1-YL}-ACETAMIDE

Isolated in 89% purity @ 254 nm; LCMS (APCI) 448 [M+H]⁺

### Example 123

### 2-{6-[2-(4-BENZO[D]ISOXAZOL-3-YL-PIPERAZIN-1-YL)-ETHYL]-4-METHYL-2-OXO-3,4-DIHYDRO-2H-QUINOLIN-1-YL}-PROPIONAMIDE

Isolated in 97% purity @ 254 nm; LCMS (APCI) 462 [M+H]⁺

### Example 124

### 2-{6-[2-(4-BENZO[D]ISOXAZOL-3-YL-PIPERAZIN-1-YL)-ETHYL]-4-METHYL-2-OXO-3,4-DIHYDRO-2H-QUINOLIN-1-YL}-N-PHENYL-PROPIONAMIDE

Isolated in 100% purity @ 254 nm; LCMS (APCI) 538+H]⁺

### Example 125

### {6-[2-(4-BENZO[D]ISOXAZOL-3-YL-PIPERAZIN-1-YL)-ETHYL]-4-METHYL-2-OXO-3,4-DIHYDRO-2H-QUINOLI N-1-YL}-ACETIC ACID ETHYL ESTER

Isolated in 100% purity @ 254 nm; LCMS (APCI) 477 [M+H]⁺

### Example 126

### 6-[2-(4-BENZO[D]ISOXAZOL-3-YL-PIPERAZIN-1-YL)-ETHYL]-1-(3,3-DIMETHYL-2-OXO-BUTYL)-4-METHYL-3,4-DIHYDRO-1H-QUINOLIN-2-ONE

Isolated in 100% purity @ 254 nm; LCMS (APCI) 489 [M+H]⁺

### Example 127

### 6-[2-(4-BENZO[D]ISOXAZOL-3-YL-PIPERAZIN-1-YL)-ETHYL]-1-(2-METHOXY-ETHYL)-4-METHYL-3,4-DIHYDRO-1H-QUINOLIN-2-ONE

Isolated in 100% purity @ 254 nm; LCMS (APCI) 449 [M+H]⁺

### Example 128

### 2-{6-[2-(4-BENZO[D]ISOXAZOL-3-YL-PIPERAZIN-1-YL)-ETHYL]-4-METHYL-2-OXO-3,4-DIHYDRO-2H-QUINOLIN-1-YL}-PROPIONITRILE

Isolated in 100% purity @ 254 nm; LCMS (APCI) 444 [M+H]⁺

### Example 129

### 6-[2-(4-BENZO[D]ISOXAZOL-3-YL-PIPERAZIN-1-YL)- ETHYL]-1-ETHYL-4-METHYL-3,4-DIHYDRO-1H-QUINOLIN-2-ONE

Isolated in 100% purity @ 254 nm; LCMS (APCI) 419 [M+H]⁺

### Example 130

### 6-[2-(4-BENZO[D]ISOXAZOL-3-YL-PIPERAZIN-1-YL)-ETHYL]-1-ISOBUTYL-4-METHYL-3,4-DIHYDRO-1H-QUINOLIN-2-ONE

Isolated in 100% purity @ 254 nm; LCMS (APCI) 447 [M+H]⁺

### Example 131

### 6-[2-(4-BENZO[D]ISOTHIAZOL-3-YL-PIPERAZIN-1-YL)-ETHYL]-1-(3,3-DIMETHYL-2-OXO-BUTYL)-3,4-DIMETHYL-1H-QUINOLIN-2-ONE

Isolated in 100% purity @ 254 nm; LCMS (APCI) 517 [M+H]⁺

### Example 132

### 6-[2-(4-BENZO[D]ISOTHIAZOL-3-YL-PIPERAZIN-1-YL)-ETHYL]-1-[2-(2-HYDROXY-ETHOXY)-ETHYL]-3,4-DIMETHYL-1H-QUINOLIN-2-ONE

Isolated in 100% purity @ 254 nm; LCMS (APCI) 507 [M+H]⁺

### Example 133

### 6-[2-(4-BENZO[D]ISOTHIAZOL-3-YL-PIPERAZIN-1-YL)-ETHYL]-1-(2-METHOXY-ETHYL)-3,4-DIMETHYL-1H-QUINOLIN-2-ONE

Isolated in 100% purity @ 254 nm; LCMS (APCI) 477 [M+H]⁺

### Example 134

### 6-[2-(4-BENZO[D]ISOTHIAZOL-3-YL-PIPERAZIN-1-YL)-ETHYL]-1-ETHYL-3,4-DIMETHYL-1H-QUINOLIN-2-ONE

Isolated in 100% purity @ 254 nm; LCMS (APCI) 447 [M+H]⁺

### Example 135

### 6-[2-(4-BENZO[D]ISOTHIAZOL-3-YL-PIPERAZIN-1-YL)-ETHYL]-3,4-DIMETHYL-1-(2,2,2-TRIFLUORO-ETHYL)-1H-QUINOLIN-2-ONE

Isolated in 100% purity @ 254 nm; LCMS (APCI) 501 [M+H]⁺

### Example 136

### 6-[2-(4-BENZO[D]ISOTHIAZOL-3-YL-PIPERAZIN-1-YL)-ETHYL]-1-ISOBUTYL-3,4-DIMETHYL-1H-QUINOLIN-2-ONE

Isolated in 100% purity @ 254 nm; LCMS (APCI) 475 [M+H]⁺

### Example 137

### 2-{6-[2-(4-BENZO[D]ISOXAZOL-3-YL-PIPERAZIN-1-YL)-ETHYL]-3,4-DIMETHYL-2-OXO-2H-QUINOLIN-1-YL}-ACETAMIDE

Isolated in 94% purity @ 254 nm; LCMS (APCI) 460 [M+H]⁺

### Example 138

### 2-{6-[2-(4-BENZO[D]ISOXAZOL-3-YL-PIPERAZIN-1-YL)-ETHYL]-3,4-DIMETHYL-2-OXO-2H-QUINOLIN-1-YL}-PROPIONAMIDE

Isolated in 100% purity @ 254 nm; LCMS (APCI) 474 [M+H]⁺

### Example 139

### {6-[2-(4-BENZO[D]ISOXAZOL-3-YL-PIPERAZIN-1-YL)-ETHYL]-3,4-DIMETHYL-2-OXO-2H-QUINOLIN-1-YL}-ACETIC ACID ETHYL ESTER

Isolated in 100% purity @ 254 nm; LCMS (APCI) 489 [M+H]⁺

### Example 140

### 2-{6-[2-(4-BENZO[D]ISOXAZOL-3-YL-PIPERAZIN-1-YL)-ETHYL]-3,4-DIMETHYL-2-OXO-2H-QUINOLIN-1-YL}-PROPIONIC ACID METHYL ESTER

Isolated in 100% purity @ 254 nm; LCMS (APCI) 489 [M+H]⁺

### Example 141

### 6-[2-(4-BENZO[D]ISOXAZOL-3-YL-PIPERAZIN-1-YL)-ETHYL]-1-(3,3-DIMETHYL-2-OXO-BUTYL)-3,4-DIMETHYL-1H-QUINOLIN-2-ONE

Isolated in 100% purity @ 254 nm; LCMS (APCI) 501 [M+H]⁺

### Example 142

### 6-[2-(4-BENZO[D]ISOXAZOL-3-YL-PIPERAZIN-1-YL)-ETHYL]-3,4-DIMETHYL-1-(2-OXO-2-PHENYL-ETHYL)-1H-QUINOLIN-2-ONE

Isolated in 100% purity @ 254 nm; LCMS (APCI) 521 [M+H]⁺

### Example 143

### 6-[2-(4-BENZO[D]ISOXAZOL-3-YL-PIPERAZIN-1-YL)-ETHYL]-1-(2-METHOXY-ETHYL)-3,4-DIMETHYL-1H-QUINOLIN-2-ONE

Isolated in 100% purity @ 254 nm; LCMS (APCI) 461 [M+H]⁺

### Example 144

### 2-{6-[2-(4-BENZO[D]ISOXAZOL-3-YL-PIPERAZIN-1-YL)-ETHYL]-3,4-DIMETHYL-2-OXO-2H-QUINOLIN-1-YL}-PROPIONITRILE

Isolated in 96% purity @ 254 nm; LCMS (APCI) 456 [M+H]⁺

### Example 145

### 6-[2-(4-BENZO[D]ISOXAZOL-3-YL-PIPERAZIN-1-YL)-ETHYL]-1-ETHYL-3,4-DIMETHYL-1H-QUINOLIN-2-ONE

Isolated in 100% purity @ 254 nm; LCMS (APCI) 431 [M+H]⁺

### Example 146

### 6-[2-(4-BENZO[D]ISOXAZOL-3-YL-PIPERAZIN-1-YL)-ETHYL]-3,4-DIMETHYL-1-(2,2,2-TRIFLUORO-ETHYL)-1H-QUINOLIN-2-ONE

Isolated in 100% purity @ 254 nm; LCMS (APCI) 485 [M+H]⁺

### Example 147

### {6-[2-(4-BENZO[D]ISOTHIAZOL-3-YL-PIPERAZIN-1-YL)-ETHYL]-4,4,8-TRIMETHYL-2-OXO-3,4-DIHYDRO-2H-QUINOLIN-1-YL}-ACETIC ACID ETHYL ESTER

Isolated in 96% purity @ 254 nm; LCMS (APCI) 521 [M+H]⁺

### Example 148

### 6-[2-(4-BENZO[D]ISOTHIAZOL-3-YL-PIPERAZIN-1-YL)-ETHYL]-4,4,8-TRIMETHYL-1-PENTYL-3,4-DIHYDRO-1H-QUINOLIN-2-ONE

Isolated in 100% purity @ 254 nm; LCMS (APCI) 505 [M+H]⁺

### Example 149

### 6-[2-(4-BENZO[D]ISOTHIAZOL-3-YL-PIPERAZIN-1-YL)-ETHYL]-4,4,8-TRIMETHYL-1-(3-METHYL-BUTYL)-3,4-DIHYDRO-1H-QUINOLIN-2-ONE

Isolated in 100% purity @ 254 nm; LCMS (APCI) 505 [M+H]⁺

### Example 150

### 6-[2-(4-BENZO[D]ISOTHIAZOL-3-YL-PIPERAZIN-1-YL)-ETHYL]-1-(2-ETHYL-BUTYL)-4,4,8-TRIMETHYL-3,4-DIHYDRO-1H-QUINOLIN-2-ONE

Isolated in 100% purity @ 254 nm; LCMS (APCI) 519 [M+H]⁺

### Example 151

### 6-[2-(4-BENZO[D]ISOTHIAZOL-3-YL-PIPERAZIN-1-YL)-ETHYL]-1-(2-ETHOXY-ETHYL)-4,4,8-TRIMETHYL-3,4-DIHYDRO-1H-QUINOLIN-2-ONE

Isolated in 96% purity @ 254 nm; LCMS (APCI) 507 [M+H]⁺

### Example 152

### 6-[2-(4-BENZO[D]ISOTHIAZOL-3-YL-PIPERAZIN-1-YL)-ETHYL]-1-(2,2-DIMETHYL-PROPYL)-4,4,8-TRIMETHYL-3,4-DIHYDRO-1H-QUINOLIN-2-ONE

Isolated in 93% purity @ 254 nm; LCMS (APCI) 505 [M+H]⁺

### Example 153

### 6-[2-(4-BENZO[D]ISOTHIAZOL-3-YL-PIPERAZIN-1-YL)-ETHYL]-1-CYCLOHEXYLMETHYL-4,4,8-TRIMETHYL-3,4-DIHYDRO-1H-QUINOLIN-2-ONE

Isolated in 100% purity @ 254 nm; LCMS (APCI) 531 [M+H]⁺

### Example 154

### 6-[2-(4-BENZO[D]ISOTHIAZOL-3-YL-PIPERAZIN-1-YL)- ETHYL]-1-CYCLOBUTYLMETHYL-4,4,8-TRIMETHYL-3,4-DIHYDRO-1H-QUINOLIN-2-ONE

Isolated in 100% purity @ 254 nm; LCMS (APCI) 503 [M+H]⁺

### Example 155

### 6-[2-(4-BENZO[D]ISOTHIAZOL-3-YL-PIPERAZIN-1-YL)-ETHYL]-1-ISOBUTYL-4,4,8-TRIMETHYL-3,4-DIHYDRO-1H-QUINOLIN-2-ONE

Isolated in 100% purity @ 254 nm; LCMS (APCI) 491 [M+H]⁺

### Example 156

### 6-[2-(4-BENZO[D]ISOTHIAZOL-3-YL-PIPERAZIN-1-YL)-ETHYL]-1-BUTYL-4,4,8-TRIMETHYL-3,4-DIHYDRO-1H-QUINOLIN-2-ONE

Isolated in 100% purity @ 254 nm; LCMS (APCI) 491 [M+H]⁺

### Example 157

### 6-[2-(4-BENZO[D]ISOTHIAZOL-3-YL-PIPERAZIN-1-YL)-ETHYL]-1-CYCLOBUTYL-4,4,8-TRIMETHYL-3,4-DIHYDRO-1H-QUINOLIN-2-ONE

Isolated in 95% purity @ 254 nm; LCMS (APCI) 489 [M+H]⁺

### Example 158

### 6-{2-[4-(5-FLUORO-BENZO[D]ISOXAZOL-3-YL)-PIPERAZIN-1-YL]-ETHYL}-3,4-DIMETHYL-1H-QUINOLIN-2-ONE (3)

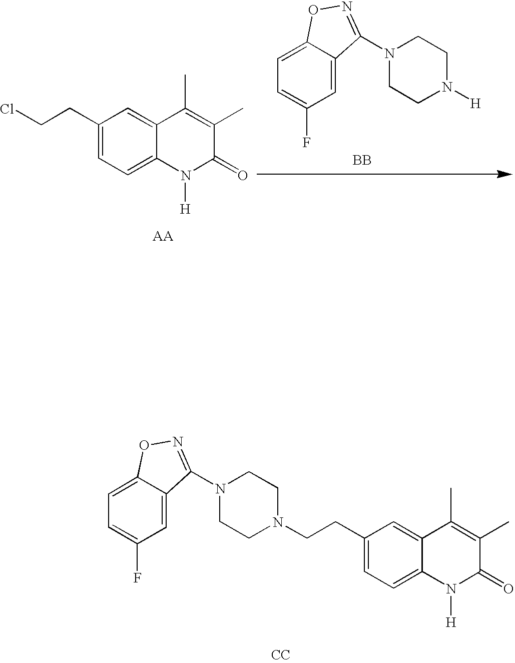

Referring to the scheme immediately above, in a 10 mL microwave reaction vial was combined 0.25 g (0.001 mol) AA, 0.35 g K₂CO₃ (2.5 eq, 0.0025 mol), 0.18 g potassium iodide (KI) (1 eq, 0.001 mol), 4 mL CH₃CN, and 0.35 g BB, 5-Fluoro-3-piperazin-1-yl-benzo[d]isoxazole, (1.5 eq, 0.0015 mol). The reaction vessel was heated to 150° C. for 5400 seconds in a microwave reactor, cooled to rt, poured into water and filtered. The resulting solid was dried in vacuo at 60° C. for 24 h. This gave 0.31 g of crude product (73% crude yield). Approximately 82 mg of this material was purified by preparative HPLC (YMC 30×100 mm ODS-A 5 uM C18, eluting with CH₃CN/H₂O+0.05% TFA) to give 42 mg of a solid that was gauged to be 100% pure by HPLC @ 254 and 214 nM. ¹H NMR (400 MHz, DMSO-d₆) δ ppm 2.1 (s, 3H), 2.4 (s, 3H), 3.1 (d, J=8.1 Hz, 2H), 3.3 (d, J=7.8 Hz, 6H), 3.7 (d, J=6.1 Hz, 2H), 4.1 (d, J=4.6 Hz, 2H), 7.2 (s, 1H), 7.3 (s, 1H), 7.5 (m, 1H), 7.6 (s, 1H), 7.7 (m, 1H), 8.0 (d, J=2.4 Hz, 1H), 11.7 (s, 1H). MS [M+H]⁺=421.

### Example 159

### 6-{2-[4-(6-FLUORO-BENZO[D]ISOXAZOL-3-YL)-PIPERAZIN-1-YL]-ETHYL}-3,4-DIMETHYL-1H-QUINOLIN-2-ONE

6-{2-[4-(6-Fluoro-benzo[d]isoxazol-3-yl)-piperazin-1-yl]-ethyl}-3,4-dimethyl-1H-quinolin-2-one was prepared as in Example 158 employing 6-Fluoro-3-piperazin-1-yl-benzo[d]isoxazole hydrochloride; HPLC: 100% purity @ 254 and 214 nM, MS [M+H]⁺=421.

### Example 160

### 6-{2-[4-(5-CHLORO-BENZO[D]ISOXAZOL-3-YL)-PIPERAZIN-1-YL]-ETHYL}-3,4-DIMETHYL-1H-QUINOLIN-2-ONE

6-{2-[4-(5-Chloro-benzo[d]isoxazol-3-yl)-piperazin-1-yl]-ethyl}-3,4-dimethyl-1H-quinolin-2-one was prepared as in Example 158 employing 5-Chloro-3-piperazin-1-yl-benzo[d]isoxazole. HPLC: 100% purity @ 254 and 214 nM, MS [M+H]⁺=437.

### Example 161

### 6-{2-[4-(5-METHOXY-BENZO[D]ISOTHIAZOL-3-YL)-PIPERAZIN-1-YL]-ETHYL}-3,4-DIMETHYL-1H-QUINOLIN-2-ONE

6-{2-[4-(5-Methoxy-benzo[d]isothiazol-3-yl)-piperazin-1-yl]-ethyl}-3,4-dimethyl-1H-quinolin-2-one was prepared as in Example 158 employing 5-Methoxy-3-piperazin-1-yl-benzo[d]isothiazole; HPLC: 100% purity @ 254 and 214 nM, MS [M+H]⁺=449.

### Example 162

### 6-{2-[4-(7-FLUORO-BENZO[D]ISOTHIAZOL-3-YL)-PIPERAZIN-1-YL]-ETHYL}-3,4-DIMETHYL-1H-QUINOLIN-2-ONE

6-{2-[4-(7-Fluoro-benzo[d]isothiazol-3-yl)-piperazin-1-yl]-ethyl}-3,4-dimethyl-1H-quinolin-2-one was prepared as in Example 158 employing 7-Fluoro-3-piperazin-1-yl-benzo[d]isothiazole hydrochloride. HPLC: 100% purity @ 254 and 214 nM, MS [M+H]⁺=437.

### Example 163

### 6-{2-[4-(6-FLUORO-BENZO[D]ISOTHIAZOL-3-YL)-PIPERAZIN-1-YL]-ETHYL}-3,4-DIMETHYL-1H-QUINOLIN-2-ONE

6-{2-[4-(6-Fluoro-benzo[d]isothiazol-3-yl)-piperazin-1-yl]-ethyl}-3,4-dimethyl-1H-quinolin-2-one was prepared as in Example 158 employing 6-Fluoro-3-piperazin-1-yl-benzo[d]isothiazole. HPLC: 100% purity @ 254 and 214 nM, MS [M+H]⁺=437.

### Example 164

### 6-{2-[4-(5-FLUORO-BENZO[D]ISOTHIAZOL-3-YL)-PIPERAZIN-1-YL]-ETHYL}-3,4-DIMETHYL-1H-QUINOLIN-2-ONE

6-{2-[4-(5-Fluoro-benzo[d]isothiazol-3-yl)-piperazin-1-yl]-ethyl}-3,4-dimethyl-1H-quinolin-2-one was prepared as in Example 158 employing 5-Fluoro-3-piperazin-1-yl-benzo[d]isothiazole. HPLC: 100% purity @ 254 and 214 nM, MS [M+H]⁺=437.

### Example 165

### 6-{2-[4-(6-FLUORO-BENZO[D]ISOTHIAZOL-3-YL)-PIPERIDIN-1-YL]-ETHYL}-3,4-DIMETHYL-1H-QUINOLIN-2-ONE

6-{2-[4-(6-Fluoro-benzo[d]isothiazol-3-yl)-piperidin-1-yl]-ethyl}-3,4-dimethyl-1H-quinolin-2-one was prepared as in Example 158 employing 6-Fluoro-3-piperidin-4-yl-benzo[d]isothiazole. HPLC: 100% purity @ 254 and 214 nM, MS [M+H]⁺=436.

### Example 166

### 6-{2-[4-(6-FLUORO-BENZO[D]ISOXAZOL-3-YL)-PIPERIDIN-1-YL]-ETHYL}-3,4-DIMETHYL-1H-QUINOLIN-2-ONE

6-{2-[4-(6-Fluoro-benzo[d]isoxazol-3-yl)-piperidin-1-yl]-ethyl}-3,4-dimethyl-1H-quinolin-2-one was prepared as in Example 158 employing 6-Fluoro-3-piperidin-4-yl-benzo[d]isoxazole. HPLC: 100% purity @ 254 and 214 nM, MS [M+H]⁺=420.

### Example 167

### 6-{2-[4-(1H-INDAZOL-3-YL)-PIPERAZIN-1-YL]-ETHYL}-3,4-DIMETHYL-1H-QUINOLIN-2-ONE

3-piperazin-1-yl-1H-indazole hydrochloride (382 mg, 1.60 mmol) was reacted with the compound prepared in step B of Example 9 (259 mg, 1.1 mmol) based on the procedure given in step C of Example 1 to give the title compound which precipitated out of solution as an amorphous solid. The product obtained was purified by elution through a flash column (silica gel 60, 230-400 mesh, 100:8:1, CH₂Cl:EtOH:NH₄OH) to give an off-white, foamy solid, yield=113 mg (26%). MP: 265.5-268.1° C. ¹H-NMR (DMSO-d₆, δ): 2.08 (s, 3H), 2.38 (s, 3H), 2.61 (m, 5H), 2.82 (m, 2H), 3.30 (m, 4H), 6.93 (t, J=7.45 Hz, 1H), 7.17 (d, J=8.30 Hz, 1H), 7.24 (m, 1H), 7.32 (m, 2H), 7.60 (s,1H), 7.71 (d, J=7.82 Hz, 1H).

### Example 168

### 8-[2-(4-BENZO[D]ISOTHIAZOL-3-YL-PIPERAZIN-1-YL)-ETHYL]-6-METHYL-3,5-DIHYDRO-1H-FURO[3,4-C]QUINOLIN-4-ONE

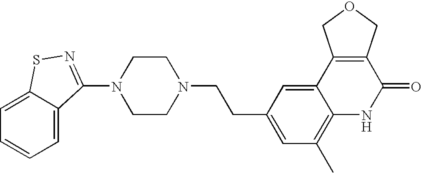

A. 1,4,7-Trioxa-spiro[4.4]nonane-9-carboxylic acid

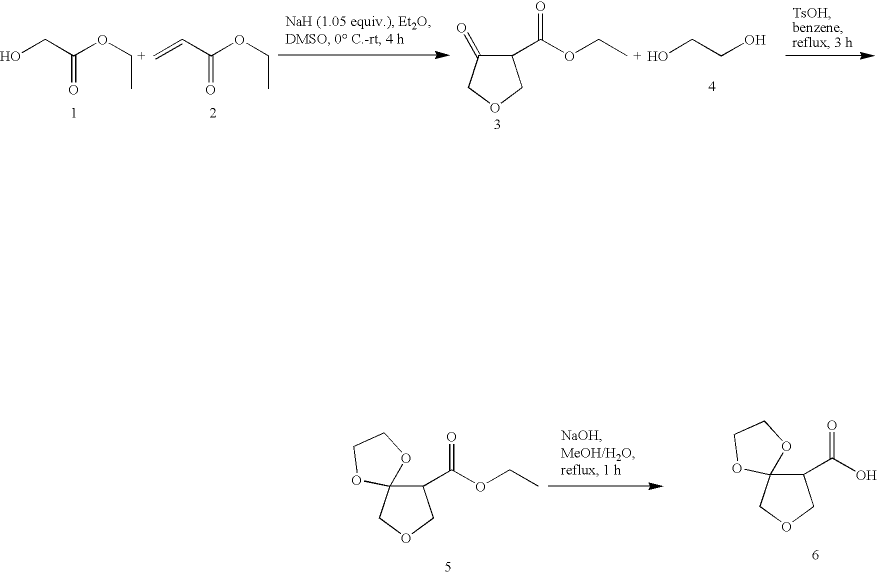

Referring to the scheme immediately above, to a suspension of sodium hydride (60% in oil) (42.0 g, 1.05 mol) in diethyl ether (800 mL) was added ethyl glycolate (100 g, 0.96 mol) over a period of 1 hour at room temperature. The suspension was then stirred for an additional 0.5 hour and evaporated under vacuum. To the resulting solid, dimethyl sulfoxide (200 mL) was added. It was then cooled to 0° C. and a solution of ethyl acrylate (115.10 g, 1.15 mol) in dimethyl sulfoxide (100 mL) was added in portions with vigorous stirring. The suspension was allowed to warm to room temperature and stirred for 3 hours. The reaction mixture was cautiously poured into ice cold aqueous solution of sulfuric acid (5%, 300 mL), extracted with ether (3×100 mL), dried over MgSO₄, evaporated and chromatographed on silica gel (hexane/ethyl acetate, 10:1) to afford compound 3 (70.0 g, 46%). ¹H NMR (400 MHz, CDCl₃) δ 4.55-4.40 (m, 2H), 4.30-4.20 (m, 2H), 4.10-3.95 (m, 2H), 3.50 (t, 1H), 1.15 (t, 3H).

Compound 3 (18.4 g, 0.11 mol), ethylene glycol (15.0 g, .22 mol), p-toluenesulfonic acid (2.2 g, 0.01 mol) and benzene (30 mL) were taken into a flask fitted with Dean-Stark and a condenser. The mixture was heated to reflux for 2 hours, allowed to cool to room temperature, and solvent was removed under vacuum. The viscous residue was diluted with ether (100 mL). The solution was washed with water, aqueous sodium bicarbonate, dried over MgSO₄. It was then evaporated to obtain compound 5 (14.9 g, 63%). ¹H NMR (400 MHz, CDCl₃) δ 4.30-4.10 (m, 6H), 4.00-3.85 (m, 4H), 3.10 (t, 1H), 1.15 (t, 3H).

A mixture of 5 (15.0 g, 74.2 mmol), NaOH (111.0 mmol, 4.5 g in 10 mL of water) and methanol (50 mL) was heated to reflux for 1 hour. The mixture was cooled to room temperature, methanol was removed under vacuum. The residue was acidified with dilute hydrochloric acid and extracted with ethyl acetate (3×50 mL). The combined extracts were dried over MgSO₄ and evaporated to afford compound 6 (9.1 g, 70%). ¹H NMR (400 MHz, DMSO-d₆) δ 12.4 (br s,1H), 4.05-3.95 (m, 2H), 3.90-3.85 (m, 4H), 3.65 (d, 1H), 3.55 (d, 1H), 3.10 (t, 1H).

B. 8-[2-(4-Benzo[d]isothiazol-3-yl-piperazin-1-yl)-ethyl]-6-methyl-3,5-dihydro-1H-furo[3,4-c]quinolin-4-one

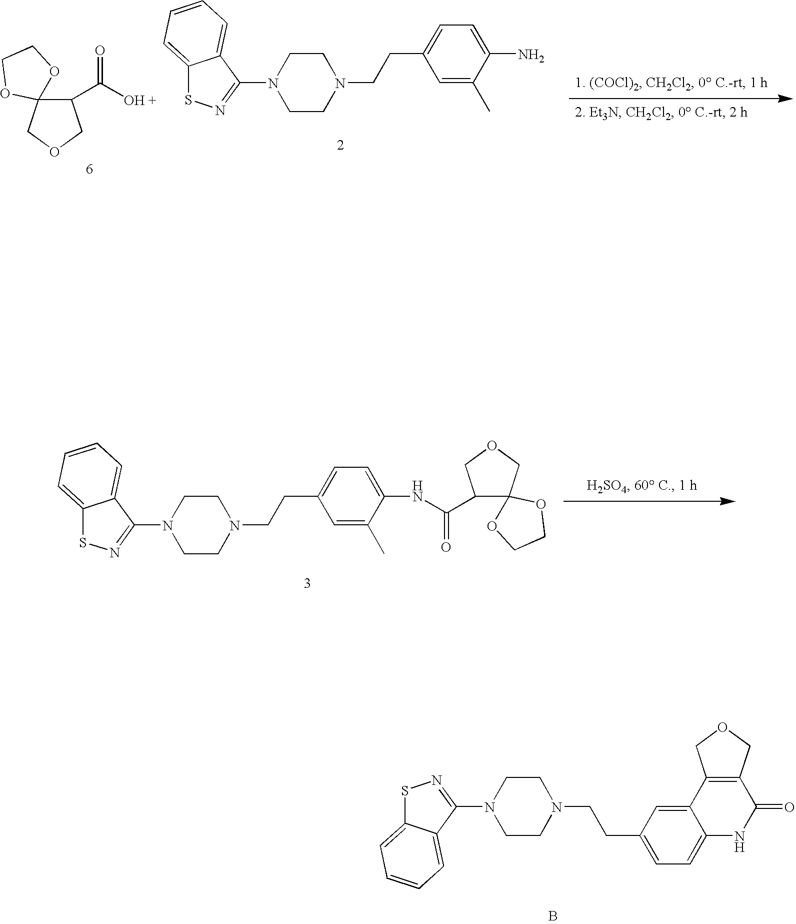

To a solution of 6 (1.18 g, 6.8 mmol) in a mixture of dimethyl formamide (0.1 mL) and dichloromethane (20 mL) at 0° C. was added oxalyl chloride (1.16 g, 11.9 mmol) slowly. It was then allowed to warm to room temperature and stirred for 1 hour. The mixture was evaporated under vacuum and was dissolved in dichloromethane (10 mL). This solution was then added to a mixture of 2 (2.0 g, 3.6 mmol) and triethyl amine (2.5 g, 24.6 mol) in dichloromethane (20 mL) at 0° C. The mixture was allowed to warm to room temperature and stirred for 2 hours and quenched with water. The organic layer was separated out, washed with brine, dried over MgSO₄, concentrated, and chromatographed over silica gel (methanol/ethyl acetate, 1:10) to obtain compound 3 (0.71 g, 24%). ¹H NMR (400 MHz, CDCl₃): δ 7.95-7.75 (m, 2H), 7.50-7.35 (m, 2H), 7.45 (t, 1H), 7.35 (t, 1H), 7.10-7.05 (m, 2H), 4.40 (dd, 1H), 4.15 (dd, 1H), 4.10-4.00 (m, 4H), 3.95 (d, 1H), 3.80 (d, 1H), 3.65-3.55 (m, 4H), 3.15 (dd, 1H), 2.85-2.60 (m, 8H), 2.25 (s, 3H).

A mixture of compound 3 (0.70 g, 1.40 mmol) and concentrated sulfuric acid (5 mL) was heated at 60° C. for 45 min. The mixture was cooled to room temperature and poured into ice to obtain a gum after filtration. The gum was suspended in methanol using a sonic bath, methanol was removed under vacuum. To the residue was added triethyl amine (10 mL) and refluxed for 15 min. The reaction mixture was evaporated under vacuum and chromatographed over silica gel (methanol/ethyl acetate, 10:100) to obtain compound B (0.518 g, 82%). ¹H NMR (400 MHz, DMSO-d₆): δ 10.95 (s, 1H), 8.05 (d, 2H), 7.60-7.55 (m, 1H), 7.50-7.40 (m, 1H), 7.30 (s, 1H), 7.20 (s, 1H), 5.35-5.25 (m, 2H), 5.00-4.95 (m, 2H), 3.50-3.30 (m, 4H), 2.90-2.55 (m, 8H), 2.45 (s, 3H).

### Example 169

### 8-[2-(4-BENZO[D]ISOTHIAZOL-3-YL-PIPERAZIN-1-YL)-ETHYL]-3,5-DIHYDRO-1H-FURO[3,4-C]QUINOLIN-4-ONE

The title compound was prepared in a fashion analogous to that described above for Example 168 from 4-[2-(4-Benzo[d]isothiazol-3-yl-piperazin-1-yl)-ethyl]-phenylamine. ¹H NMR (400 MHz, DMSO-d₆) δ 11.80 (br s, 1H), 8.20-8.00 (m, 2H), 7.60-7.20 (m, 5H), 5.25 (br s, 2H), 4.95 (br s, 2H), 3.60-3.40 (m, 4H), 2.90-2.50 (m, 8H).

### Example 170

### 8-[3-(4-BENZO[D]ISOTHIAZOL-3-YL-PIPERAZIN-1-YL)-PROPYL]-3,5-DIHYDRO-1H-FURO[3,4-C]QUINOLIN-4-ONE

The title compound was prepared in a fashion analogous to that described above for Example 168 from 4-[3-(4-Benzo[d]isothiazol-3-yl-piperazin-1-yl)-propyl]-phenylamine. ¹H NMR (400 MHz, CDCl₃) δ 11.85 (s, 1H), 8.15 (t, 2H), 7.60 (t, 1H), 7.55-7.35 (m, 4H), 5.20 (br s, 2H), 5.00 (br s, 2H), 3.50 (br s, 4H), 2.75 (t, 2H), 2.60 (br s, 4H), 2.40 (t, 2H), 1.95-1.80 (m, 2H).

### Example 171

### 8-[2-(4-BENZO[D]ISOXAZOL-3-YL-PIPERAZIN-1-YL)-ETHYL]-3,5-DIHYDRO-1H-FURO[3,4-C]QUINOLIN-4-ONE

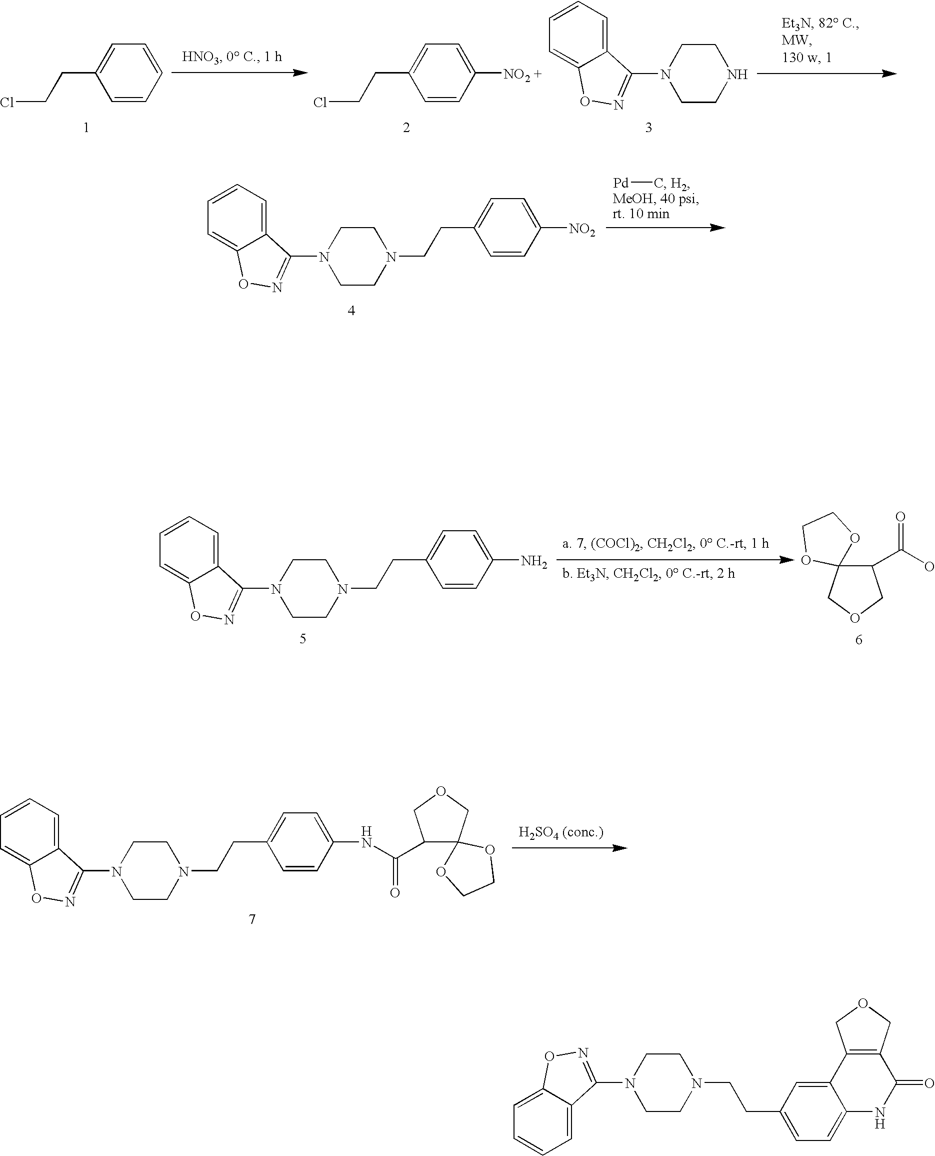

To 1-chloro-2-phenylethane (20 g, 0.14 mol) at 0° C. was added fuming nitric acid (20 ml) dropwise. The mixture was stirred at the same temperature for an additional 45 min. The reaction was quenched cautiously with water (200 mL). It was extracted with dichloromethane (100 mL), dried over MgSO₄, and crystallized from chloroform/hexane to obtain 2 (8.0 g, 30%). ¹H NMR (400 MHz, CDCl₃) δ 8.20 (d, 2H), 7.40 (d, 2H), 3.75 (t, 2H), 3.20 (t, 2H).

A mixture of 2 (2.3 g, 12.4 mmol), 3 (2.0 g, 8.4 mmol), triethylamine (5.0 g, 49.6 mmol), sodium iodide (7.4 g, 49.6 mmol), and acetonitrile (45 mL) was heated at 82° C. for 1 h in a microwave oven. It was then allowed to cool to room temperature and solvents were removed under vacuum. The residue was diluted with ethyl acetate (200 mL), washed with water, dried over MgSO₄, and evaporated. The crude material was purified by column chromatography (ethyl acetate/hexane, 50:50) to obtain 4 (1.8 g, 61%). ¹H NMR (400 MHz, CDCl₃) δ 8.20 (d, 2H), 7.70 (d, 1H), 7.50-7.45 (m, 2H), 7.35 (d, 2H), 7.25 (t, 1H), 3.65-3.55 (m, 4H), 3.00-2.90 (m, 2H), 2.80-2.65 (m, 6H).

A mixture of 4 (0.9 g, 3.0 mmol, in 10 mL of tetrahydrofuran) and palladium on carbon (1.6 g, 10%) in methanol (50.0 mL) was hydrogenated at 40 psi, at room temperature for 10 min. The reaction mixture was filtered through celite. The filtrate was evaporated and chromatographed on silica gel (dichloromethane) to obtain 5 (0.81 g, 50%). ¹H NMR (400 MHz, CDCl₃) 6 7.60 (d, 1H), 7.45-7.35 (m, 2H), 7.20-7.05 (m, 1H), 6.95 (d, 2H), 6.55 (d, 2H), 3.60-3.45 (m, 6H), 2.75-2.55 (m, 6H), 2.55-2.45 (m, 2H).

An analogous procedure, as described for compound 1,4,7-Trioxa-spiro[4.4]nonane-9-carboxylic acid {4-[2-(4-benzo[d]isothiazol-3-yl-piperazin-1-yl)-ethyl]-2-methyl-phenyl}-amide, was used to prepare compound 7 (1.0 g, 83%) from 5 (0.65 g, 3.75 mmol) and 7 (0.80 g, 2.50 mmol). ¹H NMR (400 MHz, CDCl₃) δ 7.85 (s, 1H), 7.70 (d, 1H), 7.55-7.05 (m, 3H), 7.40-7.15 (m, 4H), 4.40-3.60 (m, 14H), 3.15 (t, 1H), 2.80-2.45 (m, 6H).

An analogous procedure, as described for compound 8-[2-(4-Benzo[d]isothiazol-3-yl-piperazin-1-yl)-ethyl]-6-methyl-3,5-dihydro-1H-furo[3,4-c]quinolin-4-one, was used to prepare compound the desired product (0.25 g, 57%) from 7 (1.0 g, 2.10 mmol). ¹H NMR (400 MHz, CDCl₃) δ 11.80 (s, 1H), 8.00 (d, 1H), 7.60 (d, 2H), 7.45 (d, 1H), 7.40-7.25 (m, 3H), 5.30 (br s, 2H), 4.95 (br s, 2H), 3.50 (br s, 4H), 2.85 (t, 2H), 2.75-2.55 (br s, 6H).

### Example 172

### 8-[3-(4-BENZO[D]ISOTHIAZOL-3-YL-PIPERAZIN-1-YL)-PROPYL]-2-METHYL-1,2,3,5-TETRAHYDRO-PYRROLO[3,4-C]QUINOLIN-4-ONE

A. N-{4-[3-(4-Benzo[d]isothiazol-3-yl-piperazin-1-yl)-propyl]-phenyl}-acrylamide

To a solution of 4-[3-(4-Benzo[d]isothiazol-3-yl-piperazin-1-yl)-propyl]-phenylamine (2.43 g, 6.9 mmol) in 40 mL of dichloromethane containing triethylamine (0.84 g, 8.3 mmol) was added acryloyl chloride (0.69 g, 7.6 mmol) dropwise at 0° C. with stirring under nitrogen. The reaction mixture was allowed to warm to room temperature, and stirring continued for 2 hours, then 100 mL of dichloromethane was added. The mixture was washed with 10 mL of saturated sodium bicarbonate, brine (2×20 mL), dried over sodium sulphate, and concentrated to yield a yellowish sticky mass (2.80 g, quantitative yield) which was pure enough for the subsequent reaction. ¹H NMR (400 MHz, CDCl₃) δ 7.92 (d, 1H), 7.82 (d, 1H), 7.54 (d, 2H), 7.46 (dd, 1H), 7.38 (dd, 1H), 7.19 (d, 2H), 6.43 (m, 1H), 6.28 (m, 1H), 5.79 (d, 1H), 3.60 (m, 4H), 2.72 (m, 4H), 2.65 (t, 2H), 2.49 (t, 2H), 1.90 (m, 2H). MS m/z 407 [C₂₃H₂₆N₄OS+1].

B. [(2-{4-[3-(4-Benzo[d]isothiazol-3-yl-piperazin-1-yl)-propyl]-phenylcarbamoyl}-ethyl)-methyl-amino]-acetic acid ethyl ester

A mixture of N-{4-[3-(4-Benzo[d]isothiazol-3-yl-piperazin-1-yl)-propyl]-phenyl}-acrylamide (2.80 g, 6.9 mmol), sarcosine ethyl ester hydrochloride (5.30 g, 34.5 mmol), triethylamine (3.48 g, 34.5 mmol), and 2,6-di-tert-butyl-p-cresol (100 mg) in 60 mL of methanol was refluxed at 90° C. overnight. After cooling, methanol was evaporated in vacuo. Ethyl acetate (400 mL) was added to the residue, washed with brine (3×50 mL), dried over sodium sulphate, and concentrated. Chromatography on silica gel column using dichloromethane:methanol (95:5) as eluent gave the desired product (3.18 g, 88.1%) as a yellowish oil, which was used immediately for the subsequent reaction. MS m/z524 [C₂₈H₃₇N₅O₃S+1].

C. 1-Methyl-4-oxo-pyrrolidine-3-carboxylic acid {4-[3-(4-benzo[d]isothiazol-3-yl-piperazin-1-yl)-propyl]-phenyl}-amide

Potassium t-butoxide (0.81 g, 7.2 mmol) was dissolved in 15 mL of dry tetrahydrofuran and cooled to 5-10° C. with ice-bath. To this mixture, [(2-{4-[3-(4-Benzo[d]isothiazol-3-yl-piperazin-1-yl)-propyl]-phenylcarbamoyl}-ethyl)-methyl-amino]-acetic acid ethyl ester (3.14 g, 6.0 mmol) in 15 mL of dry tetrahydrofuran was added slowly with stirring. The reaction mixture was stirred at the same temperature for 4 hours, then 20 mL of water was added, and the pH was adjusted to 7˜8 with 1N HCl. The product was extracted with ethyl acetate (3×400 mL), dried over sodium sulphate, and concentrated. The resulting solid was triturated with ether by sonication to give the desired product (2.0 g, 70.0%) as an off-white solid. ¹H NMR (400 MHz, CDCl₃) δ 8.64 (br s, 1H), 7.94 (d, 1H), 7.81 (d, 1H), 7.45 (m, 3H), 7.38 (dd, 1H), 7.19 (dd, 2H), 3.59 (m, 4H), 3.50 (m, 1H), 3.38-3.20 (m, 3H), 3.01 (m, 1H), 2.68 (m, 6H), 2.50 (s, 3H), 2.44 (dd, 2H), 1.85 (m, 2H). MS m/z478 [C₂₆H₃₁N₅O₂S+1].

D. 8-[3-(4-Benzo[d]isothiazol-3-yl-piperazin-1-yl)-propyl]-2-methyl-1,2,3,5-tetrahydro-pyrrolo[3,4-c]quinolin-4-one

A mixture of 1-methyl-4-oxo-pyrrolidine-3-carboxylic acid {4-[3-(4-benzo[d]isothiazol-3-yl-piperazin-1-yl)-propyl]-phenyl}-amide (0.81 g, 1.70 mmol) and polyphosphoric acid (PPA, 20 g) was heated at 130° C. with stirring under nitrogen for 3 h. After cooling, the mixture was poured into ice water, the resulting brownish gum was collected by filtration which was sonicated in 100 mL of saturated sodium bicarbonate solution for 1 hour, then extracted with dichloromethane (3×500 mL). The filtrate from the gum was basified with solid potassium hydroxide to pH 8˜8.5 with cooling bath, then extracted with dichloromethane (3×500 mL). All dichloromethane extracts were combined, dried over sodium sulphate, and concentrated. Chromatography on silica gel column using dichloromethane:methanol (4:1) as eluent gave the desired material (0.36 g, 46.2%) as an off-white solid. mp 206-208° C. ¹H NMR (400 MHz, CDCl₃) δ 10.60 (br s, 1H), 7.94 (d, 1H), 7.80 (d, 1H), 7.48 (dd, 1H), 7.38 (dd, 2H), 7.28 (m,1H), 7.21 (d, 1H), 4.28 (dd, 2H), 4.08 (dd, 2H), 3.60 (m, 4H), 2.79 (t, 2H), 2.68 (m, 7H), 2.46 (t, 2H), 1.95 (m, 2H). MS m/z 460 [C₂₆H₂₉N₅OS+1]. Anal. Calcd for C₂₆H₂₉N₅OS: C, 67.94; H, 6.36; N, 15.24. Found: C, 67.86; H, 6.00; N, 15.10.

### Example 173

### 8-[2-(4-BENZO[D]ISOTHIAZOL-3-YL-PIPERAZIN-1-YL)-ETHYL]-2-METHYL-1,2,3,5-TETRAHYDRO-PYRROLO[3,4-C]QUINOLIN-4-ONE

8-[2-(4-Benzo[d]isothiazol-3-yl-piperazin-1-yl)-ethyl]-2-methyl-1,2,3,5-tetrahydro-pyrrolo[3,4-c]quinolin-4-one was prepared in an analogous fashion (example 172) from 4-[2-(4-Benzo[d]isothiazol-3-yl-piperazin-1-yl)-ethyl]-phenylamine. mp 205-207° C. ¹H NMR (400 MHz, CDCl₃) δ 10.62 (br s, 1H), 7.94 (d, 1H), 7.81 (d, 1H), 7.48 (dd, 1H), 7.39 (m, 2H), 7.28 (m, 2H), 4.25 (m, 2H), 4.08 (m, 2H), 3.61 (m, 4H), 2.95 (m, 2H), 2.79 (m, 4H), 2.75 (m, 2H), 2.70 (s, 3H). MS m/z 446 [C₂₅H₂₇N₅OS+1]. Anal. Calcd for C₂₅H₂₇N₅OS.1.25 H₂O: C, 64.15; H, 6.35; N, 14.96. Found: C, 63.91; H, 5.85; N, 14.76.

### Example 174

### 8-[2-(4-BENZO[D]ISOTHIAZOL-3-YL-PIPERAZIN-1-YL)-ETHYL]-5-ETHYL-1,2,3.5-TETRAHYDRO-CYCLOPENTA[C]QUINOLIN-4-ONE

To a solution of 8-[2-(4-Benzo[d]isothiazol-3-yl-piperazin-1-yl)-ethyl]-1,2,3,5-tetrahydro-cyclopenta[c]quinolin-4-one (example 81, 0.10 g, 0.23 mmol) in DMF (1.5 mL) at room temperature was added NaH (10 mg, 0.24 mmol; as 60% suspension in oil). The resulting suspension was stirred at room temperature for 30 min and iodoethane (40.5 mg, 0.26 mmol) was introduced. The mixture was heated at 50° C. for 2 hours. It was allowed to cool to room temperature and poured into crushed ice. The precipitate was collected, washed with water, and purified by column chromatography (hexane:ethylacetate:methanol; 25:25:1) to obtain a solid (30 mg, 29%). ¹H NMR (400 MHz, CDCl₃): δ 7.95 (d, 1H), 7.85 (d, 1H), 7.55-7.30 (m, 5H), 4.40 (q, 2H), 3.70-3.55 (m, 4H), 3.20-3.10 (m, 2H), 3.00-2.90 (m, 4H), 2.85-2.70 (m, 6H), 2.25-2.15 (m, 2H), 1.35 (t, 3H).

### Example 175

### 8-[2-(4-BENZO[D]ISOTHIAZOL-3-YL-PIPERAZIN-1-YL)-ETHYL]-5-METHYL-1,2,3,5-TETRAHYDRO-CYCLOPENTA[C]QUINOLIN-4-ONE

An analogous procedure, as described for 8-[2-(4-Benzo[d]isothiazol-3-yl-piperazin-1-yl)-ethyl]-5-ethyl-1,2,3,5-tetrahydro-cyclopenta[c]quinolin-4-one (Example 174), was used to prepare compound the desired material (0.25 g, 60%) from 8-[2-(4-Benzo[d]isothiazol-3-yl-piperazin-1-yl)-ethyl]-1,2,3,5-tetrahydro-cyclopenta[c]quinolin-4-one (0.40 g, 0.96 mmol), NaH (40 mg, 1.02 mmol; as 60% oil suspension) and iodomethane (0.14 g, 1.02 mmol). ¹H NMR (400 MHz, CDCl₃) δ 7.95 (d, 1H), 7.80 (d, 1H), 7.55-7.30 (m, 5H), 3.75 (s, 3H), 3.65-3.55 (m, 4H), 3.20-3.15 (m, 2H), 3.00-2.90 (m, 4H), 2.80-2.70 (m, 6H), 2.25-2.15 (m, 2H).

### Example 176

### 8-[3-(4-BENZO[D]ISOTHIAZOL-3-YL-PIPERAZIN-1-YL)-PROPOXY]-1,2,3,5-TETRAHYDRO-CYCLOPENTA[C]QUINOLIN-4-ONE METHANESULFONIC ACID

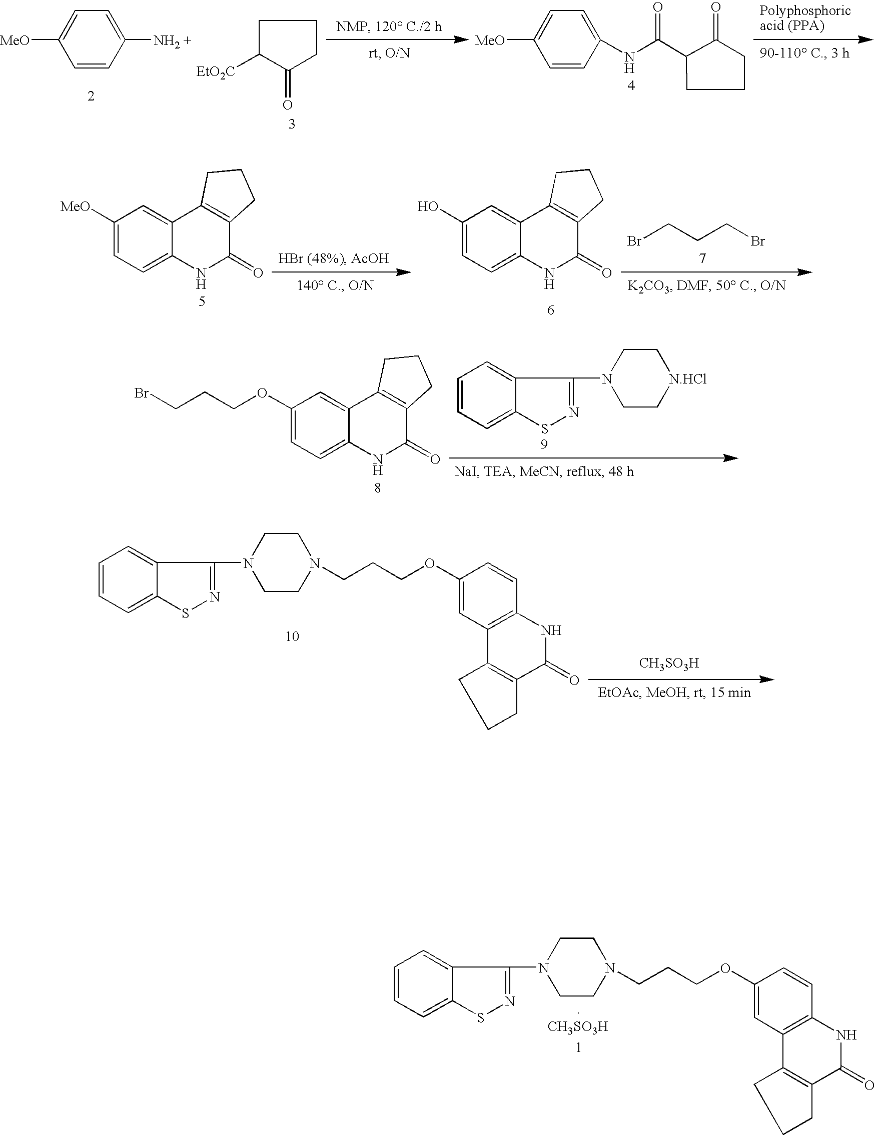

Referring to the scheme immediately above, a mixture of 2 (5.0 g, 40.6 mmol), and 3 (24 mL, 162 mmol) in NMP (50 mL) was heated to 120° C., and stirred for 2 h at the same temperature and stirring was continued overnight at room temperature. NMP was removed by vacuum distillation, and the residue was cooled to room temperature. The crude yellow solid was purified by crystallization from 95% ethanol to afford compound 4 (6.5 g, 69%). ¹H NMR (400 MHz, CDCl₃): δ 8.64 (s, 1H), 7.42 (d, 2H), 6.82 (d, 2H), 3.80 (s, 3H), 3.18 (t, 1H), 2.50-2.30 (m, 4H), 2.15-2.05 (m, 1H), 1.95-1.80 (m, 1H), MS m/z 233.85 [C₁₃H₁₅NO₃+H]⁺.

A mixture of 4 (2.0 g, 8.5 mmol) in polyphosphoric acid (20 g) was heated at 120° C. for 2.5 h with stirring. The cooled syrupy liquid was poured on to ice and polyphosphoric acid was neutralized with NaHCO₃. The solid was filtered from the aqueous layer and washed several times with water. The crude material was purified by crystallization from 95% ethanol to give 5 (1.0 g, 55%). ¹H NMR (400 MHz, DMSO-d₆): δ 11.50 (s, 1H), 7.25 (d, 1H), 7.10 (d, 1H), 7.00 (s, 1H), 3.80 (s, 3H), 3.08 (t, 2H), 2.75 (t, 2H), 2.18-2.05 (m, 2H), MS m/z 215.90 [C₁₃H₁₃NO₂+H]⁺.

A solution of 5 (600 mg, 2.8 mmol) in HBr (2.0 mL) and AcOH (3.0 mL) was heated to reflux at 140° C. overnight. After cooling the reaction mixture was poured into ice water and pH was adjusted to 4-5 with NaHCO₃. Organic material was extracted with EtOAc containing 5% MeOH (3×50 mL). The combined organics were washed with water and brine, and dried over MgSO₄. Filtration and evaporation of the solvent gave 6 as solid (300 mg, 53%). ¹H NMR (400 MHz, DMSO-d₆): δ 11.40 (s, 1H), 7.20 (d, 1H), 6.95 (d, 1H), 6.80 (s, 1H), 3.00 (t, 2H), 2.70 (t, 2H), 2.18-2.05 (m, 2H), MS m/z 202.04 [C₁₂H₁₁NO₂+H]⁺.

To a solution of 6 (3.30 g, 16.4 mmol) in anhydrous DMF was added K₂CO₃ (9.06 g, 65.6 mmol) followed by 1,3-dibromopropane 7 (8.3 mL, 82 mmol). The reaction mixture was heated at 50° C., and continued the stirring overnight. Cooled the reaction mixture to room temperature and quenched with water (100 mL). The organic compounds were extracted with CH₂Cl₂ containing 5% MeOH (3×100 mL). The combined organics were washed with 1N NaOH followed by water, and brine. The organic layer was dried over Na₂SO₄, filtered and concentrated in vacuo. The residue was then suspended in hexane and sonicated for 1 min, and filtered. The solid was rinsed with additional hexane and dried under vacuum to provide the compound 8 (900 mg, 17%). ¹H NMR (400 MHz, CDCl₃): δ 11.35 (br s, 1H), 7.32 (d, 1H), 7.18-7.10 (m, 1H), 6.95 (s, 1H), 4.18 (t, 2H), 3.62 (t, 2H), 3.18 (t, 2H), 3.00 (t, 2H), 2.28 (t, 2H), 2.20 (t, 2H). MS m/z 324.01 [C₁₅H₁₆BrN₄O₂+H]⁺.

A mixture of 8 (700 mg, 2.2 mmol), 9 (817 mg, 3.2 mmol), Nal (651 mg, 4.3 mmol), and triethylamine (1.5 mL, 10.9 mmol) in acetonitrile (50.0 mL), was heated to reflux for 48 h, and allowed to cool. The mixture was concentrated to dryness in vacuo. The residue was suspended in water (50 mL) and sonicated for 5 min, and then filtered through a sintered glass frit. The solid was rinsed with additional water, dried under vacuum, and purified by chromatography (silica gel, gradient 3-5% MeOH in CH₂Cl₂) to provide compound 10 (630 mg, 63%) as a white solid. ¹H NMR (400 MHz, CDCl₃): δ 10.75 (br s, 1H), 7.93 (d, 1H), 7.80 (d, 1H), 7.50-7.43 (m, 1H), 7.40-7.35 (m, 1H), 7.29-7.22 (m, 1H), 7.10 (d, 1H), 6.98 (s, 1H), 4.10 (t, 2H), 3.61-3.52 (m, 4H), 3.15 (t, 2H), 3.00 (t, 2H), 2.80-2.72 (m, 4H), 2.70 (t, 2H), 2.29-2.20 (m, 2H), 2.10-2.02 (m, 2H), MS m/z 460.97 [C₂₆H₂₈N₄O₂S+H]⁺.

Compound 10 (free base, 600 mg, 1.3 mmol) was dissolved in EtOAc (20.0 mL) and MeOH (2.0 mL), and then treated with MeSO₃H (84 μL, 1.3 mmol). The reaction mixture was stirred for 15 min at room temperature. The resulting solid was filtered and washed with EtOAc (20 mL) and Et₂O (20 mL) and dried in a vacuum oven at 70° C. to give brown solid 1 (610 mg, 78%). ¹H NMR (400 MHz, DMSO-d₆): δ 11.75 (s, 1H), 9.60 (br s, 1H), 8.20-8.10 (m, 2H), 7.60 (dd, 1H), 7.50 (dd, 1H), 7.30 (d, 1H), 7.15 (d, 1H), 7.00 (s, 1H), 4.20-4.11 (m, 4H), 3.78-3.72 (m, 2H), 3.45-3.30 (m, 6H), 3.10 (t, 2H), 2.80 (t, 2H), 2.35 (s, 3H), 2.25-2.02(4H). MS m/z 460.91 [C₂₆H₂₈N₄O₂S+H]⁺, Anal. Calcd. C₂₆H₂₈N₄O₂S.1.5 CH₃SO₃H: C 54.61, H 5.68, N 9.26. Found C 54.75, H 5.65, N 8.98.

1. A compound of the formula 1 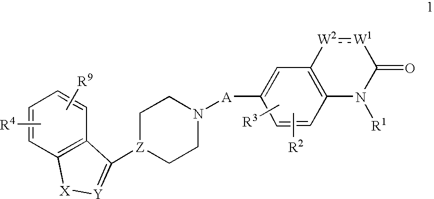
wherein X is sulfur, oxygen, SO, SO₂, CH₂ or NR¹⁰;
Y is nitrogen or CH;
Z is nitrogen or CH;
A is —(CH₂)ₘCH₂—, —(CH₂)ₘO—, —(CH₂)ₘNR¹¹—, or —(CH₂)ₘC(R¹²R¹³)—, wherein R¹² and R¹³ are independently selected from (C₁-C₄) alkyl optionally substituted with from one to three fluorine atoms, (C₁-C₄) alkoxy optionally substituted with from one to three fluorine atoms, hydroxy, and aminoalkyl;
or R¹² and R¹³, together with the carbon to which they are attached, form a carbonyl group;
m is an integer from one to four;
R⁴ and R⁹ are independently selected from hydrogen, (C₁-C₄) alkyl optionally substituted with from one to three fluorine atoms, (C₁-C₄) alkoxy optionally substituted with from one to three fluorine atoms, halogen, nitro, cyano, amino, (C₁-C₄) alkylamino and di-(C₁-C₄) alkylamino;
or, when X is NR¹⁰, one of R⁴ and R⁹ can form, together with the carbon to which it is attached, and together with R¹⁰ and the nitrogen to which it is attached, a heterocyclic ring containing from 4 to 7 ring members of which from 1 to 3 ring members are heteroatoms selected from nitrogen, oxygen and sulfur, and of which the remaining ring members are carbon, with the proviso that when R¹¹ forms a ring with one of R⁴ and R⁹, the other of R⁴ and R⁹ is absent;
R¹⁰ and R¹¹ are independently selected from hydrogen, (C₁-C₄) alkyl optionally substituted with from one to three fluorine atoms and (C₁-C₄) alkoxy optionally substituted with from one to three fluorine atoms;
R¹ is hydrogen, (C₁-C₄) alkyl optionally substituted with from one to three fluorine atoms, aryl, —C(O)R¹⁴ wherein R¹⁴ is aryl, (C₁-C₄) alkyl, aryl-(C₁-C₄)-alkyl-, or heteroaryl-(C₁-C₄)alkyl-, and wherein the alkyl moieties of the aryl-(C₁-C₄) alkyl- groups and the heteroaryl-(C₁-C₄) alkyl- groups can be optionally substituted with from one to three fluoro atoms, and wherein the aryl and heteroaryl moieties of these groups can optionally be substituted with one or more substituents, preferably with from zero to two substituents, independently selected from halo, nitro, amino, cyano, (C₁-C₆) alkyl optionally substituted with from one to three fluorine atoms and (C₁-C₆) alkoxy optionally substituted with from one to three fluorine atoms;
R² and R³ are independently selected from hydrogen, (C₁-C₄) alkyl, (C₁-C₄) alkoxy, halo, aryl, aryl-(C₁-C₄) alkyl-, heteroaryl and heteroaryl-(C₁-C₄) alkyl-, wherein the alkyl moieties of the (C₁-C₄) alkyl and (C₁-C₄) alkoxy groups can be optionally substituted with from one to three fluoro atoms and can also be independently optionally substituted with an amino or hydroxy substituent, and wherein alkyl moieties of the aryl-(C₁-C₄) alkyl- and heteroaryl-(C₁-C₄) alkyl groups can be optionally substituted with from one to three fluoro atoms, and wherein the aryl and heteroaryl moieties of these groups can optionally be substituted with one or more substituents, preferably from zero to two substituents, independently selected from halo, nitro, amino, cyano, (C₁-C₆) alkyl optionally substituted with from one to three fluorine atoms and (C₁-C₆) alkoxy optionally substituted with from one to three fluorine atoms;
or one of R² and R³ can form, together with the carbon to which it is attached, and together with the quinolinone ring carbon of W¹, a saturated or unsaturated heterocyclic ring containing from 4 to 7 ring members of which from 1 to 3 ring members can be heteroatoms selected from nitrogen, oxygen and sulfur, and of which the remaining ring members are carbon, with the proviso that when W¹ forms a ring with one of R² and R³, the other of R² and R³ is absent;
W¹ is CR⁵R⁶ and W² is CR⁷R⁸, and the broken line extending from W¹ to W² represents an optional double bond, with the proviso that when the there is a double bond between W¹ and W², R⁵ and R⁷ are absent;
R⁵, R⁶, R⁷, and R⁸ selected, independently, from hydrogen, halogen, nitro, cyano, amino, (C₁-C₄) alkylamino, di-(C₁-C₄) alkylamino, (C₁-C₄) alkyl optionally substituted with from one to three fluorine atoms, and (C₁-C₄) alkoxy optionally substituted with from one to three fluorine atoms;
or any two of R⁵, R⁶, R⁷, and R⁸ that are attached to carbon atoms, taken together with the carbon or carbons to which they are attached, form a (C₃-C₇) saturated or unsaturated carbocyclic ring, with the proviso that the quinolinone ring carbon of W¹ can not form a ring with two of R⁵, R⁶, R⁷, and R⁸ and also form a ring with R² or R³;
or a pharmaceutically acceptable salt of such compound.

2. A compound or salt according to claim 1, wherein A is —(CH₂)ₘO—.

3. A compound or salt according to claim 1, wherein A is —(CH₂)ₘNR¹¹.

4. A compound or salt according to claim 1, wherein X is CH₂.

5. A compound or salt according to claim 1, wherein X is NR.

6. A compound or salt according to claim 1, wherein X is sulfur.

7. A compound or salt according to claim 1, wherein X is SO or SO₂.

8. A compound or salt that is selected from the following compounds and their pharmaceutically acceptable salts:
6-[2-(4-benzo[d]isoxazol-3-yl-piperazin-1-yl)-ethyl]-4-methyl-3,4-dihydro-1H-quinolin-2-one;
6-[2-(4-benzo[d]isoxazol-3-yl-piperazin-1-yl)-ethyl]-4S-methyl-3,4-dihydro-1H-quinolin-2-one;
6-[2-(4-benzo[d]isoxazol-3-yl-piperazin-1-yl)-ethyl]-4R-methyl-3,4-dihydro-1H-quinolin-2-one;
6-[2-(4-benzo[d]isoxazol-3-yl-piperazin-1-yl)-ethyl]-1,4-dimethyl-3,4-dihydro-1H-quinolin-2-one;
6-[2-(4-benzo[d]isoxazol-3-yl-piperazin-1-yl)-ethyl]-4,4-dimethyl-3,4-dihydro-1H-quinolin-2-one;
6-[2-(4-benzo[d]isoxazol-3-yl-piperazin-1-yl)-ethyl]-1,4,4-trimethyl-3,4-dihydro-1H-quinolin-2-one;
6-[2-(4-benzo[d]isothiazol-3-yl-piperazin-1-yl)-ethyl]-4,4,8-trimethyl-3,4-dihydro-1H-quinolin-2-one;
6-[2-(4-benzo[d]isoxazol-3-yl-piperazin-1-yl)-ethyl]-3-methyl-3,4-dihydro-1H-quinolin-2-one;
6-[2-(4-benzo[d]isoxazol-3-yl-piperazin-1-yl)-ethyl]-3,3-dimethyl-3,4-dihydro-1H-quinolin-2-one;
6-[2-(4-benzo[d]isoxazol-3-yl-piperazin-1-yl)-ethyl]-3,4-dimethyl-3,4-dihydro-1H-quinolin-2-one;
6-[2-(4-benzo[d]isoxazol-3-yl-piperazin-1-yl)-ethyl]-1,3,3,4,4-pentamethyl-3,4-dihydro-1H-quinolin-2-one;
6-[2-(4-benzo[d]isoxazol-3-yl-piperazin-1-yl)-ethyl]-3,3,4-trimethyl-3,4-dihydro-1H-quinolin-2-one;
6-{2-[4-(1H-indazol-3-yl)-piperazin-1-yl]-ethyl}-4-methyl-3,4-dihydro-1H-quinolin-2-one;
6-{2-[4-(1H-indazol-3-yl)-piperazin-1-yl]-ethyl}-4,4-dimethyl-3,4-dihydro-1H-quinolin-2-one;
6-{2-[4-(1H-indazol-3-yl)-piperazin-1-yl]-ethyl}-3-methyl-3,4-dihydro-1H-quinolin-2-one;
6-{2-[4-(1H-indazol-3-yl)-piperazin-1-yl]-ethyl}-3,3-dimethyl-3,4-dihydro-1H-quinolin-2-one;
6-{2-[4-(1H-indazol-3-yl)-piperazin-1-yl]-ethyl}-3,4-dimethyl-3,4-dihydro-1H-quinolin-2-one;
6-[2-(4-benzo[d]isothiazol-3-yl-piperazin-1-yl)-ethyl]-1,3,3,4,4-pentamethyl-3,4-dihydro-1H-quinolin-2-one;
6-[2-(4-benzo[d]isothiazol-3-yl-piperazin-1-yl)-ethyl]-3,3,4-trimethyl-3,4-dihydro-1H-quinolin-2-one;
6-[2-(4-Benzo[d]isothiazol-3-yl-piperazin-1-yl)-ethyl]-4,4,8-trimethyl-3,4-dihydro-1H-quinolin-2-one, mesylate salt;
6-[2-(4-Benzo[d]isothiazol-3-yl-piperazin-1-yl)-ethyl]-7-chloro-4,4,8-trimethyl-3,4-dihydro-1H-quinolin-2-one methanesulfonate;
6-[2-(4-Benzo[d]isothiazol-3-yl-piperazin-1-yl)-ethyl]-7-fluoro-4,4,8-trimethyl-3,4-dihydro-1H-quinolin-2-one hydrochloride;
6-{3-[4-(1H-indazol-3-yl)-piperazin-1-yl]-propyl}-4,4-dimethyl-3,4-dihydro-1H-quinolin-2-one;
7-chloro-6-[3-(4-1,2-benzisothiazol-3-yl-piperazin-1-yl)-propyl]-4,4-dimethyl-3,4-dihydro-1H-quinolin-2-one;
7-chloro-6-[3-(4-1,2-benzisoxazol-3-yl-piperazin-1-yl)-propyl]-4,4-dimethyl-3,4-dihydro-1H-quinolin-2-one;
6-[3-(4-1,2-benzisothiazol-3-yl-piperazin-1-yl)-propyl]-4-methyl-3,4-dihydro-1H-quinolin-2-one;
6-[3-(4-1,2-benzisoxazol-3-yl-piperazin-1-yl)-propyl]-4-methyl-3,4-dihydro-1H-quinolin-2 one;
6-{3-[4-(1H-indazol-3-yl)-piperazin-1-yl]-propyl}-4-methyl-3,4-dihydro-1H-quinolin-2-one;
6-[3-(4-1,2-benzisothiazol-3-yl-piperazin-1-yl)-propyl]-3,3-dimethyl-3,4-dihydro-1H-quinolin-2-one;
6-[3-(4-1,2-benzisoxazol-3-yl-piperazin-1-yl)-propyl]-3,3-dimethyl-3,4-dihydro-1H-quinolin-2 one;
6-{3-[4-(1H-indazol-3-yl)-piperazin-1-yl]-propyl}-3,3-dimethyl-3,4-dihydro-1H-quinolin-2-one;
6-[3-(4-1,2-benzisothiazol-3-yl-piperazin-1-yl)-propyl]-3-methyl-3,4-dihydro-1H-quinolin-2-one;
6-[3-(4-1,2-benzisoxazol-3-yl-piperazin-1-yl)-propyl]-3-methyl-3,4-dihydro-1H-quinolin-2 one; and
6-{3-[4-(1H-indazol-3-yl)-piperazin-1-yl]-propyl}-3-methyl-3,4-dihydro-1H-quinolin-2-one.

9. A compound according to claim 1 wherein A is —(CH₂)ₘCH₂— wherein m is two.

10. A compound according to claim 1 wherein R⁴ is hydrogen and one or both of R² and R³ are hydrogen.

11. A compound according to claim 1 wherein R¹, R⁵, R⁶, R⁷ and R⁸ are selected, independently, from hydrogen and (C₁-C₃)alkyl.

12. A compound according to claim 1, wherein one or both of R² and R³ are hydrogen.

13. A pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising an amount of a compound according to claim 1, or a pharmaceutically acceptable salt thereof, that is effective in treating such disorder or condition, and a pharmaceutically acceptable carrier.

14. A method for treating a disorder or condition selected from single episodic or recurrent major depressive disorders, dysthymic disorders, depressive neurosis and neurotic depression, melancholic depression syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising administering to a mammal in need of such treatment an amount of a compound according to claim 1, or a pharmaceutically acceptable salt thereof, that is effective in treating such disorder or condition.

15. A method according to claim 14, wherein the disorder or condition that is being treated is selected from major depression, single episode depression, recurrent depression, child abuse induced depression, postpartum depression, dysthymia, cyclothymia and bipolar disorder.

16. A method according to claim 14, wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

17. A method according to claim 14, wherein the disorder or condition that is being treated is selected from autism, pervasive development disorder, and attention deficit hyperactivity disorder.

18. A method according to claim 14, wherein the disorder or condition that is being treated is selected from generalized anxiety disorder, panic disorder, obsessive-compulsive disorder, post-traumatic stress disorder, and phobias, including social phobia, agoraphobia, and specific phobias.

19. A method according to claim 14, wherein the compound according to claim 1 is administered to a human for the treatment of any two or more including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant comorbid disorders or conditions selected from those disorders and conditions referred to in any of the above methods.

20. A method according to claim 14, wherein the disorder or condition being treated is schizophrenia with concomitant depression.

21. A method according to claim 14, wherein the disorder or condition being treated is schizophrenia with concomitant anxiety.

22. A method of treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising administering to said mammal:
(a) a compound according to claim 1 or a pharmaceutically acceptable salt thereof; and
(b) another pharmaceutically active compound that is an antidepressant or an anti-anxiety agent, or a pharmaceutically acceptable salt thereof;
wherein the active agents “a” and “b” are present in amounts that render the combination effective in treating such disorder or condition.

23. A pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal comprising:
(a) a compound according to claim 1 or a pharmaceutically acceptable salt thereof;
(b) another pharmaceutically active agent that is an antidepressant or an anti-anxiety agent; and
(c) a pharmaceutically acceptable carrier.

24. A method according to claim 22, wherein the disorder or condition being treated is schizophrenia.

25. A method according to claim 22, wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

26. A method according to claim 22, wherein the disorder or condition that is being treated is selected from autism, pervasive development disorder, and attention deficit hyperactivity disorder.

27. A method according to claim 22, wherein the disorder or condition that is being treated is selected from generalized anxiety disorder, panic disorder, obsessive-compulsive disorder, post-traumatic stress disorder, and phobias, including social phobia, agoraphobia, and specific phobias.

28. A method according to claim 22, wherein the active agents “a” and “b” are administered to a human for the treatment of any two or more comorbid disorders or conditions selected from those disorders and conditions referred to in any of the above methods.

29. A method according to claim 22, wherein the disorder or condition being treated is schizophrenia with concomitant depression.

30. A method according to claim 22, wherein the disorder or condition being treated is schizophrenia with concomitant anxiety.

31. A pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising an amount of a compound according to claim 8, or a pharmaceutically acceptable salt thereof, that is effective in treating such disorder or condition, and a pharmaceutically acceptable carrier.

32. A method for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scoft syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising administering to a mammal in need of such treatment an amount of a compound according to claim 8, or a pharmaceutically acceptable salt thereof, that is effective in treating such disorder or condition.

33. A method according to claim 32, wherein the disorder or condition that is being treated is selected from major depression, single episode depression, recurrent depression, child abuse induced depression, postpartum depression, dysthymia, cyclothymia and bipolar disorder.

34. A method according to claim 32, wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

35. A method according to claim 32, wherein the disorder or condition that is being treated is selected from autism, pervasive development disorder, and attention deficit hyperactivity disorder.

36. A method according to claim 32, wherein the disorder or condition that is being treated is selected from generalized anxiety disorder, panic disorder, obsessive-compulsive disorder, post-traumatic stress disorder, and phobias, including social phobia, agoraphobia, and specific phobias.

37. A method according to claim 32, wherein the compound according to claim 8 is administered to a human for the treatment of any two or more comorbid disorders or conditions selected from those disorders and conditions referred to in any of the above methods.

38. A method according to claim 32, wherein the disorder or condition being treated is schizophrenia with concomitant depression.

39. A method according to claim 32, wherein the disorder or condition being treated is schizophrenia with concomitant anxiety.

40. A method of treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising administering to said mammal:
(a) a compound according to claim 8 or a pharmaceutically acceptable salt thereof; and
(b) another pharmaceutically active compound that is an antidepressant or an anti-anxiety agent, or a pharmaceutically acceptable salt thereof;
wherein the active agents “a” and “b” are present in amounts that render the combination effective in treating such disorder or condition.

41. A pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal comprising:
(a) a compound according to claim 8 or a pharmaceutically acceptable salt thereof;
(b) another pharmaceutically active agent that is an antidepressant or an anti-anxiety agent; and
(c) a pharmaceutically acceptable carrier.

42. A method according to claim 40, wherein the disorder or condition being treated is schizophrenia.

43. A method according to claim 40, wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

44. A method according to claim 40, wherein the disorder or condition that is being treated is selected from autism, pervasive development disorder, and attention deficit hyperactivity disorder.

45. A method according to claim 40, wherein the disorder or condition that is being treated is selected from generalized anxiety disorder, panic disorder, obsessive-compulsive disorder, post-traumatic stress disorder, and phobias, including social phobia, agoraphobia, and specific phobias.

46. A method according to claim 40, wherein the active agents “a” and “b” are administered to a human for the treatment of any two or more comorbid disorders or conditions selected from those disorders and conditions referred to in any of the above methods.

47. A method according to claim 40, wherein the disorder or condition being treated is schizophrenia with concomitant depression.

48. A method according to claim 40, wherein the disorder or condition being treated is schizophrenia with concomitant anxiety.
